(19) United States
(12) Patent Application Publication
(10) Pub. No.: US 2006/0276515 A1
(43) Pub. Date: Dec. 7, 2006

(54) Soluble Epoxide Hydrolase Inhibitors and Methods of Using Same

(76) Inventors: Charles Lawrence Cywin, Bethel, CT (US); Stephane De Lombaert, Madison, CT (US); Anne Bettina Eldrup, Danbury, CT (US); Richard Harold Ingraham, New Fairfield, CT (US); Fariba Soleymanzadeh, Danbury, CT (US); Steven John Taylor, Southbury, CT (US); Mario G. Cardozo, San Francisco, CA (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(21) Appl. No.: 11/381,015

(22) Filed: May 1, 2006

Related U.S. Application Data
(60) Provisional application No. 60/678,828, filed on May 6, 2005.

(51) Int. Cl. A61K 31/4439; A61K 31/4415; A61K 31/277; A61K 31/165

(52) U.S. Cl. 514/343; 514/350; 514/617; 514/521; 546/276.4; 558/410; 564/152; 546/291

(57) ABSTRACT

Disclosed are compounds active against soluble epoxide hydrolase (sEH), compositions thereof and methods of using and making same.

### APPLICATION DATA

This applicationm claims benefit to U.S. provisional application Ser. No. 60/678,828 filed May 6, 2005.

### BACKGROUND OF THE INVENTION

1. Technical Field

This invention relates to compounds possessing anti-sEH activity and methods of using soluble epoxide hydrolase (sEH) inhibitors for diseases related to cardiovascular disease.

2. Background Information

Epoxide hydrolases are a group of enzymes ubiquitous in nature, detected in species ranging from plants to mammals. These enzymes are functionally related in that they all catalyze the addition of water to an epoxide, resulting in a diol. Epoxide hydrolases are important metabolizing enzymes in living systems and their diol products are frequently found as intermediates in the metabolic pathway of xenobiotics. Epoxide hydrolases are therefore important enzymes for the detoxification of epoxides by conversion to their corresponding, non-reactive diols.

In mammals, several types of epoxide hydrolases have been characterized including soluble epoxide hydrolase (sEH), also referred to as cytosolic epoxide hydrolase, cholesterol epoxide hydrolase, LTA₄ hydrolase, hepoxilin hydrolase, and microsomal epoxide hydrolase (Fretland and Omiecinski, Chemico-Biological Interactions, 129: 41-59 (2000)). Epoxide hydrolases have been found in all tissues examined in vertebrates including heart, kidney and liver (Vogel, et al., Eur J. Biochemistry, 126: 425-431 (1982); Schladt et al., Biochem. Pharmacol., 35: 3309-3316 (1986)). Epoxide hydrolases have also been detected in human blood components including lymphocytes (e.g. T-lymphocytes), monocytes, erythrocytes, platelets and plasma. In the blood, most of the sEH detected was present in lymphocytes (Seidegard et al., Cancer Research, 44: 3654-3660 (1984)).

The epoxide hydrolases differ in their specificity towards epoxide substrates. For example, sEH is selective for aliphatic epoxides such as epoxide fatty acids while microsomal epoxide hydrolase (mEH) is more selective for cyclic and arene epoxides. The primary known physiological substrates of sEH are four regioisomeric cis epoxides of arachidonic acid, 5,6-, 8,9-, 11,12-, and 14,15-epoxyeicosatrienoic acid, also known as epoxyeicosatrienoic acids or EETs. Also known to be substrates for sEH are epoxides of linoleic acid known as leukotoxin or isoleukotoxin. Both the EETs and the leukotoxins are generated by members of the cytochrome P450 monooxygenase family (Capdevila, et al., J. Lipid Res., 41: 163-181 (2000)).

EETs function as chemical autocrine and paracrine mediators in the cardiovascular and renal systems (Spector, et al, Progress in Lipid Research, 43: 55-90 (2004); Newman, et al., Progress in Lipid Research 44: 1-51 (2005)). EETs appear to be able to function as endothelial derived hyperpolarizing factor (EDHF) in various vascular beds due to their ability to cause hyperpolarization of the membranes of vascular smooth muscle cells with resultant vasodilation (Weintraub, et al., Circ. Res., 81: 258-267 (1997)). EDHF is synthesized from arachidonic acid by various cytochrome P450 enzymes in endothelial cells proximal to vascular smooth muscle (Quilley, et al., Brit. Pharm., 54: 1059 (1997); Quilley and McGiff, TIPS, 21: 121-124 (2000)); Fleming and Busse, Nephrol. Dial. Transplant, 13: 2721-2723 (1998)). In the vascular smooth muscle cells EETs provoke signaling pathways which lead to activation of BK_{Ca}²⁺ channels (big Ca²⁺ activated potassium channels) and inhibition of L-type Ca²⁺ channels, ultimately resulting in hyperpolarization of membrane potential, inhibition of Ca²⁺ influx and relaxation (Li et al., Circ. Res., 85: 349-356 (1999)). Endothelium dependent vasodilation has been shown to be impaired in different forms of experimental hypertension as well as in human hypertension (Lind, et al., Blood Pressure, 9: 4-15 (2000)). Impaired endothelium dependent vasorelaxation is also a characteristic feature of the syndrome known as endothelial dysfunction (Goligorsky, et. al., Hypertension, 37[part 2]:744-748 (2001)). Endothelial dysfunction plays a significant role in a large number of pathological conditions including type 1 and type 2 diabetes, insulin resistance syndrome, hypertension, atherosclerosis, coronary artery disease, angina, ischemia, ischemic stroke, Raynaud's disease and renal disease. Hence, it is likely that enhancement of EETs concentration would have a beneficial therapeutic effect in patients where endothelial dysfunction plays a causative role. Other effects of EETs that may influence hypertension involve effects on kidney function. Levels of various EETs and their hydrolysis products, the DHETs, increase significantly both in the kidneys of spontaneously hypertensive rats (SHR) (Yu, et al., Circ. Res. 87: 992-998 (2000)) and in women suffering from pregnancy induced hypertension (Catella, et al., Proc. Natl. Acad. Sci. U.S.A., 87: 5893-5897 (1990)). In angiotensin II infused rats the treatment with a selective sEH inhibitor attenuated the afferent arteriolar diameter in the kidney and lowered urinary albumin secretion, a marker of compromised renal function, suggesting antihypertensive and renal vascular protective effects of increased EETs levels (Zhao, et al, 15: 1244-1253 (2004)). In the spontaneously hypertensive rat model, both cytochrome P450 and sEH activities were found to increase (Yu et al., Molecular Pharmacology, 57: 1011-1020 (2000)). Addition of a known sEH inhibitor was shown to decrease the blood pressure to normal levels. Furthermore, administration of a selective sEH inhibitor to angiotensin II treated rats was demonstrated to lower systolic blood pressure (Imig, et al, Hypertension, 39: 690-694 (2002)). Finally, male soluble epoxide hydrolase null mice exhibited a phenotype characterized by lower blood pressure than their wild-type counterparts (Sinal, et al., J. Biol. Chem., 275: 40504-40510 (2000)).

EETs, especially 11,12-EET, also have been shown to exhibit anti-inflammatory properties (Node, et al., Science, 285: 1276-1279 (1999); Campbell, TIPS, 21: 125-127 (2000); Zeldin and Liao, TIPS, 21: 127-128 (2000)). Node, et al. have demonstrated 11,12-EET decreases expression of cytokine induced endothelial cell adhesion molecules, especially VCAM-1. They further showed that EETs prevent leukocyte adhesion to the vascular wall and that the mechanism responsible involves inhibition of NF-κB and IκB kinase. Vascular inflammation plays a role in endothelial dysfunction (Kessler, et al., Circulation, 99: 1878-1884 (1999)). Hence, the ability of EETs to inhibit the NF-κB pathway should also help ameliorate this condition. In addition, the administration of EETs and/or the administration of a selective sEH inhibitor was demonstrated to attenuate tobacco smoke induced inflammation, as assessed total bronchoalveolar lavage cell numbers and concomittant reduction in neutrophils, alveolar macrophages, and lymphocytes (Smith, et al, 102: 2186-2191 (2005)).

In addition to the physiological effect of some substrates of sEH (EETs, mentioned above), some diols, i.e. DHETs, produced by sEH may have potent biological effects. For example, sEH metabolism of epoxides produced from linoleic acid (leukotoxin and isoleukotoxin) produces leukotoxin and isoleukotoxin diols (Greene, et al., Arch. Biochem. Biophys. 376(2): 420-432 (2000)). These diols were shown to be toxic to cultured rat alveolar epithelial cells, increasing intracellular calcium levels, increasing intercellular junction permeability and promoting loss of epithelial integrity (Moghaddam et al., Nature Medicine, 3: 562-566 (1997)). Therefore these diols could contribute to the etiology of diseases such as adult respiratory distress syndrome where lung leukotoxin levels have been shown to be elevated (Ishizaki, et al., Pulm. Pharm. & Therap., 12: 145-155 (1999)). Hammock, et al. have disclosed the treatment of inflammatory diseases, in particular adult respiratory distress syndrome and other acute inflammatory conditions mediated by lipid metabolites, by the administration of inhibitors of epoxide hydrolase (WO 98/06261; U.S. Pat. No. 5,955,496).

A number of classes of sEH inhibitors have been identified. Among these are chalcone oxide derivatives (Miyamoto, et al. Arch. Biochem. Biophys., 254: 203-213 (1987)) and various trans-3-phenylglycidols (Dietze, et al., Biochem. Pharm. 42: 1163-1175 (1991); Dietze, et al., Comp. Biochem. Physiol. B, 104: 309-314 (1993)).

More recently, Hammock et al. have disclosed certain biologically stable inhibitors of sEH for the treatment of inflammatory diseases, for use in affinity separations of epoxide hydrolases and in agricultural applications (U.S. Pat. No. 6,150,415). The Hammock '415 patent also generally describes that the disclosed pharmacophores can be used to deliver a reactive functionality to the catalytic site, e.g., alkylating agents or Michael acceptors, and that these reactive functionalities can be used to deliver fluorescent or affinity labels to the enzyme active site for enzyme detection (col. 4, line 66 to col. 5, line 5). Certain urea and carbamate inhibitors of sEH have also been described in the literature (Morisseau et al., Proc. Natl. Acad. Sci., 96: 8849-8854 (1999); Argiriadi et al., J. Biol. Chem., 275 (20): 15265-15270 (2000); Nakagawa et al. Bioorg. Med. Chem., 8: 2663-2673 (2000); US 2005/0026844 and Kim, et al., J. Med. Chem. 47(8): 2110-2122 (2004) both of which describe inhibitors with additional, tethered oxo pharmacophores).

WO 00/23060 discloses a method of treating immunological disorders mediated by T-lymphocytes by administration of an inhibitor of sEH. Several 1-(4-aminophenyl)pyrazoles are given as examples of inhibitors of sEH.

U.S. Pat. No. 6,150,415 to Hammock is directed to a method of inhibiting an epoxide hydrolase, using compounds having the structure 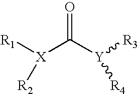
wherein X and Y is each independently nitrogen, oxygen, or sulfur, and X can further be carbon, at least one of R1-R4 is hydrogen, R2 is hydrogen when X is nitrogen but is not present when X is sulfur or oxygen, R4 is hydrogen when Y is nitrogen but is not present when Y is sulfur or oxygen, R1 and R3 is each independently H, C1-20 substituted or unsubstituted alkyl, cycloalkyl, aryl, acyl, or heterocyclic. Related to the Hammock patent is U.S. Pat. No. 6,531,506 to Kroetz et al. which claims a method of treating hypertension using of an inhibitor of epoxide hydrolase, also claimed are methods of treating hypertension using compounds similar to those described in the Hammock patent. Neither of these patents teaches or suggests methods of treating cardiovascular diseases using the particular sEH inhibitors described herein.

As outlined in the discussion above, inhibitors of sEH are useful therefore, in the treatment of cardiovascular diseases such as endothelial dysfunction either by preventing the degradation of sEH substrates that have beneficial effects or by preventing the formation of metabolites that have adverse effects.

All references cited above and throughout this application are incorporated herein by reference in their entirety.

### BRIEF SUMMARY OF THE INVENTION

It is therefore an object of the invention to provide compounds active as sEH inhibitors of the formulas III and IV as described herein below.

It is a further object of the invention to provide a method of treating hypertension by administering to a patient a compound of the formulas I, II, III or IV as described herein below.

It is yet a further object to provide methods of making the compounds described herein below.

### DETAILED DESCRIPTION OF THE INVENTION

In one generic aspect of the invention, there is provided a method of treating hypertension comprising administering to a patient an effective amount of a compound of the formula (I): 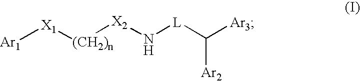
wherein:
n is 0 or 1;
X₁ is bond or a heteroatom chosen from O, S or a bond;
X₂ is —C(O)—;
L is an ethylene linking group optionally substituted by hydoxy, amino, lower alkoxy, lower alkylamino, lower alkylthio or 1-3 fluorine atoms;
Ar₁ is carbocycle, heteroaryl or heterocyclyl optionally substituted by Y;
Ar₂ and Ar₃ are carbocycle, heteroaryl or heterocyclyl each optionally substituted by one or more halogen, lower alkylS(O)ₘ, NR₂R₃—C(O)—, lower alkoxy or carboxamide;
R₁ is hydrogen or lower alkyl;
wherein the group —(CH₂)ₙ— in the formula (I) is optionally substituted by lower alkyl;
Y is chosen from
lower alkyl, lower alkoxy, lower alkenyl, lower acyl, lower alkyl(OH), —NR₂R₃;
or Y is a cyclic group chosen from heterocycle, heteroaryl and carbocycle;
each Y where possible is optionally substituted by one to three oxo, lower acyl, halogen, nitrile, lower alkylS(O)ₘ—, lower alkoxycarbonyl, NR₂R₃—C(O)—, —NR₂R₃, lower alkyl, C₃₋₆ cycloalkylC₀₋₂alkyl, hydroxy, lower alkoxy, aryloxy, arylC₀₋₄ alkyl, heteroaryl C₀₋₄ alkyl and heterocycle C₀₋₄alkyl, each above-listed heterocycle, heteroaryl and aryl group is optionally substituted by one to three hydroxy, oxo, lower alkyl, lower alkoxy, lower alkoxycarbonyl, NR₂R₃—C(O)— or lower acyl;
each R₂ and R₃ are independently hydrogen, arylC₀₋₄ alkyl, heteroaryl C₀₋₄ alkyl, heterocycle C₀₋₄alkyl, C₁₋₂ acyl, aroyl and lower alkyl optionally substituted by lower alkylS(O)ₘ—, lower alkoxy, hydroxy or mono or diC₁₋₃ alkyl amino;
or R₂ and R₃ optionally combine with the nitrogen atom to which they are attached to form a heterocyclic ring;
m is 0, 1 or 2;
or the pharmaceutically acceptable salts thereof.

In another embodiment of the invention there is provided a method of treating hypertension with compounds of the formula (I) as described immediately above, and wherein:

Ar₁ is cyclohexyl, phenyl; ademantyl, norbonyl,

or

heteroaryl chosen from pyridinyl, pyridinyl N-oxide, isoquinolinyl, quinolinyl, pyridazinyl and pyrimidinyl,

or

heterocyclyl chosen from piperidinyl, tetrahydropyranyl, morpholinyl, pyrrolidinyl, tetrahydrofuranyl, pyrrolidinonyl and benztriazolyl;

each Ar₁ is optionally substituted by Y;

Ar₂ and Ar₃ are each phenyl or pyridinyl optionally substituted by one or more lower alkoxy, F, Cl, lower alkylS(O)₂, lower alkyl-NH—C(O)— or carboxamide;

L is an ethylene linking group.

In another embodiment of the invention there is provided a method of treating hypertension with compounds of the formula (I) as described immediately above, and wherein:

Ar₂ and Ar₃ are each phenyl or pyridinyl substituted by one or more lower alkoxy, F, Cl, CH₃—S(O)₂—, CH₃—NH—C(O)— or carboxamide.

In another generic aspect of the invention, there is provided a method of treating hypertension comprising administering to a patient an effective amount of a compound of the formula (II): 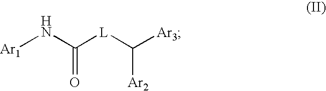
wherein:
Ar₁ is carbocycle, heteroaryl or heterocyclyl optionally substituted by Y;
Ar₂ and Ar₃ are each carbocycle optionally substituted by halogen, lower alkoxy, lower alkylS(O)ₘ, NR₂R₃—C(O)— or carboxamide;
L is an ethylene linking group optionally substituted by hydoxy, amino, lower alkoxy, lower alkylamino, lower alkylthio or 1-3 fluorine atoms;
Y is chosen from
lower alkyl, lower alkoxy, lower alkenyl, lower acyl, lower alkyl(OH), —NR₂R₃;
or Y is a cyclic group chosen from heterocycle, heteroaryl and carbocycle;
each Y where possible is optionally substituted by one to three oxo, lower acyl, halogen, nitrile, lower alkylS(O)ₘ—, lower alkoxycarbonyl, NR₂R₃—C(O)—, —NR₂R₃, lower alkyl, C₃₋₆ cycloalkylC₀₋₂alkyl, hydroxy, lower alkoxy, aryloxy, arylC₀₋₄ alkyl, heteroaryl C₀₋₄ alkyl and heterocycle C₀₋₄alkyl, each above-listed heterocycle, heteroaryl and aryl group is optionally substituted by one to three hydroxy, oxo, lower alkyl, lower alkoxy, lower alkoxycarbonyl, NR₂R₃—C(O)— or lower acyl;
each R₂ and R₃ are independently hydrogen, arylC₀₋₄ alkyl, heteroaryl C₀₋₄ alkyl, heterocycle C₀₋₄alkyl, C₁₋₂ acyl, aroyl and lower alkyl optionally substituted by lower alkylS(O)ₘ—, lower alkoxy, hydroxy or mono or diC₁₋₃ alkyl amino;
or R₂ and R₃ optionally combine with the nitrogen atom to which they are attached to form a heterocyclic ring;
m is 0, 1 or 2;
or the pharmaceutically acceptable salts thereof.

In another embodiment of the invention there is provided a method of treating hypertension with compounds of the formula (II) as described immediately above, and wherein:

Ar₁ is cyclohexyl, phenyl, adamantyl, norbornyl,

or

heteroaryl chosen from pyridinyl, pyridinyl N-oxide, isoquinolinyl, quinolinyl, pyridazinyl and pyrimidinyl,

or

heterocyclyl chosen from piperidinyl, tetrahydropyranyl, morpholinyl, pyrrolidinyl, tetrahydrofuranyl, pyrrolidinonyl and benztriazolyl;

each optionally substituted by Y;

Ar₂ and Ar₃ are each phenyl or pyridinyl optionally substituted by one or more lower alkoxy, F, Cl, lower alkylS(O)₂, lower alkyl-NH—C(O)— or carboxamide;

L is an ethylene linking group.

In another embodiment of the invention there is provided a method of treating hypertension with compounds of the formula (II) as described immediately above, and wherein:

Ar₂ and Ar₃ are each phenyl or pyridinyl substituted by one or more lower alkoxy, F, Cl, CH₃—S(O)₂—, CH₃—NH—C(O)— or carboxamide.

In another generic aspect of the invention, there is provided a compound of the formula (III):

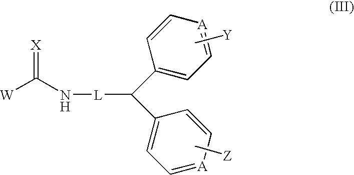

Each A is independently nitrogen or C—H such that each of the ring of which A is a member may be pyridinyl or phenyl, said pyridinyl or phenyl are optionally substituted by Y or Z;

Y and Z on their respective rings are in the meta or para position, and are independently F, Cl, Br, CN, OR, R, —S(O)₂R, —C(O)NRR or —S(O)₂NRR, wherein R is independently hydrogen or lower alkyl unsubstituted or substituted with hydroxy, amino, C₁₋₄ alkoxy, C₁₋₄ alkylamino, C₁₋₄ alkylthio, or one to three fluorine atoms;

L is an ethylene linker optionally substituted with hydroxy, amino, C₁₋₄ alkoxy C₁₋₄ alkylamino, C₁₋₄ alkylthio, or one to three fluorine atoms;

X is O or S;

W is chosen from phenyl, 2-pyridinyl, 3-pyridinyl, 4-pyridinyl, pyrazinyl, 3-pyridazinyl, 4-pyridazinyl, naphthyl, quinolinyl and isoquinolinyl each optionally with one to three substituents independently chosen from: halogen, hydroxy, amino, cyano, carboxy, carboxamido, C₁₋₄ alkyl unsubstitued or substituted with one to three halogen atoms, C₃₋₆ cycloalkyl unsubstitued or substituted with one to three halogen atoms, C₂₋₄ alkynyl, C₁₋₄ alkyloxycarbonyl, C₁₋₄ alkylamidocarbonyl, C₁₋₄ dialkylamidocarbonyl, C₁₋₄ alkylamino, C₁₋₄ dialkylamino, C₃₋₆ cycloalkylamino, di(C₃₋₆ cycloalkyl)amino, C₁₋₄ alkylsulfonyl, C₁₋₄ alkylheterocylyl, phenyl, or heterocylyl;

with the proviso that if the phenyl or pyridinyl rings possessing the aforementioned A are either unsubstituted or both substituted by halogen, then W must be substituted by any of the above-listed substituents for W;

or the pharmaceutically acceptable salts thereof.

In another generic aspect of the invention, there is provided a compound of the formula (IV): 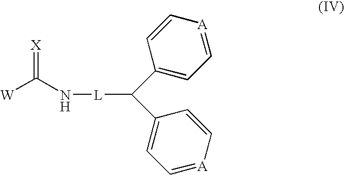
wherein for the Formula (IV), the component 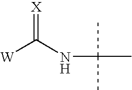
is chosen from A1-A8 in the table I below; in combination with any component

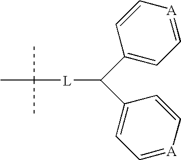

chosen from B1-B10 in the table I below;

| | 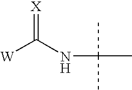 |
|---|---|
| A1 | 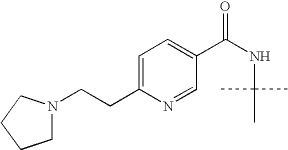 |
| A2 | 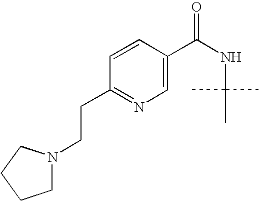 |
| A3 | 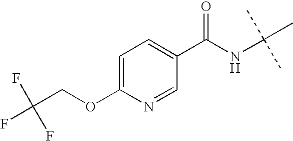 |
| A4 | 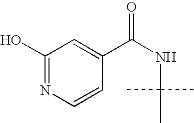 |
| A5 | 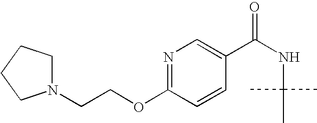 |
| A6 | 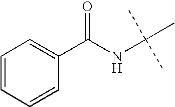 |
| A7 | 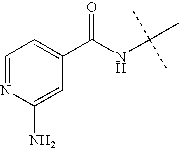 |
| A8 | 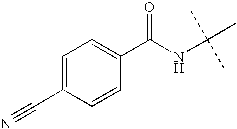 |
| A9 | |
| A10 | |
| | 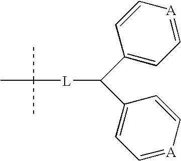 |
| B1 | 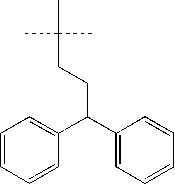 |
| B2 | 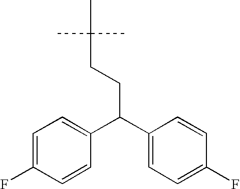 |
| B3 | 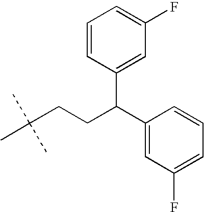 |
| B4 | 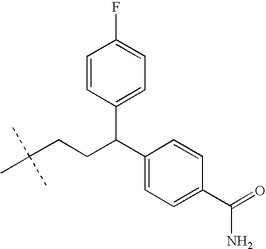 |
| B5 | 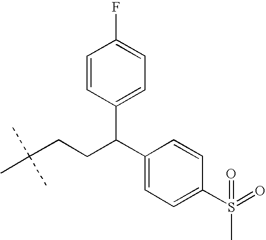 |
| B6 | 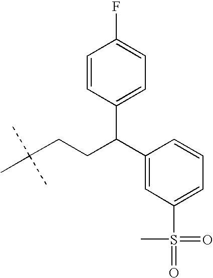 |
| B7 | 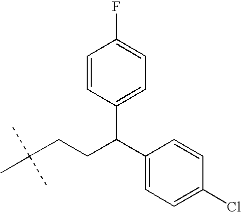 |
| B8 | 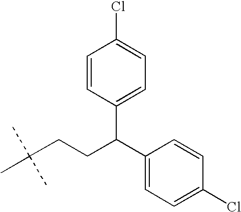 |
| B9 | 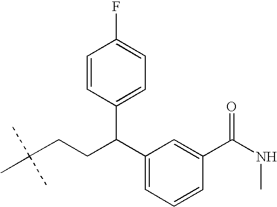 |
| B10 | 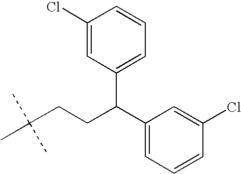 |

or the pharmaceutically acceptable salts thereof,
with the proviso that if 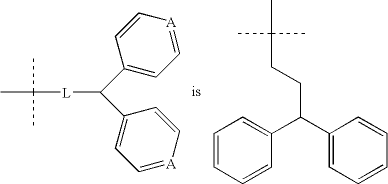
then 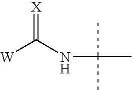
cannot be

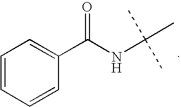

In another embodiment of the invention there is provided the following compounds which can be made according to the general synthetic procedures and examples which follow:

TABLE II

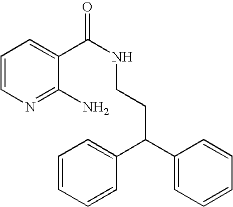

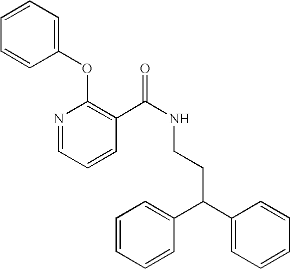

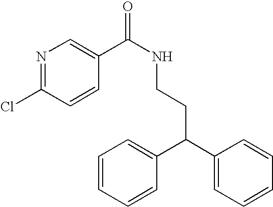

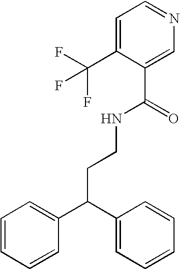

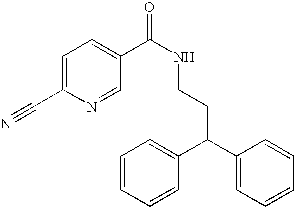

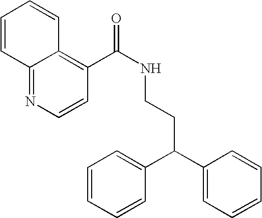

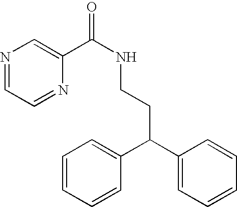

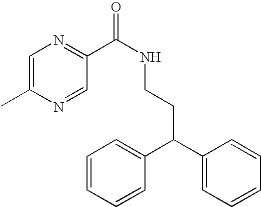

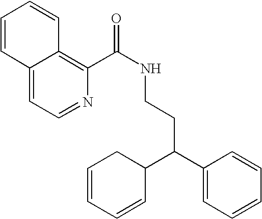

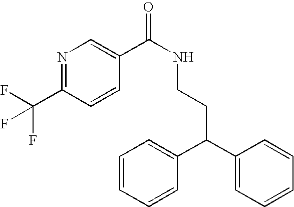

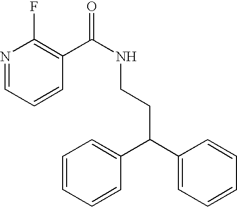

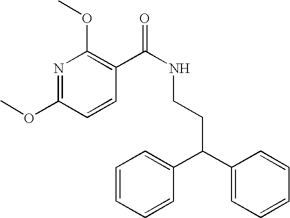

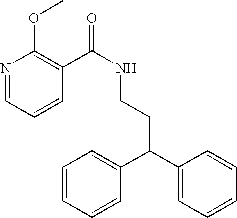

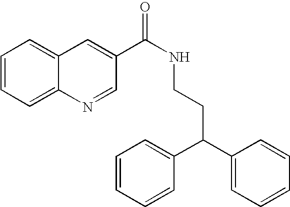

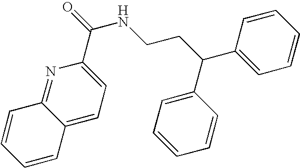

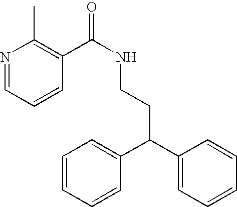

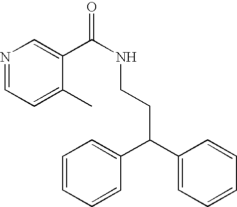

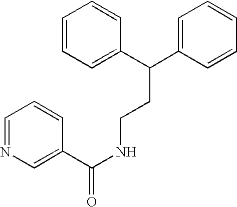

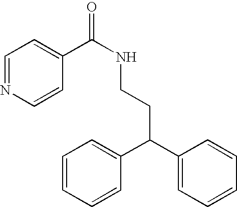

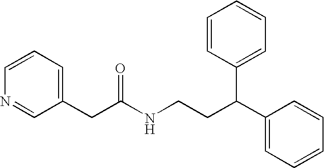

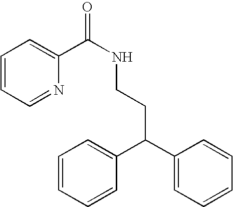

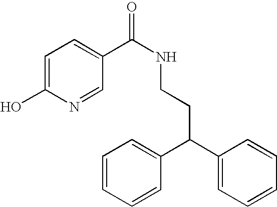

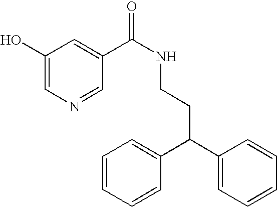

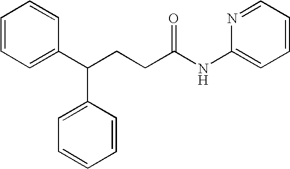

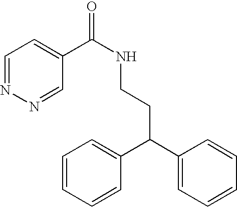

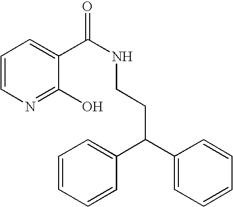

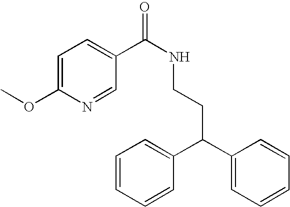

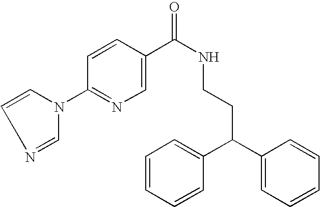

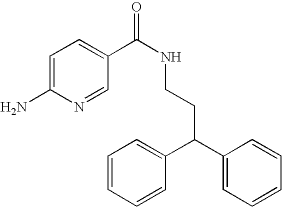

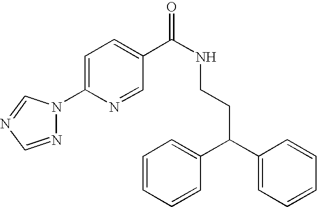

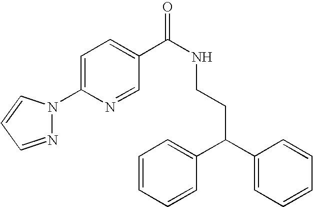

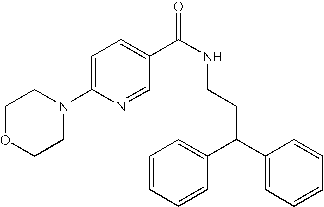

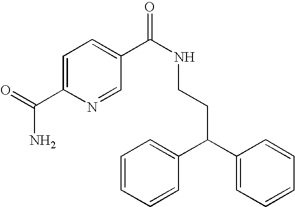

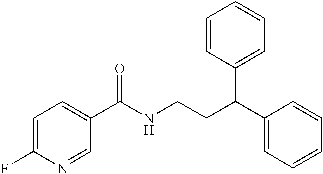

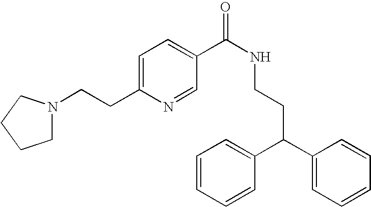

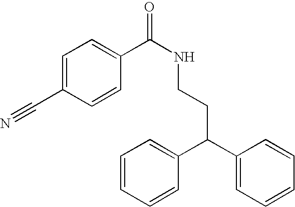

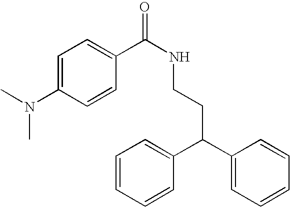

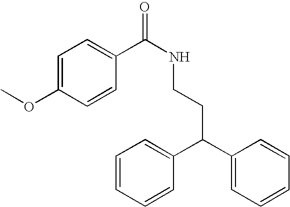

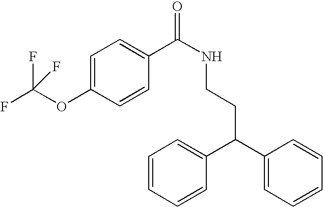

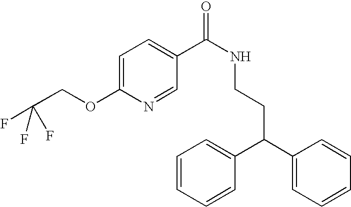

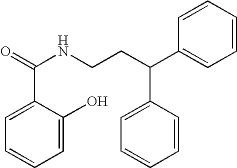

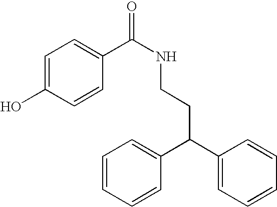

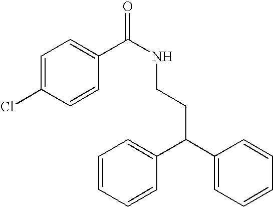

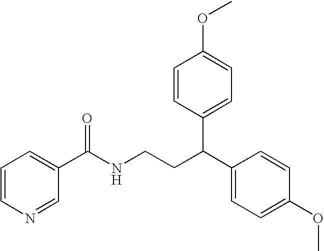

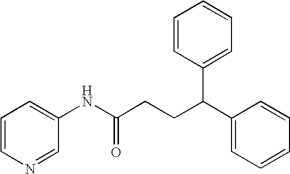

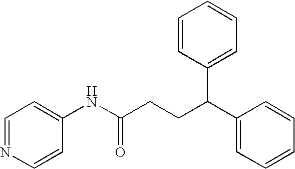

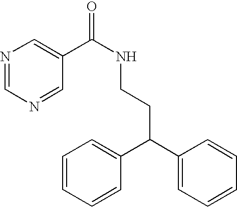

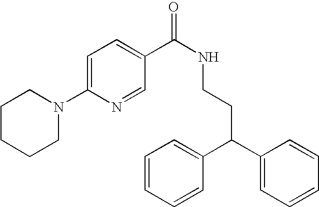

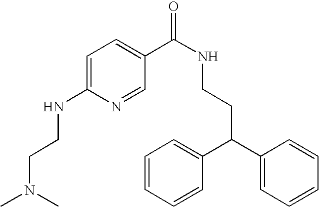

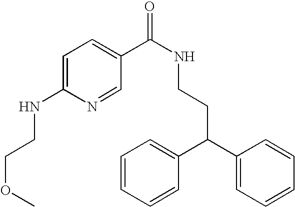

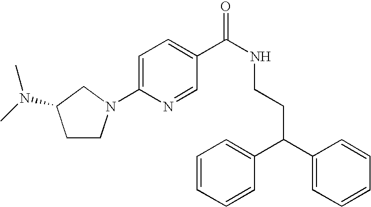

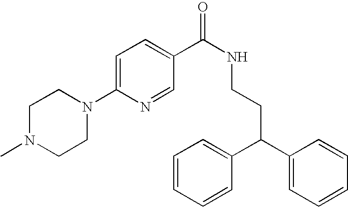

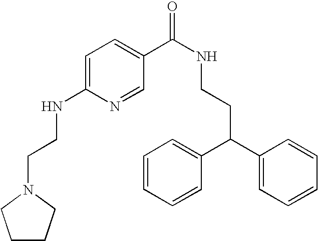

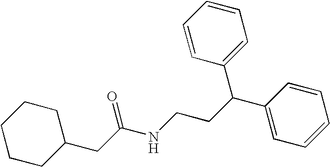

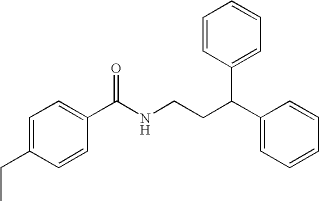

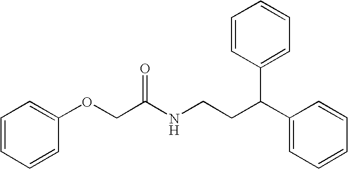

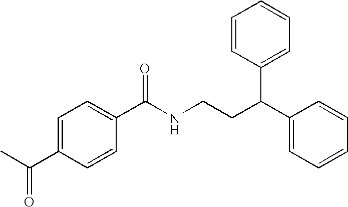

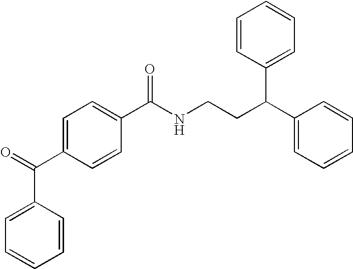

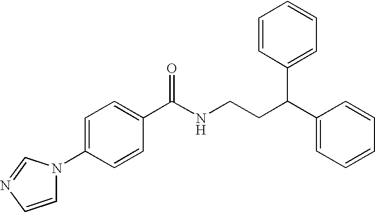

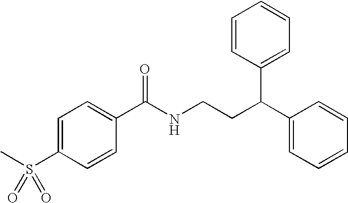

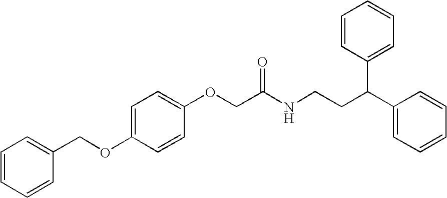

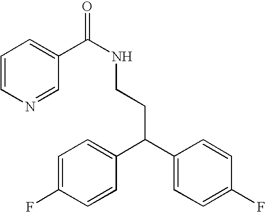

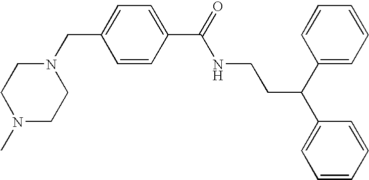

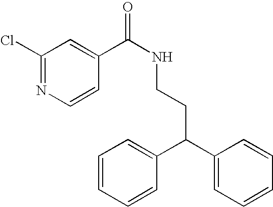

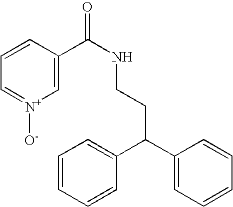

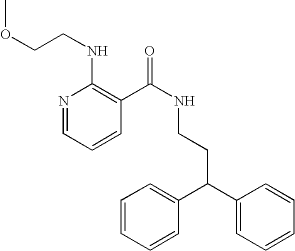

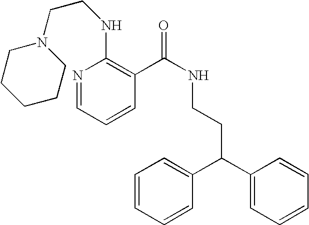

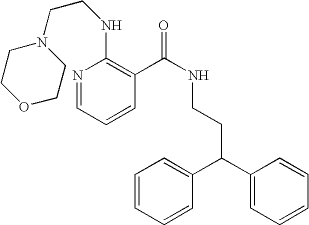

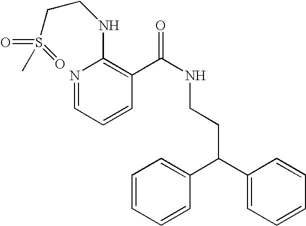

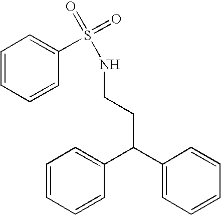

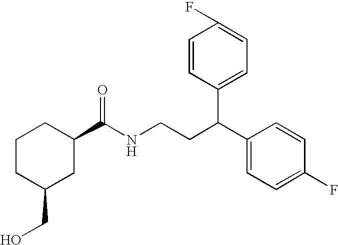

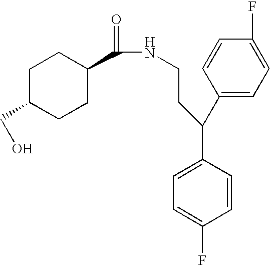

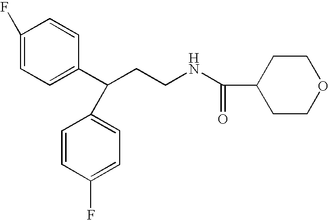

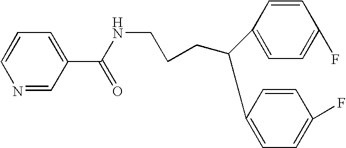

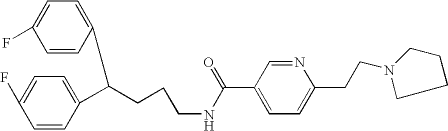

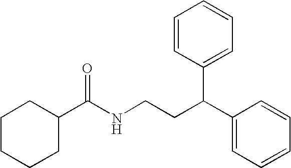

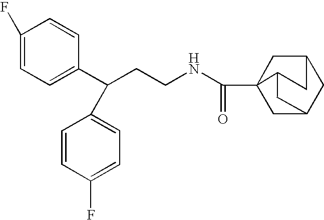

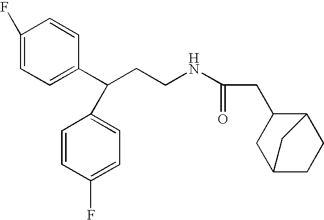

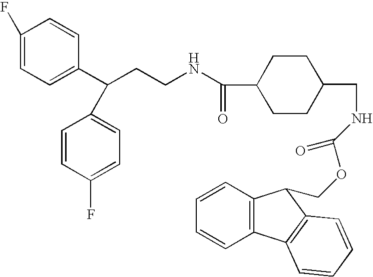

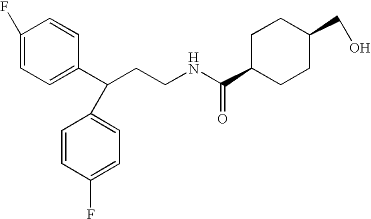

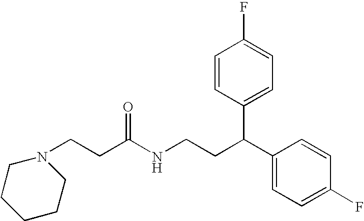

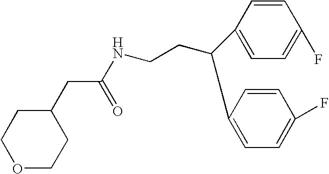

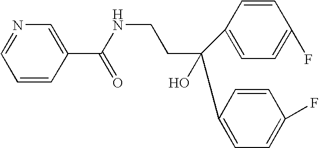

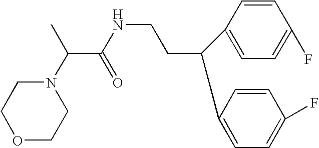

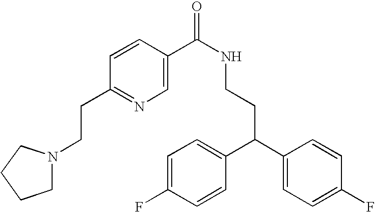

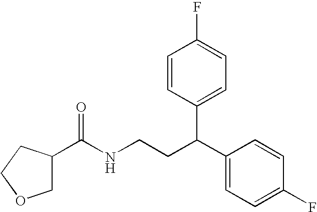

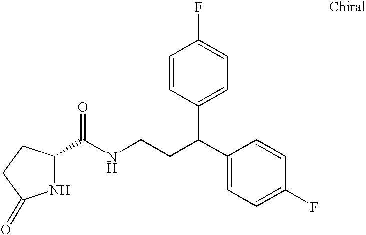

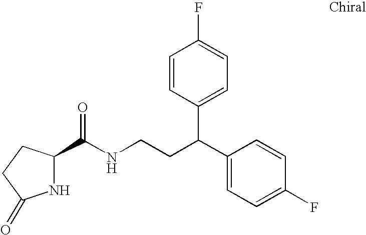

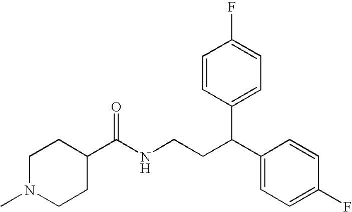

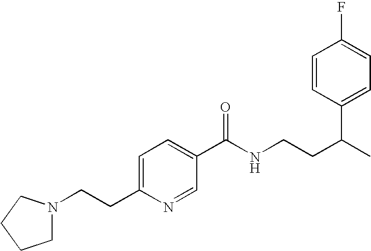

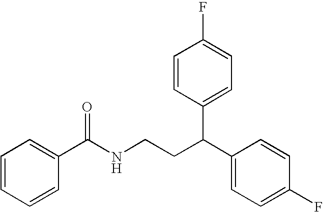

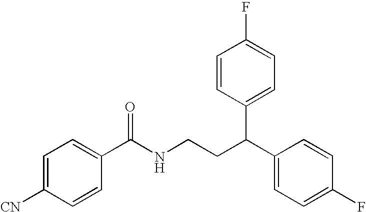

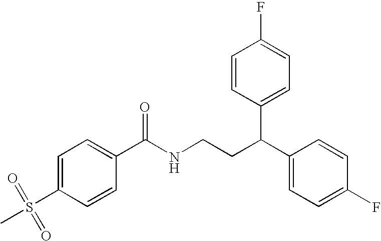

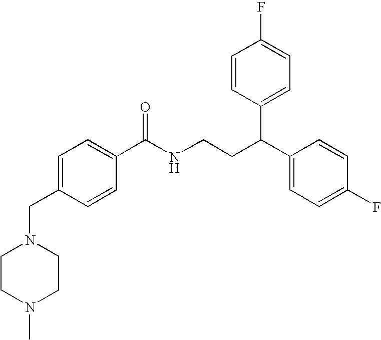

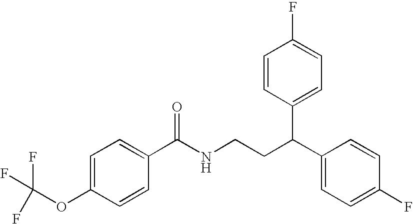

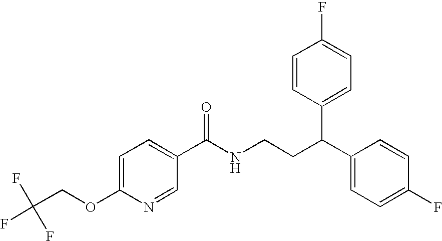

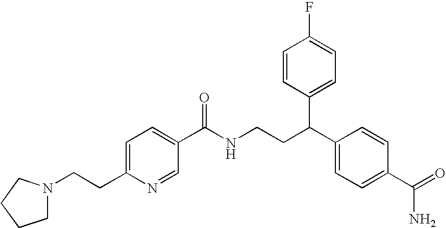

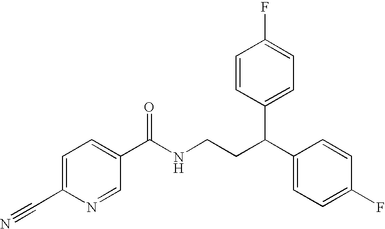

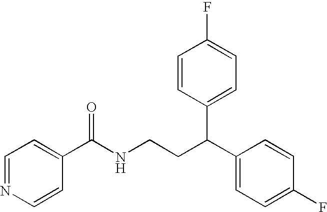

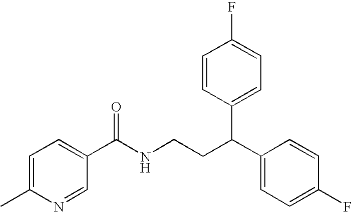

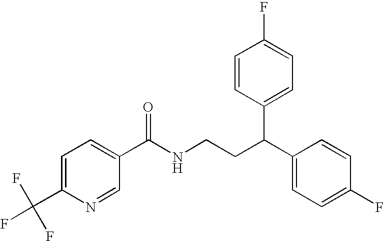

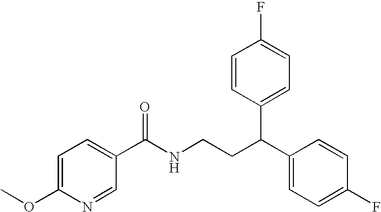

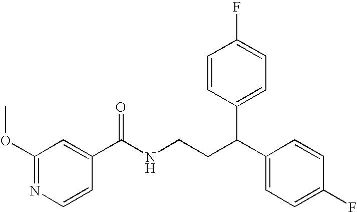

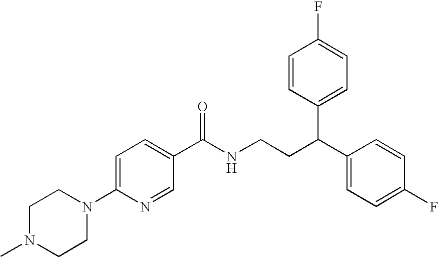

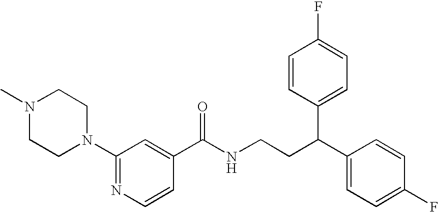

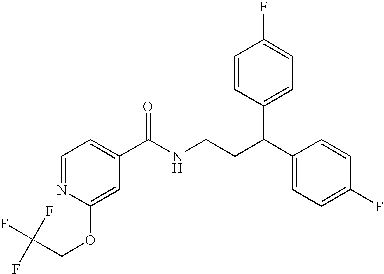

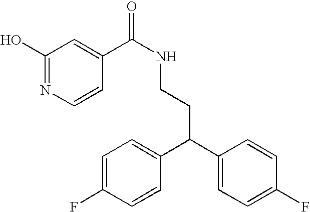

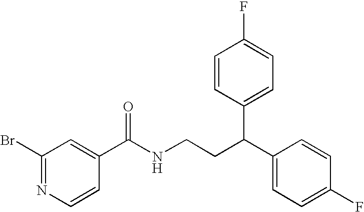

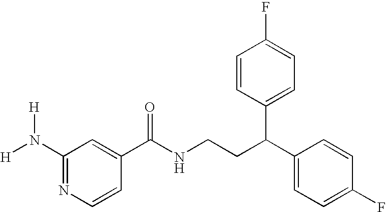

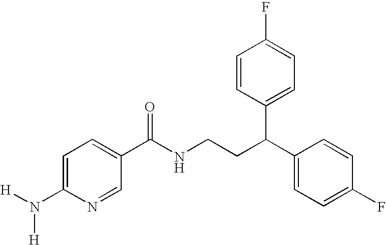

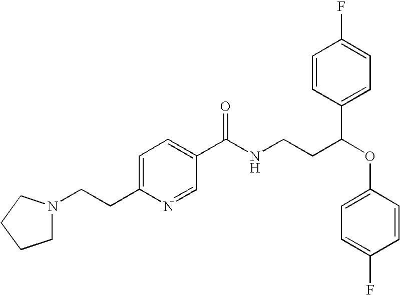

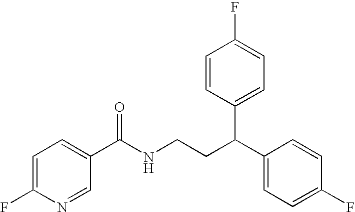

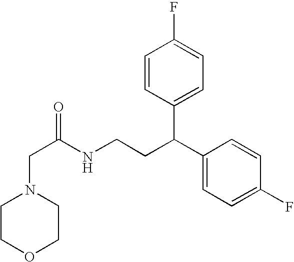

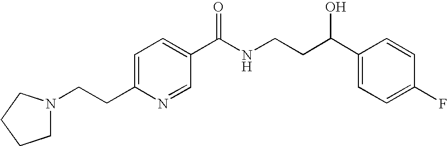

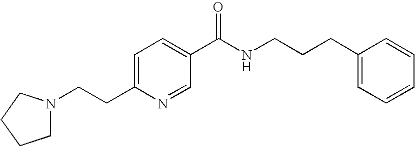

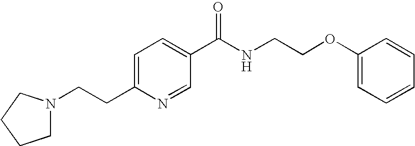

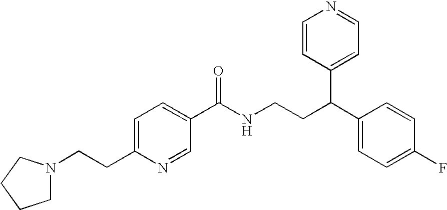

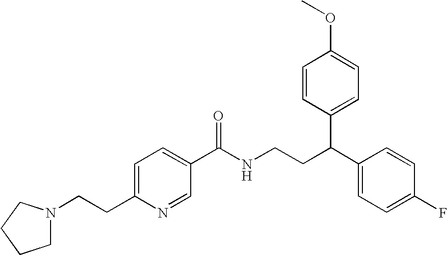

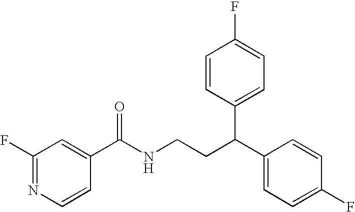

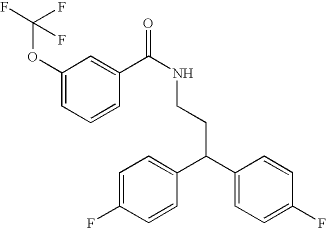

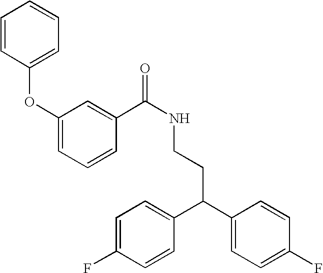

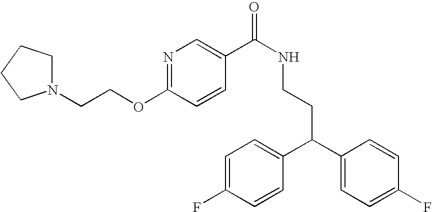

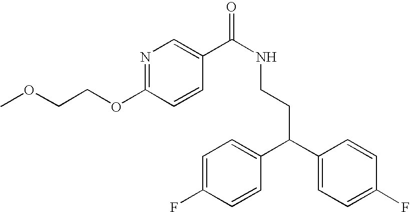

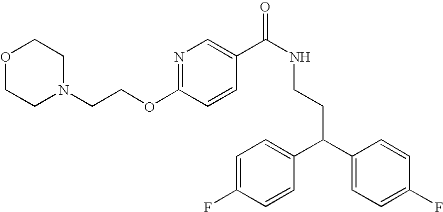

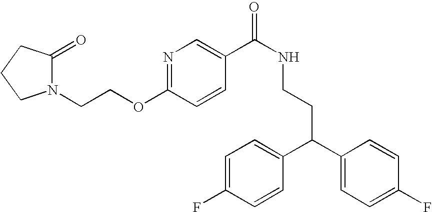

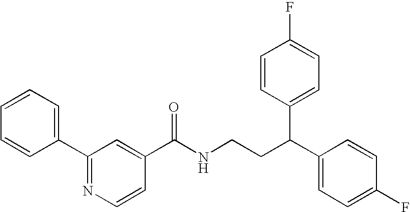

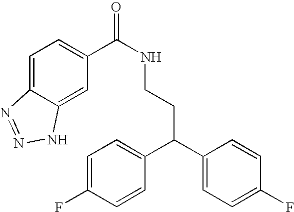

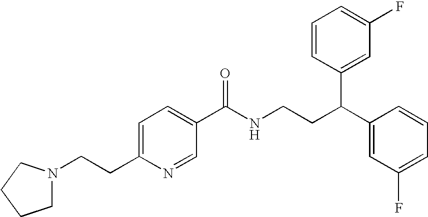

or the pharmaceutically acceptable salts thereof.

In all the compounds disclosed hereinabove in this application, in the event the nomenclature is in conflict with the structure, it shall be understood that the compound is defined by the structure.

The invention includes the use of any compounds of described above containing one or more asymmetric carbon atoms may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

Some of the compounds of formula (I) can exist in more than one tautomeric form. The invention includes methods using all such tautomers.

All terms as used herein in this specification, unless otherwise stated, shall be understood in their ordinary meaning as known in the art. For example, C₁₋₄alkoxy includes the organic radical C₁₋₄alkyl with a terminal oxygen, such as methoxy, ethoxy, propoxy, butoxy.

All organic radicals: alkyl, alkenyl and alkynyl groups, or such groups which are incorporated in other radicals such as acyl and alkoxy, shall be understood as being branched or unbranched where structurally possible and unless otherwise specified, and may be partially or fully halogenated.

The term “lower” referred to above and hereinafter in connection with organic radicals or compounds respectively defines such as branched or unbranched with up to and including 7, preferably up to and including 4 and advantageously one or two carbon atoms.

A cyclic group shall be understood to mean carbocycle, heterocycle or heteroaryl, each may be partially or fully halogenated.

An acyl group is a radical defined as —C(O)—R, where R is an organic radical or a cyclic group. Acyl represents, for example, carbocyclic or heterocyclic aroyl, cycloalkylcarbonyl, (oxa or thia)-cycloalkylcarbonyl, lower alkanoyl, (lower alkoxy, hydroxy or acyloxy)-lower alkanoyl, (mono- or di-carbocyclic or heterocyclic)-(lower alkanoyl or lower alkoxy-, hydroxy- or acyloxy-substituted lower alkanoyl), or biaroyl.

Carbocycles include hydrocarbon rings containing from three to fourteen carbon atoms. These carbocycles may be either aromatic either aromatic or non-aromatic ring systems. The non-aromatic ring systems may be mono- or polyunsaturated, monocyclic, bicyclic or tricyclic and may be bridged. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, benzyl, indanyl, indenyl, benzocyclobutanyl, dihydronaphthyl, tetrahydronaphthyl, naphthyl, decahydronaphthyl, benzocycloheptanyl, adamantyl, norbornyl, fluorene, and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms chosen from nitrogen, oxygen and sulfur. The heterocycle may be attached by any atom of the cycle, which results in the creation of a stable structure. Unless otherwise stated, heterocycles include but are not limited to, for example pyrrolidinyl, pyrrolinyl, morpholinyl, thiomorpholinyl, thiomorpholinyl sulfoxide, thiomorpholinyl sulfone, dioxalanyl, piperidinyl, piperazinyl, tetrahydrofuranyl, tetrahydropyranyl, tetrahydrofuranyl, 1,3-dioxolanone, 1,3-dioxanone, 1,4-dioxanyl, piperidinonyl, tetrahydropyrimidonyl, pentamethylene sulfide, pentamethylene sulfoxide, pentamethylene sulfone, tetramethylene sulfide, tetramethylene sulfoxide and tetramethylene sulfone.

The term “heteroaryl” shall be understood to mean an aromatic 5-8 membered monocyclic or 8-11 membered bicyclic ring containing 1-4 heteroatoms such as N,O and S. Unless otherwise stated, such heteroaryls include aziridinyl, thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl, naphthyridinyl, indazolyl, triazolyl, pyrazolo[3,4-b]pyrimidinyl, purinyl, pyrrolo[2,3-b]pyridinyl, pyrazolo[3,4-b]pyridinyl, tubercidinyl, oxazo[4,5-b]pyridinyl and imidazo[4,5-b]pyridinyl.

The term “heteroatom” as used herein shall be understood to mean atoms other than carbon such as oxygen, nitrogen, sulfur and phosphorous.

As used herein, “nitrogen” and “sulfur” include any oxidized form of nitrogen and sulfur and the quaternized form of any basic nitrogen. All heteroatoms in open chain or cyclic radicals include all oxidized forms.

In all alkyl groups or carbon chains one or more carbon atoms can be optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain. Such groups can be substituted as herein above described by groups such as oxo to result in defintions such as but not limited to: alkoxycarbonyl, acyl, amido and thioxo.

The term “aryl” as used herein shall be understood to mean aromatic carbocycle or heteroaryl as defined herein. Each aryl or heteroaryl unless otherwise specified includes it's partially or fully hydrogenated derivative and/or is partially or fully halogenated. For example, quinolinyl may include decahydroquinolinyl and tetrahydroquinolinyl, naphthyl may include it's hydrogenated derivatives such as tetrahydranaphthyl. Other partially or fully hydrogenated derivatives of the aryl and heteroaryl compounds described herein will be apparent to one of ordinary skill in the art.

The term “halogen” as used in the present specification shall be understood to mean bromine, chlorine, fluorine or iodine, preferably fluorine. The definitions “partially or fully halogenated”; partially or fully fluorinated; “substituted by one or more halogen atoms”, includes for example, mono, di or tri halo derivatives on one or more carbon atoms. For alkyl, a nonlimiting example would be —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The invention includes pharmaceutically acceptable derivatives of compounds of formula (I). A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the formula (I).

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C₁-C₄ alkyl)₄⁺ salts.

In addition, within the scope of the invention is use of prodrugs of compounds of the formula (I). Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed hereinabove, thereby imparting the desired pharmacological effect.

The compounds described herein are either commercially available or can be made by methods and any necessary intermediates well known in the art.

In order that this invention be more fully understood, the following examples are set forth. These examples are for the purpose of illustrating preferred embodiments of this invention, and are not to be construed as limiting the scope of the invention in any way.

The examples which follow are illustrative and, as recognized by one skilled in the art, particular reagents or conditions could be modified as needed for individual compounds without undue experimentation. Starting materials used in the scheme below are either commercially available or easily prepared from commercially available materials by those skilled in the art.

### General Synthetic Methods

General Synthetic Methods for Making Compounds of Formula (I), (II), and (III) The invention also provides processes for making compounds of Formula (I), (II) and (III). In all schemes, unless specified otherwise, A, Ar₁, Ar₂, Ar₃, L, n, W, X, X₁, X₂, Y and Z in the formulas below shall have the meaning of A, Ar₁, Ar₂, Ar₃, L, n, W, X, X₁, X₂, Y and Z in Formula (I), (II) and (III) of the invention described herein above.

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. Typically, reaction progress may be monitored by thin layer chromatography (TLC), if desired, and intermediates and products may be purified by chromatography on silica gel and/or by recrystallization.

The appropriately substituted starting materials and intermediates used in the preparation of compounds of the invention are either commercially available or readily prepared by methods known in the literature to those skilled in the art, and are illustrated in the synthetic examples below.

Compounds of Formula (I), (II), and (III) may be synthesized by the method illustrated in Scheme 1

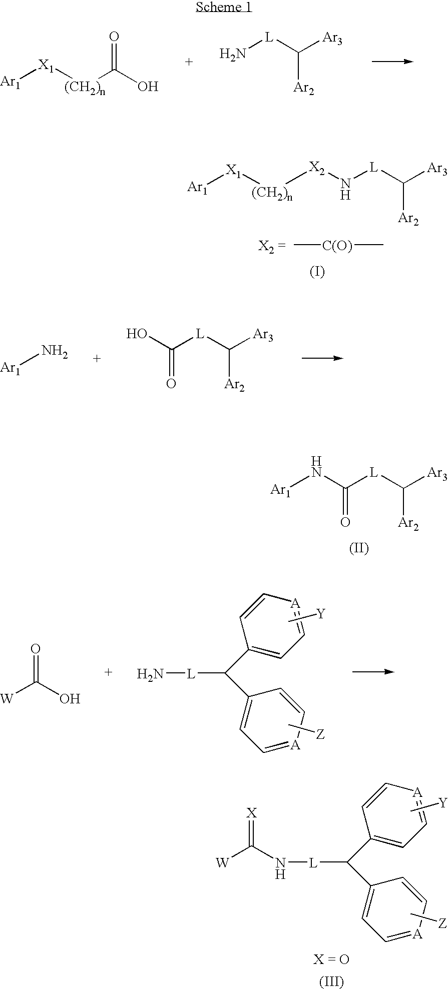

Amide coupling of the carboxylic acid with the desired amine provides the desired compound of formula (I), (II) or (III). Standard peptide coupling reactions known in the art (see for example M. Bodanszky, 1984, The Practice of Peptide Synthesis, Springer-Verlag) may be employed in these syntheses. An example of suitable coupling conditions is treatment of a solution of the carboxylic acid in a suitable solvent such as DMF with EDC, HOBT, and a base such as diisopropylethylamine, followed by the desired amine. Further modification of the initial product of formula (I), (II) and (III) by methods known in the art and illustrated in the Examples below, may be used to prepare additional compounds of this invention.

Alternatively, reaction of the carboxylic acid with reagents such as oxalyl chloride provides the corresponding acid chloride. Reaction of the acid chloride with the desired amine in a suitable solvent provides the compound of formula (I), (II) or (III).

The appropriately substituted starting materials and intermediates used in the preparation of compounds of the invention are either commercially available or readily prepared by methods known in the literature to those skilled in the art, and are illustrated in the synthetic examples below.

### EXAMPLE 1

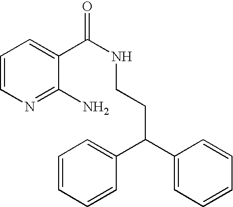

### 2-Amino-N-(3,3-diphenyl-propyl)-nicotinamide

To a solution of 2-amino-nicotinic acid (0.065 g, 0.473 mmol) in dimethylformamide (4 mL) is added 3,3-diphenyl-propylamine (0.100 g, 0.473 mmol), followed by the addition of 1-hydroxybenzotriazole (HOBT) (0.127 g, 0.946 mmol), 1-[3-(dimethylamino)propyl]-3-ethylcarbodiimde hydrochloride (EDC) (0.180 g, 0.946 mmol), and diisopropylethylamine (0.247 mL, 1.419 mmol). The reaction is stirred overnight. The mixture is diluted with water and the product is extracted using dichloromethane. The organic phase is passed through a cartridge containing silica gel and magnesium sulfate. The resulting solution is evaporated in vacuo to provide the desired product (0.109 g, 69.5%). LCMS: 332.52 (M+H⁺).

### EXAMPLE 2

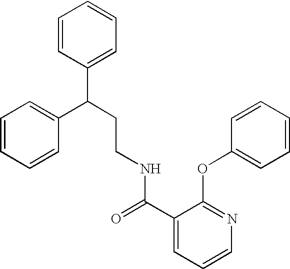

### N-(3,3-Diphenyl-propyl)-2-phenoxy-nicotinamide

The title compound is prepared and purified using the procedure from Example 1, starting from 2-phenoxy nicotinic acid (0.102 g, 0.473 mmol), to provide the desired product (0.176 g, 91.1%). LCMS: 409.46 (M+H⁺).

### EXAMPLE 3

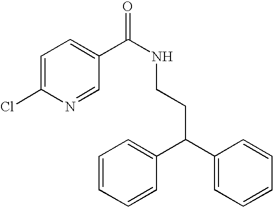

### 6-Chloro-N-(3,3-diphenyl-propyl)-nicotinamide

The title compound is prepared and purified using the procedure from Example 1, starting from 6-chloro nicotinic acid (0.075 g, 0.473 mmol), to provide the desired product (0.161 g, 97.0%). LCMS: 351.47 (M+H⁺).

### EXAMPLE 4

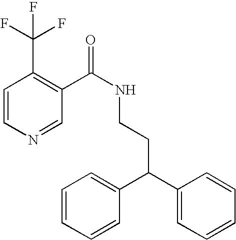

### N-(3,3-Diphenyl-propyl)-4-trifluoromethyl-nicotinamide

The title compound is prepared and purified using the procedure from Example 1, starting from 4-trifluoromethyl-nicotinic acid (0.090 g, 0.473 mmol), to provide the desired product (0.150 g, 75.0%). LCMS: 385.44 (M+H⁺).

### EXAMPLE 5

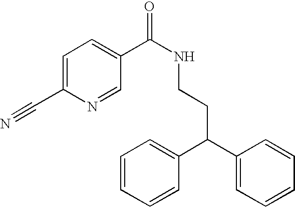

### 6-Cyano-N-(3,3-diphenyl-propyl)-nicotinamide

The title compound is prepared and purified using the procedure from Example 1, starting from 6-cyano-nicotinic acid (0.020 g, 0.135 mmol), to provide the desired product (0.043 g, 93.3%). LCMS: 342.53 (M+H⁺).

### EXAMPLE 6

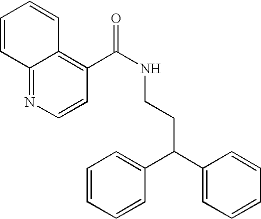

### Quinoline-4-carboxylic acid (3,3-diphenyl-propyl)-amide

The title compound is prepared and purified using the procedure from Example 1, starting from quinoline-4-carboxylic acid (0.058 g, 0.473 mmol), to provide the desired product (0.093 g, 62.7%). LCMS: 367.52 (M+H⁺).

### EXAMPLE 7

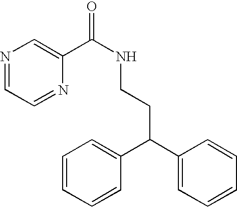

### Pyrazine-2-carboxylic acid (3,3-diphenyl-propyl)-amide

The title compound is prepared and purified using the procedure from Example 1, starting from pyrazine-2-carboxylic acid (0.061 g, 0.492 mmol). The resulting product is dissolved in dichloromethane and passed through TMA-carbonate cartridge and evaporated in vacuo to provide the desired product (0.010 g, 6.9%). LCMS: 318.44 (M+H⁺).

### EXAMPLE 8

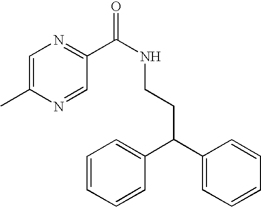

### 5-Methyl-pyrazine-2-carboxylic acid (3,3-diphenyl-propyl)-amide

The title compound is prepared and purified using the procedure from Example 1, starting from 5-methyl-pyrazine-2-carboxylic acid (0.065 g, 0.473 mmol), to provide the desired product (0.016 g, 10.2%). LCMS: 332.52 (M+H⁺).

### EXAMPLE 9

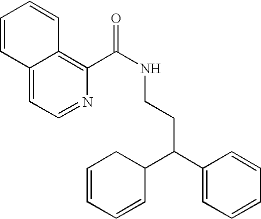

### Isoguinoline-1-carboxylic acid (3-cyclohexa-2,4-dienyl-3-phenyl-propyl)-amide

To a solution of isoquinoline-1-carboxylic acid (0.082 g, 0.473 mmol) in DMF (4 mL) is added 3,3-diphenylpropylamine (0.100 g, 0.473 mmol), followed by the addition of HOBT (0.127 g, 0.946 mmol), EDC (0.180 g, 0.946 mmol) and diisopropylethylamine (0.247 mL, 1.419 mmol). The reaction is stirred overnight. The mixture is diluted with water whereupon a solid forms. The solid is filtered off and then dissolved in dichloromethane. The resulting solution is then evaporated in vacuo to provide the desired product (0.020 g, 10.0%). LCMS: 367.51 (M+H⁺).

### EXAMPLE 10

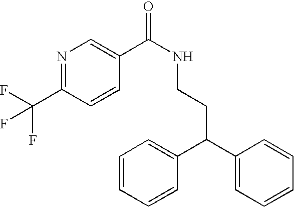

### N-(3,3-Diphenyl-propyl)-6-trifluoromethyl-nicotinamide

The title compound is prepared and purified using the procedure from Example 1, starting from 6-trifluoromethyl-nicotinic acid (0.090 g, 0.473 mmol), to provide the desired product (0.093 g, 62.7%). LCMS: 385.44 (M+H⁺).

### EXAMPLE 11

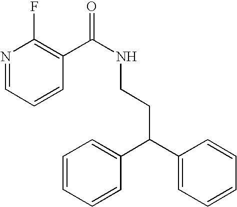

### N-(3,3-Diphenyl-propyl)-2-fluoro-nicotinamide

The title compound is prepared and purified using the procedure from Example 1, starting from 2-fluoro nicotinic acid (0.067 g, 0.473 mmol), to provide the desired product (0.109 g, 68.9%). LCMS: 335.49 (M+H⁺).

### EXAMPLE 12

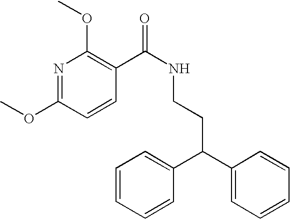

### N-(3,3-Diphenyl-propyl)-2,6-dimethoxy-nicotinamide

The title compound is prepared and purified using the procedure from Example 1, starting from 2,6-dimethoxy-nicotinic acid (0.087 g, 0.473 mmol), to provide the desired product (0.116 g, 65.1%). LCMS: 377.48 (M+H⁺).

### EXAMPLE 13

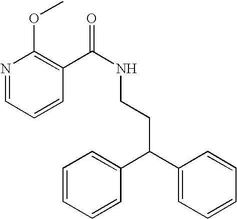

### N-(3,3-Diphenyl-propyl)-2-methoxy-nicotinamide

The title compound is prepared and purified using the procedure from Example 1, starting from 2-methoxy-nicotinicacid (0.072 g, 0.473 mmol), to provide the desired product (0.111 g, 67.7%). LCMS: 347.52 (M+H⁺).

### EXAMPLE 14

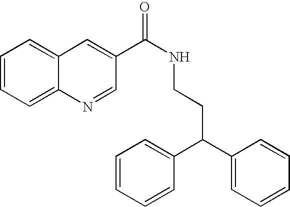

### Quinoline-3-carboxylic acid (3,3-diphenyl-propyl)-amide

The title compound is prepared and purified using the procedure from Example 1, starting from quinoline-3-carboxylic acid (0.082 g, 0.473 mmol), to provide the desired product (0.064 g, 36.9%). LCMS: 367.51 (M+H⁺).

### EXAMPLE 15

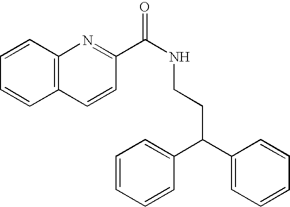

### Quinoline-2-carboxylic acid (3,3-diphenyl-propyl)-amide

The title compound is prepared and purified using the procedure from Example 1, starting from isoquinoline-1-carboxylic acid (0.082 g, 0.473 mmol), to provide the desired product (0.060 g, 3.7%). LCMS: 367.52 (M+H⁺).

### EXAMPLE 16

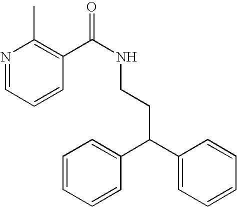

### N-(3,3-Diphenyl-propyl)-2-methyl-nicotinamide

The title compound is prepared and purified using the procedure Example 7, starting from 2-methyl-nicotinic acid (0.065 g, 0.473 mmol), to provide the desired product (0.097 g, 62.1%). LCMS: 331.55(M+H⁺).

### EXAMPLE 17

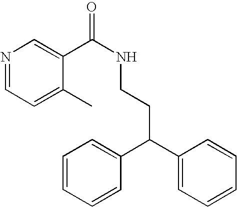

### N-(3,3-Diphenyl-propyl)-4-methyl-nicotinamide

The title compound is prepared and purified using the procedure from Example 7, starting from 4-methyl-nicotinic acid hydrochloride salt, (0.082 g, 0.473 mmol), to give the desired product (0.051 g, 25.4%). LCMS: 331.52 (M+H⁺).

### EXAMPLE 18

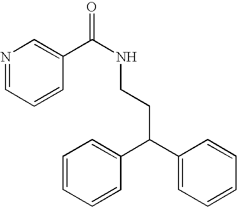

### N-(3,3-Diphenyl-propyl)-nicotinamide

The title compound is prepared and purified using the procedure from Example 17, starting from nicotinic acid, (0.058 g, 0.473 mmol), to provide the desired product (0.109 g, 73.3%). LCMS: 317.52 (M+H⁺).

### EXAMPLE 19

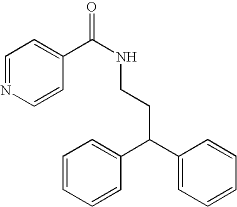

### N-(3,3-Diphenyl-propyl)-isonicotinamide

The title compound is prepared and purified using the procedure from Example 7, starting from iso-nicotinic acid (0.058 g, 0.473 mmol), to provide the desired product (0.094 g, 62.7%). LCMS: 317.51 (M+H⁺).

### EXAMPLE 20

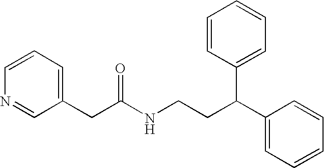

### N-(3,3-Diphenyl-propyl)-2-pyridin-3-yl-acetamide

The title compound is prepared using the procedure from Example 1, starting from pyridine-3-yl-acetic acid; hydrochloride salt (0.200 g, 1.15 mmol). Water is then is added to the mixture and it is allowed to stand for few hours whereupon a solid forms. The solid is filtered off and dried in vacuo to provide the desired product (0.309 g, 81.2%). LCMS: 331.38 (M+H⁺).

### EXAMPLE 21

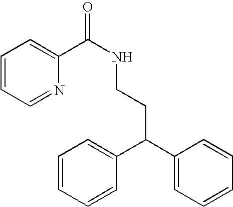

### Pyridine-2-carboxylic acid (3,3-diphenyl-propyl)-amide

The title compound is prepared and purified using the procedure from Example 7, starting from pyridine-2-carboxylic acid (0.058 g, 0.473 mmol), to provide the desired product (0.016 g, 11.0%). LCMS: 317.44 (M+H⁺).

### EXAMPLE 22

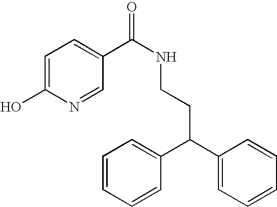

### N-(3,3-Diphenyl-propyl)-6-hydroxy-nicotinamide

The title compound is prepared using the procedure from Example 1, starting from 6-hydroxy-nicotinic acid (0.066 g, 0.473 mmol). The resulting compound is purified (flash chromatography, 2-10% MeOH in dichloromethane) to provide the desired product (0.041 g, 26.3%). LCMS: 333.58 (M+H⁺).

### EXAMPLE 23

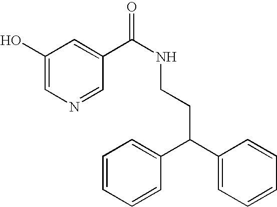

### N-(3,3-Diphenyl-propyl)-5-hydroxy-nicotinamide

The title compound is prepared using the procedure from Example 1, starting from 5-hydroxy-nicotinic acid (0.066 g, 0.473 mmol). The resulting compound is purified (flash chromatography, 2-10% MeOH in dichloromethane) to provide the desired product (0.063 g, 40.3%). LCMS: 333.05 (M+H⁺).

### EXAMPLE 24

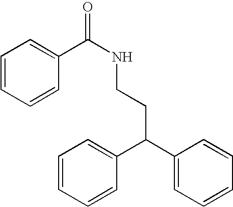

### N-(3,3-Diphenyl-propyl)-benzamide

The title compound is prepared and purified using the procedure from Example 7, starting from benzoic acid (0.100 g, 0.819 mmol), to provide the desired product (0.231 g, 89.5%). LCMS: 316.36 (M+H⁺).

### EXAMPLE 25

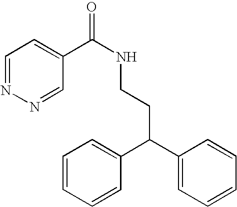

### Pyridazine-4-carboxylic acid (3,3-diphenyl-propyl)-amide

The title compound is prepared and purified using the procedure from Example 7, starting from pyrazine carboxylic acid (0.117 g, 0.946 mmol), to provide the desired product (0.223 g, 74.3%). LCMS: 318.34 (M+H⁺).

### EXAMPLE 26

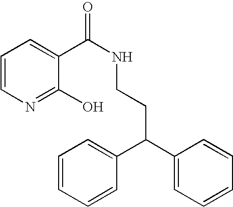

### N-(3,3-Diphenyl-propyl)-2-hydroxy-nicotinamide

The title compound is prepared using the procedure from Example 1, starting from 2-hydroxy nicotinic acid (0.100 g, 0.719 mmol). The resulting compound is dissolved in dichloromethane and passed through TMA-carbonate silica cartridge, evaporated and then crystallized from Et₂O/few drops of dichloromethane, to provide the desired product (0.014 g, 5.9%). LCMS: 333.34 (M+H⁺).

### EXAMPLE 27

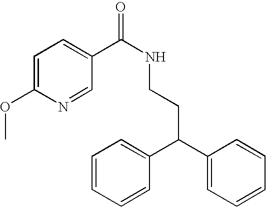

### N-(3,3-Diphenyl-propyl)-6-methoxy-nicotinamide

The title compound is prepared and purified using the procedure from Example 7, starting from 6-methoxy nicotinic acid (0.100 g, 0.653 mmol), to give the desired product (0.178 g, 78.7%). LCMS: 347.35 (M+H⁺).

### EXAMPLE 28

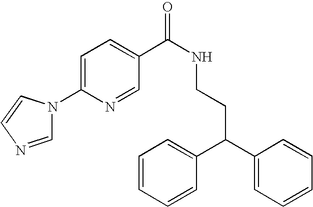

### N-(3,3-Diphenyl-propyl)-6-imidazol-1-yl-nicotinamide

The title compound is prepared using the procedure from Example 7, starting from 6-imidazol-1-yl-nicotinic acid (0.100 g, 0.529 mmol), to provide the desired product (0.052 g, 25.7%). LCMS: 383.32 (M+H⁺).

### EXAMPLE 29

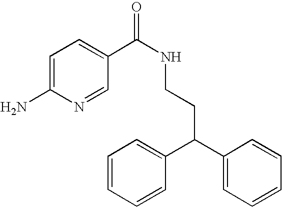

### 6-Amino-N-(3,3-diphenyl-propyl)-nicotinamide

The title compound is prepared using the procedure from Example 7, starting from 6-amino-nicotinic acid

(0.100 g, 0.724 mmol), to provide the desired product (0.073 g, 30.4%). LCMS: 332.35 (M+H⁺).

### EXAMPLE 30

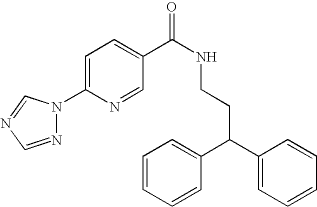

### N-(3,3-Diphenyl-propyl)-6-[1,2,4]triazol-1-yl-nicotinamide

The title compound is prepared and purified using the procedure from example 7, starting from 6-[1,2,4]triazole-1-yl nicotinic acid (0.100 g, 0.526 mmol), to provide the desired product (0.091 g, 45.1%). LCMS: 384.31 (M+H⁺).

### EXAMPLE 31

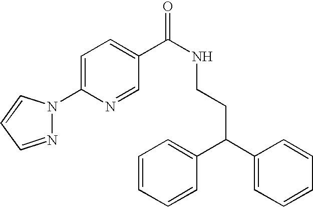

### N-(3,3-Diphenyl-propyl)-6-pyrazol-1-yl-nicotinamide

The title compound is prepared using the procedure from Example 9, starting from 6-pyrazole-1-yl-nicotinic acid (0.100 g, 0.529 mmol), to provide the desired product (0.141 g, 69.7%). LCMS: 383.32 (M+H⁺).

### EXAMPLE 32

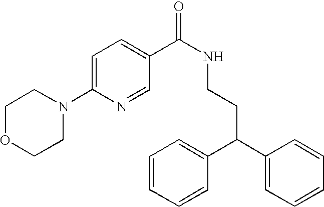

### N-(3,3-Diphenyl-propyl)-6-morpholin-4-yl-nicotinamide

The title compound is prepared using the procedure from Example 1, starting from 6-morpholine-4-yl-nicotinic acid (0.100 g, 0.480 mmol). The mixture is diluted with water, and after couple of hours a viscous liquid forms at the bottom of the vial. The water layer is removed and the oil is washed several times with water and then ether. Dichloromethane and Et₂O (2-4 mL) are added to that and the solution is evaporated in vacuo, to provide the desired product (0.125 g, 64.9%). LCMS: 402.34 (M+H⁺).

### EXAMPLE 33

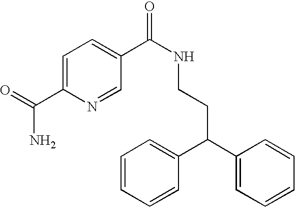

### Pyridine-2,5-dicarboxylic acid 2-amide 5-[(3,3-diphenyl-propyl)-amide]

The product from Example 5 (0.100 g, 0.293 mmol) is added to a mixture of ethanol and water (2.5 mL, 1:1). Sodium perborate is then added and the mixture is placed in a microwave for 4 minutes at 100° C. The resulting white solid in the mixture is filtered off and dried in vacuo, to provide the desired product (0.050 g, 47.5%). LCMS: 360.33 (M+H⁺).

### EXAMPLE 34

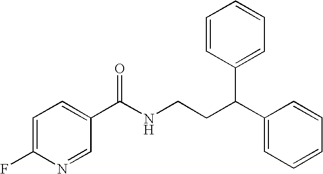

### N-(3,3-Diphenyl-propyl)-6-fluoro-nicotinamide

The title compound is prepared and purified using the procedure from Example 9, starting from 6-fluoro-nicotinic acid (0.068 g, 0.480 mmol), to provide the desired product (0.130 g, 81.0%). LCMS: 335.4

### EXAMPLE 35

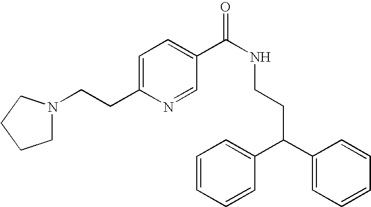

### N-(3,3-Diphenyl-propyl)-6-(2-pyrrolidin-1-yl-ethyl)-nicotinamide

The title compound is prepared and purified using the procedure from Example 32, starting from 6-(2-pyrrolidine-1-yl-ethyl)-nicotinic acid (0.105 g, 0.480 mmol), to provide the desired product (0.085 g, 42.8%). LCMS: 414.38 (M+H⁺).

### EXAMPLE 36

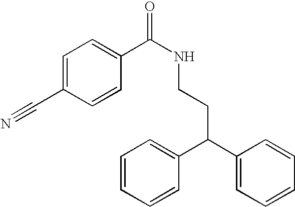

### 4-Cyano-N-(3,3-diphenyl-propyl)-benzamide

The title compound is prepared and purified using the procedure from Example 32, starting from 4-cyano-benzoic acid (0.071 g, 0.480 mmol), to provide the desired the product (0.092 g, 56.3%). LCMS: 341.33 (M+H⁺).

### EXAMPLE 37

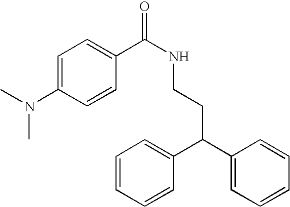

### 4-Dimethylamino-N-(3,3-diphenyl-propyl)-benzamide

The title compound is prepared and purified using the procedure from Example 32, starting from 6-dimethylamino-nicotinic acid (0.079 g, 0.480 mmol), to provide the desired product (0.152 g, 89.5%). LCMS: 359.36 (M+H⁺).

### EXAMPLE 38

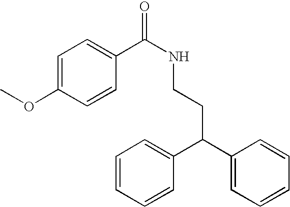

### N-(3,3-Diphenyl-propyl)-4-methoxy-benzamide

The title compound is prepared and purified using the procedure from Example 32, starting from 4-methoxy-benzoic acid (0.073 g, 0.480 mmol), to provide the desired product (0.080 g, 48.2%). LCMS: 346.34 (M+H⁺).

### EXAMPLE 39

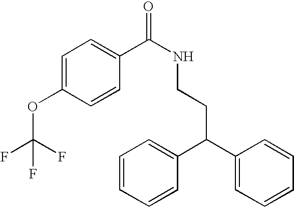

### N-(3,3-Diphenyl-propyl)-4-trifluoromethoxy-benzamide

The title compound is prepared and purified using the procedure from Example 32, starting from 4-trifluoromethoxy-benzoic acid (0.099 g, 0.480 mmol), to provide the desired product (0.135 g, 70.4%). LCMS: 400.26 (M+H⁺).

### EXAMPLE 40

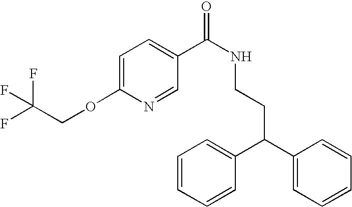

### N-(3,3-Diphenyl-propyl)-6-(2,2,2-trifluoro-ethoxy)-nicotinamide

The title compound is prepared and purified using the procedure from Example 32, starting from 6-trifluoroethoxy nicotinic acid (0.106 g, 0.480 mmol), to provide the desired product (0.115 g, 57.8%). LCMS: 415.43 (M+H⁺).

### EXAMPLE 41

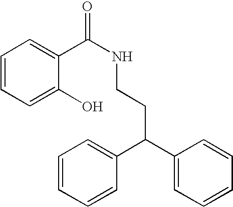

### N-(3,3-Diphenyl-propyl)-2-hydroxy-benzamide

The title compound is prepared and purified using the procedure from Example 32, starting from 2-hydroxy-benzoic acid (0.071 g, 0.480 mmol), to give the desired product (0.092 g, 56.3%). LCMS: 332.34 (M+H⁺).

### EXAMPLE 42

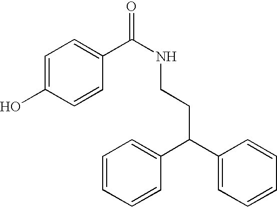

### N-(3,3-Diphenyl-propyl)-4-hydroxy-benzamide

The title compound is prepared and purified using the procedure from Example 32, starting from 4-hydroxy-benzoic acid (0.066 g, 0.480 mmol), to provide the desired product (0.056 g, 35.2%). LCMS: 332.32 (M+H⁺).

### EXAMPLE 43

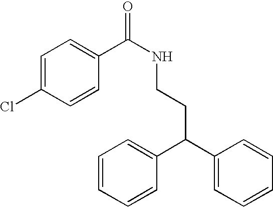

### 4-Chloro-N-(3,3-diphenyl-propyl)-benzamide

The title compound is prepared and purified using the procedure from Example 32, starting from 4-chloro-benzoic acid (0.075 g, 0.480 mmol), to provide the desired product (0.065 g, 38.7%). LCMS: 350.32 (M+H⁺).

### EXAMPLE 44

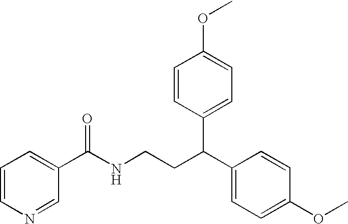

### N-[3,3-Bis-(4-methoxy-phenyl)-propyl]1-nicotinamide

### Step A: 3,3-Bis-(4-methoxy-phenyl)-acrylonitrile

To a solution of cyanomethyl-phosphonic acid diethyl ester (0.146 g, 0.826 mmol) in acetonitrile (2.5 mL), is added crushed potassium hydroxide (0.092, 1.652 mmol) and stirred for few minutes until it becomes light yellow. Bis-(4-methoxy-phenyl)-methanone (0.200 g, 0.826 mmol) is added to the mixture and it becomes dark red. The mixture is then placed in a microwave at 100° C. for 30 minutes. It is then concentrated and washed several times with ether. The ether phase is evaporated in vacuo to provide the desired product.

### Step B: 3,3-Bis-(4-methoxy-phenyl)-propionitrile

The carbonitrile product from Step A (0.315 g, 1.187 mmol) is added to the nitrogen filled flask containing palladium on carbon (10%, 0.100 g). A hydrogen balloon is attached to that and the mixture is stirred overnight. The solution is filtered and the filtrate is evaporated in vacuo to provide the desired product.

### Step C: 3,3-Bis-(4-methoxy-phenyl)-propylamine

To the solution of the product from Step B (0.336 g, 1.257 mmol) in dry THF (4 mL) at 0° C., is added LiAlH₄ in THF (1 M, 1.7 mL, 1.7 mmol) drop wise. The mixture is stirred at 0° C. for 1.5 hour and monitored by TLC which shows formation of more polar spot (5% MeOH in dichloromethane). It is allowed to come to room temperature and stirred for another 1.5 hour. Sodium bicarbonate is added to the reaction which immediately forms a solid. The mixture is passed through a layer of magnesium sulfate and the filtrate is evaporated in vacuo to provide the desired product (0.100 g, 29.3%).

### Step D: N-[3,3-Bis-(4-methoxy-phenyl)-propyl]-nicotinamide

The title compound is prepared using the procedure from Example 1, starting from nicotinic acid (0.183 g, 1.487 mmol) and the product from step C (scaled up, 0.404 g, 1.487 mmol) and purified (preparative TLC, 5% MeOH in dichloromethane), to provide desired product (0.017 g, 3.0%). LCMS: 377.34 (M+H⁺).

### EXAMPLE 45

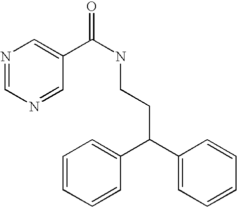

### Pyrimidine-5-carboxylic acid (3,3-diphenyl-propyl)-amide

The title compound is prepared and purified (flash chromatography, 0-5% MeOH in dichloromethane) using the procedure from Example 32, starting from pyrimidine-5-carboxylic acid (0.200 g, 1.612 mmol), to provide the desired product (0.070 g, 13.7%). LCMS: 318.02 (M+H⁺).

### EXAMPLE 46

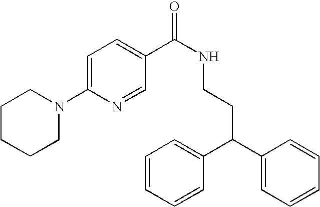

### 3,4,5,6-Tetrahydro-2H-[1,2′]bipyridinyl-5′-carboxylic acid (3,3-diphenyl-propyl)-amide

The product of Example 34 (0.050 g, 0.150 mmol) is dissolved in THF (2.5 mL) and piperidine (0.100 mL, 0.989 mmol) is added to that, followed by the addition of aqueous KOH (0.200 mL, 0.400 mmol). The mixture is placed in a microwave and heated at 90° C. for 30 minutes. Water is added to the mixture and the organic phase is extracted using dichloromethane. The dichloromethane extract is then dried over magnesium sulfate and evaporated in vacuo. The resulting product is purified (flash chromatography, 0-50% EtOAc in hexane) to provide the desired product (0.035 g, 58.4%). LCMS: 400.04 (M+H⁺).

### EXAMPLE 47

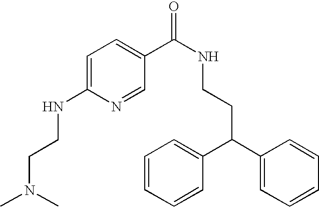

### 6-(2-Dimethylamino-ethylamino)-N-(3,3-diphenyl-propyl)-nicotinamide

The title compound is prepared and purified using the procedure from Example 46, starting from the product of Example 34 (0.050 g, 0.150 mmol) and 2-dimethyl-diaminoethyl (0.078 mL, 0.900 mmol) to provide the desired product (0.010 g, 16.9%). LCMS: 403.40 (M+H⁺).

### EXAMPLE 48

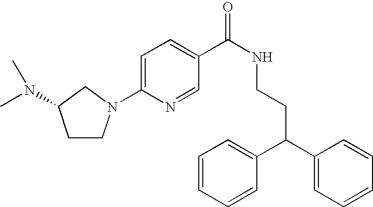

### 6-[(S)-3-Dimethylamino-pyrrolidin-1-yl]-N-(3,3-diphenyl-propyl)-nicotinamide

The title compound is prepared and purified using the procedure from Example 46, starting from N-(3,3-diphenyl propyl)-6-fluoro-nicotinamide (0.050 g, 0.150 mmol) and dimethyl-(S)-pyrrolidin-3-yl-amine (0.034 mL, 0.300 mmol), to provide the desired product (0.051 g, 79.6%). LCMS: 429.35 (M+H⁺).

### EXAMPLE 49

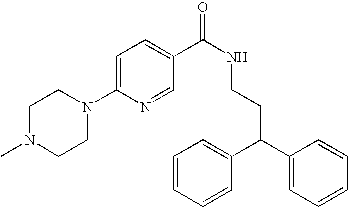

### N-(3,3-Diphenyl-propyl)-6-(4-methyl-piperazin-1-yl)-nicotinamide

The title compound is prepared and purified using the procedure from Example 46, starting from N-(3,3-diphenyl propyl)-6-fluoro-nicotinamide (0.050 g, 0.150 mmol) and 1-methyl-piperazine (0.030 mL, 0.300 mmol) to provide the desired product (0.035 g, 35.8%). LCMS: 415.36 (M+H⁺).

### EXAMPLE 50

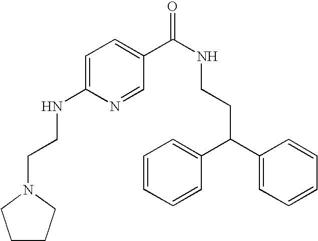

### N-(3,3-Diphenyl-propyl)-6-(2-pyrrolidin-1-yl-ethylamino)-nicotinamide

The title compound is prepared and purified using the procedure from Example 46, starting from N-(3,3-diphenyl propyl)-6-fluoro-nicotinamide carboxylic acid (0.050 g, 0.150 mmol) and 2-pyrrolidin-1-yl-ethylamine (0.034 g, 0.300 mmol), to provide the desired product (0.033 g, 51.3%). LCMS: 429.36.

### EXAMPLE 51

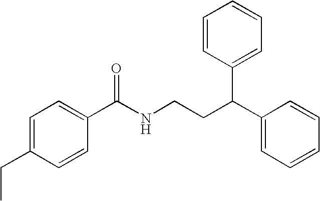

### N-(3,3-Diphenyl-propyl)-4-ethyl-benzamide

The title compound is prepared and purified using the procedure from Example 32, starting from 4-ethyl-benzoic acid (0.075 g, 0.480 mmol) to provide the desired product (0.065 g, 38.7%). LCMS: 344.48 (M+H⁺).

### EXAMPLE 52

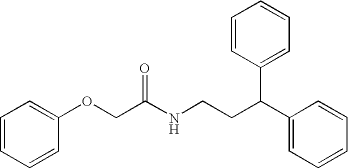

### N-(3,3-Diphenyl-propyl)-2-phenoxy-acetamide

The title compound is prepared and purified using the procedure from Example 32, starting from 4-chloro-benzoic acid (0.075 g, 0.480 mmol), to provide the desired product (0.065 g, 38.7%). LCMS: 346.46 (M+H⁺).

### EXAMPLE 53

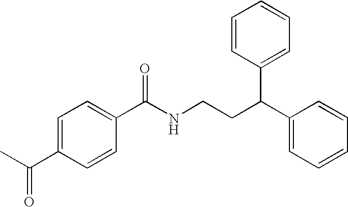

### 4-Acetyl-N-(3,3-diphenyl-propyl)-benzamide

The title compound is prepared and purified using the procedure from Example 32, starting from 4-acetyl-benzoic acid (0.075 g, 0.480 mmol), to provide the desired product (0.065 g, 38.7%). LCMS: 358.44, 715.41 (M+H⁺ and 2M+H⁺, respectively).

### EXAMPLE 54

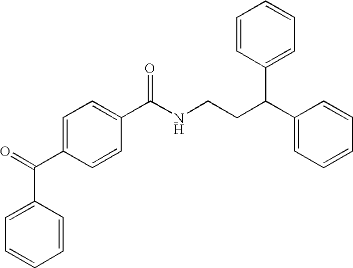

### 4-Benzoyl-N-(3,3-diphenyl-propyl)-benzamide

The title compound is prepared and purified using the procedure from Example 32, starting from 4-benzoyl-benzoic acid to provide the desired product (0.136 g, 38.7%). LCMS: 420.39 (M+H⁺).

### EXAMPLE 55

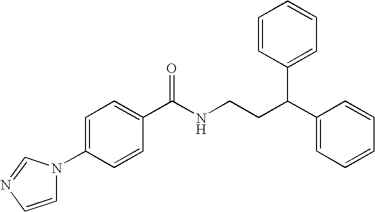

### N-(3,3-Diphenyl-propyl)-4-imidazol-1-yl-benzamide

The title compound is prepared and purified using the procedure from Example 32, starting from 4-imidazole-benzoic acid (0.075 g, 0.480 mmol), to provide the desired product (0.065 g, 38.7%). LCMS: 382.31 (M+H⁺).

### EXAMPLE 56

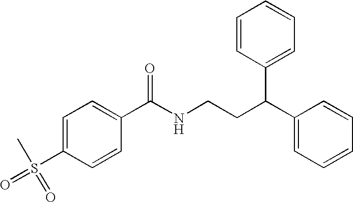

### N-(3,3-Diphenyl-propyl)-4-methanesulfonyl-benzamide

The title compound is prepared and purified using the procedure from Example 32, starting from 4-chloro-benzoic acid (0.075 g, 0.480 mmol), to provide the desired product (0.065 g, 38.7%). LCMS: 394.37 (M+H⁺).

### EXAMPLE 57

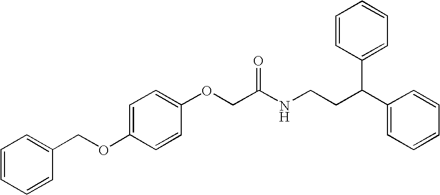

### N-(3,3-Diphenyl-propyl)-4-methanesulfonyl-benzamide

The title compound is prepared and purified using the procedure from Example 32, starting from 4-chloro-benzoic acid (0.075 g, 0.480 mmol), to provide the desired product (0.065 g, 38.7%). LCMS: 452.43 (M+H⁺).

### EXAMPLE 58

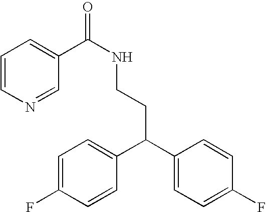

### N-[3,3-Bis-(4-fluoro-phenyl)-propyl]-nicotinamide

### Step A: 3,3-Bis-(4-fluoro-phenyl)-propylamine

The amine is made using the procedures in Example 44 Steps A, B and C starting from 4,4′-difluorobenzophenone (20.0 g, 91.6 mmol), to give the desired compound (17.0 g, 76.9%, over three steps).

### Step B: N-[3,3-Bis-(4-fluoro-phenyl)-propyl]-nicotinamide

The title compound is prepared and purified (preparative TLC, 1-10% MeOH in dichloromethane) using the procedure from Example 1, starting from nicotinic acid (0.060 g, 0.473 mmol) and 3,3-bis-(4-fluoro-phenyl)-propylamine from Step A (0.120 g, 0.487 mmol), to provide the desired product (0.025 g, 14.6%). LCMS: 353.42 (M+H⁺).

### EXAMPLE 59

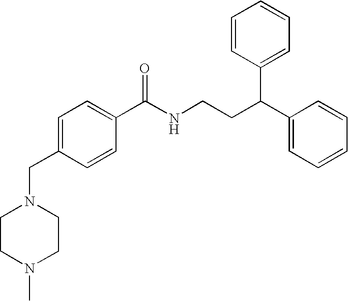

### N-(3,3-Diphenyl-propyl)-4-(4-methyl-piperazin-1-ylmethyl)-benzamide

The title compound is prepared and purified using the procedure from Example 32, starting from 4-(4-methyl-piperazine-1-ylmethyl)-benzoic acid (0.056 g, 0.237 mmol) to provide the desired product (0.030 g, 29.6%). LCMS: 428.49 (M+H⁺).

### EXAMPLE 60

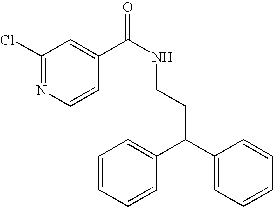

### 2-Chloro-N-(3,3-diphenyl-propyl)-isonicotinamide

The title compound is prepared and purified using the procedure from Example 32, starting from 2-chloro-isonicotinic acid (0.700 g, 4.443 mmol), to provide the desired product (1.200 g, 77.0%). LCMS: 351.0 (M+H⁺).

### EXAMPLE 61

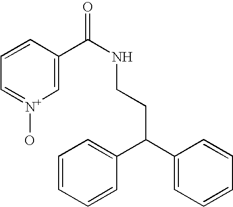

### N-(3,3-Diphenyl-propyl)-1-oxy-nicotinamide

To the solution of product from Example 18 (0.100 g, 0.316 mmol) in dichloromethane (4 mL) is added MCPBA (0.200 g, 0.263 mmol) and the mixture is stirred at room temperature for 24 hours. To the mixture is added PS-TBD to scavenge MCPBA. The solution is filtered, and the resulting solution is evaporated in vacuo and then crystallized from ether and few drops of dichloromethane, to provide the desired product (0.040 g, 38.1%). LCMS: 333.32 (M+H⁺)

### EXAMPLE 62

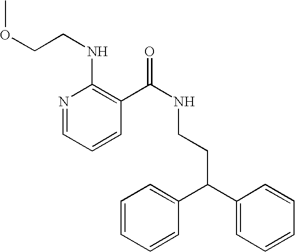

### N-(3,3-Diphenyl-propyl)-2-(2-methoxy-ethylamino)-nicotinamide

To the solution of product from Example 11 (0.050 g, 0.150 mmol) in 2.5 mL THF, is added 2-methoxy-ethylamine (0.110 g, 1.500 mmol) and the mixture is placed in a microwave and heated at 100° C. for 40 minutes. The mixture is evaporated in vacuo and to the resulting film is added ether. After 20 minutes the resulting solid is filtered off and dried in vacuo. It is then passed through para-toluene sulfonic acid-dervitized silica gel cartridge to scavenge the amine, washed with MeOH: dichloromethane (1:1) solution and evaporated in vacuo, to provide the desired product (0.050 g, 86.4%). LCMS: 390.35 (M+H⁺).

### EXAMPLE 63

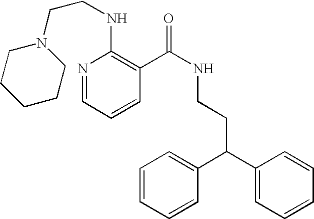

### N-(3,3-Diphenyl-propyl)-2-(2-piperidin-1-yl-ethylamino)-nicotinamide

The title compound is prepared and purified using the procedure from Example 62, starting from the product of Example 11 (0.050 g, 0.150 mmol) and 2-piperidine-1-yl-ethylamine (0.190 g, 0.150 mmol), to provide the desired product (0.010 g, 17.1%). LCMS: 443.40 (M+H⁺).

### EXAMPLE 64

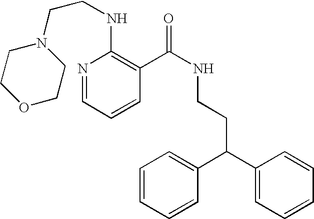

### N-(3,3-Diphenyl-propyl)-2-(2-morpholin-4-yl-ethylamino)-nicotinamide

The title compound is prepared and purified using the procedure from Example 62, starting from the product of Example 11 (0.050 g, 0.150 mmol) and 2-morpholin-4-yl-ethylamine (0.200 g, 0.150 mmol), to provide the desired product (0.030 g, 45.0%). LCMS: 445.35 (M+H⁺).

### EXAMPLE 65

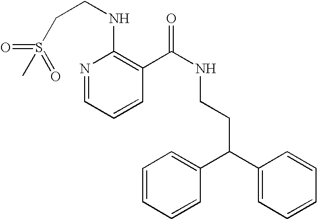

### N-(3,3-Diphenyl-propyl)-2-(2-methanesulfonyl-ethylamino)-nicotinamide

The product of Example 11 (0.050 g, 0.150 mmol) is dissolved in THF and 2-methanesulfonyl-ethylamine; hydrochloride salt (0.072 g, 0.450 mmol) is added to it, followed by the addition of TEA. The mixture is placed in a microwave and heated to 100° C. for 90 minutes. The solid in the solution is removed and the remaining solution is condensed in vacuo and is purified (preparative TLC, 5% MeOH in dichloromethane), to provide the desired product (0.004 g, 6.9%). LCMS: 438.29 (M+H⁺).

### EXAMPLE 66

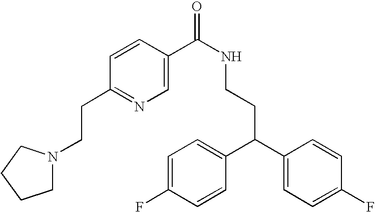

### N-[3,3-Bis-(4-fluoro-phenyl)-propyl]-6-(2-pyrrolidin-1-yl-ethyl)-nicotinamide

The title compound is prepared using the procedure from Example 1, starting from 6-(2-pyrrolidin-1-yl-ethyl)-nicotinic acid (0.089 g, 0.404 mmol) and 3,3-bis-(4-fluoro-phenyl)-propylamine (the product of Example 58, Step A) (0.100 g, 0.404 mmol). The crude product is purified (preparative TLC, 5% MeOH in dichloromethane), to provide the desired product (0.018 g, 9.9%). LCMS: 450.13 (M+H⁺).

### EXAMPLE 67

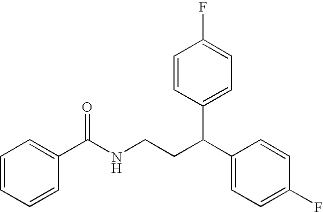

### N-[3,3-Bis-(4-fluoro-phenyl)-propyl]-benzamide

To a solution of HOBT (0.055 g, 0.407 mmol), EDC (0.08 g, 0.420 mmol) and nicotinic acid (0.025 g, 0.203 mmol) is added the solution of 3,3-bis-(4-fluoro-phenyl)-propylamine (0.050 g, 0.202 mmol) in DMF (1 mL) followed by the addition of Hünig's base (200 μL). The mixture is stirred overnight at room temperature for 15 hours. The reaction is poured into water and ether. The ether layer is washed three times with water (3×20 mL). The organic layers are combined, dried over magnesium sulfate and dried in vacuo. The resulting oil is purified by (preparative TLC, 5% 7N NH₃ in MeOH in dichloromethane), to give the desired product (0.05 μg, 71.8%). LCMS: 352.37 (M+H⁺).

### EXAMPLE 68

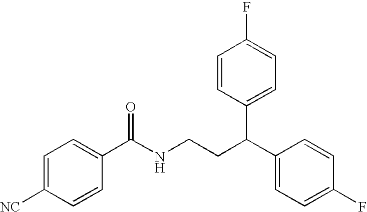

### N-[3,3-Bis-(4-fluoro-phenyl)-propyl]-4-cyano-benzamide

The title compound is prepared and purified (preparative TLC, 5% 7N NH₃ in MeOH in dichloromethane) using the procedure from Example 67, starting from 4-cyano-benzoic acid (0.029 g, 0.203 mmol) and 3,3-bis-(4-fluoro-phenyl)-propylamine (0.050 g, 0.202 mmol), to provide the desired product (0.075 g, 98.6%). LCMS: 377.3 (M+H⁺).

### EXAMPLE 69

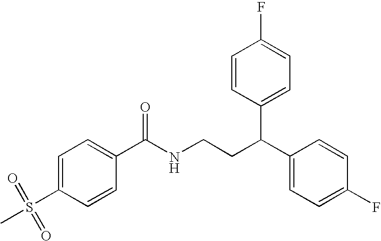

### N-[3,3-Bis-(4-fluoro-phenyl)-propyl]-4-methanesulfonyl-benzamide

The title compound is prepared and purified (preparative TLC, 5% 7N NH₃ in MeOH in dichloromethane) using the procedure from Example 67, starting from 4-methanesulfonyl-benzoic acid (0.040 g, 0.203 mmol) and 3,3-bis-(4-fluoro-phenyl)-propylamine (0.050 g, 0.202 mmol), to provide the desired product (0.073 g, 84.1%). LCMS: 430.23 (M+H⁺).

### EXAMPLE 70

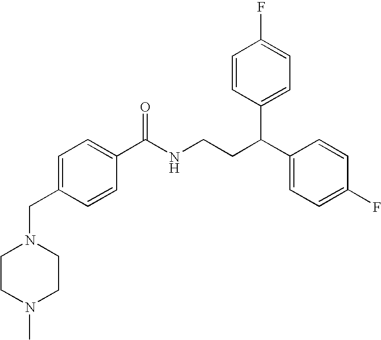

### N-[3,3-Bis-(4-fluoro-phenyl)-propyl]-4-(4-methyl-piperazin-1-ylmethyl)-benzamide

The title compound is prepared and purified (preparative TLC, 5% 7N NH₃ in MeOH in dichloromethane) using the procedure from Example 67, starting from 4-(4-methyl-piperazine-1-ylmethyl)-benzoic acid (0.047 g, 0.203 mmol) and 3,3-bis-(4-fluoro-phenyl)-propylamine (0.050 g, 0.202 mmol), to provide the desired product (0.017 g, 18.2%). LCMS: 464.34 (M+H⁺).

### EXAMPLE 71

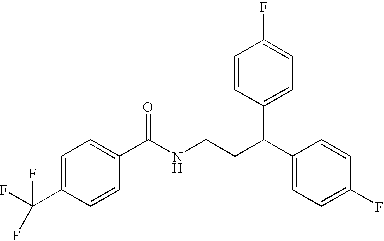

### N-[3,3-Bis-(4-fluoro-phenyl)-propyl]-4-trifluoromethoxy-benzamide

The title compound is prepared and purified (preparative TLC, 5% 7N NH₃ in MeOH in dichloromethane) using the procedure from Example 67, starting from 4-(2,2,2-trifluoro-methoxy)-benzoic acid (0.042 g, 0.204 mmol) and 3,3-bis-(4-fluoro-phenyl-propylamine (0.050 g, 0.202 mmol), to provide the desired product (0.068 g, 77.0%). LCMS: 436.24 (M+H⁺).

### EXAMPLE 72

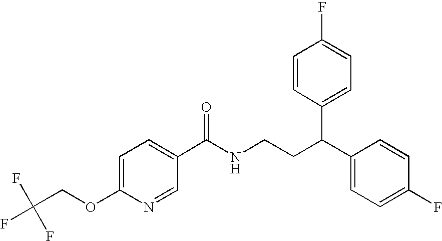

### N-[3,3-Bis-(4-fluoro-phenyl)-propyl]-6-(2,2,2-trifluoro-ethoxy)-nicotinamide

The title compound is prepared and purified (preparative TLC, 5% 7N NH₃ in MeOH in dichloromethane) using the procedure from Example 67, starting from 4-(2,2,2-trifluoro-ethoxy)-benzoic acid (0.042 g, 0.204 mmol) and 3,3-bis-(4-fluoro-phenyl)-propylamine (0.050 g, 0.202 mmol), to provide the desired product (0.068 g, 77.0%). LCMS: 451.25 (M+H⁺).

### EXAMPLE 73

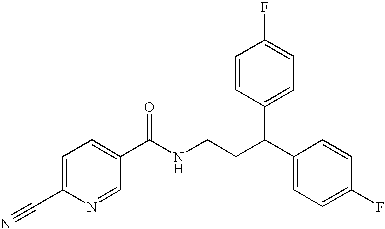

### N-[3,3-Bis-(4-fluoro-phenyl)-propyl]-6-cyano-nicotinamide

The title compound is prepared and purified (preparative TLC, 5% 7N NH₃ in MeOH in dichloromethane) using the procedure from Example 67, starting from 4-cyano-nicotinic acid (0.030 g, 0.202 mmol) and 3,3-bis-(4-fluoro-phenyl)-propylamine (0.050 g, 0.202 mmol), to provide the desired product (0.064 g, 84.0%). LCMS: 419.33 (M+H⁺).

### EXAMPLE 74

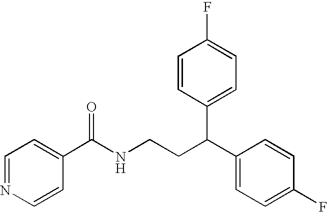

### N-[3,3-Bis-(4-fluoro-phenyl)-propyl]-isonicotinamide

The title compound is prepared and purified (preparative TLC, 5% 7N NH₃ in MeOH in dichloromethane) using the procedure from Example 67, starting from isonicotinic acid (0.025 g, 0.202 mmol) and 3,3-bis-(4-fluoro-phenyl)-propylamine (0.050 g, 0.202 mmol), to provide the desired product (0.042 g, 57.6%). LCMS: 353.36 (M+H⁺).

### EXAMPLE 75

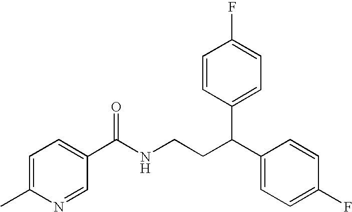

### N-[3,3-Bis-(4-fluoro-phenyl)-propyl]-6-methyl-nicotinamide

The title compound is prepared and purified (preparative TLC, 5% 7N NH₃ in MeOH in dichloromethane) using the procedure from Example 67, starting from 4-methylnicotinic acid (0.028 g, 0.202 mmol) and 3,3-bis-(4-fluoro-phenyl)-propylamine (0.050 g, 0.202 mmol), to provide the desired product (0.044 g, 59.4%). LCMS: 367.40 (M+H⁺).

### EXAMPLE 76

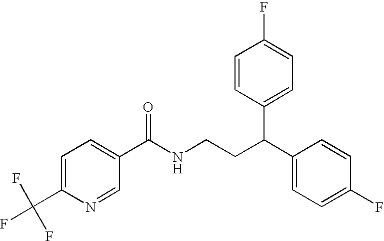

### N-[3,3-Bis-(4-fluoro-phenyl)-propyl]-6-trifluoromethyl-nicotinamide

The title compound is prepared and purified (preparative TLC, 5% 7N NH₃ in MeOH in dichloromethane) using the procedure from Example 67, starting from isonicotinic acid (0.038 g, 0.202 mmol) and 3,3-bis-(4-fluoro-phenyl)-propylamine (0.050 g, 0.202 mmol), to provide the desired product (0.059 g, 69.5%). LCMS: 462.32 (M+H⁺).

### EXAMPLE 77

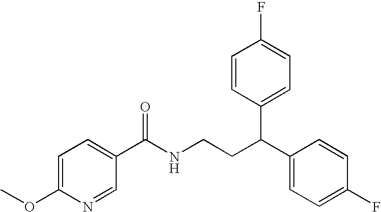

### N-[3,3-Bis-(4-fluoro-phenyl)-propyl]-6-methoxy-nicotinamide

### Step A: N-[3,3-Bis-(4-fluoro-phenyl)-propyl]-6-fluoro-nicotinamide

The title compound is prepared and purified (preparative TLC, 10% 7N NH₃ in MeOH in dichloromethane) using the procedure from Example 67, starting from 6-fluoro nicotinic acid (0.285 g, 2.022 mmol) and 3,3-bis-(4-fluoro-phenyl)-propylamine (0.500 g, 2.022 mmol) to provide the desired product (0.487 g, 65.0%).

### Step B: N-[3,3-Bis-(4-fluoro-phenyl)-propyl]-6-methoxy-nicotinamide

To the product from step A (0.055 g, 0.146 mmol) in round bottom flask is added NaOMe in methanol (2.5%, 1.5 mL, 0.303 mmol) and the mixture is heated at reflux for 2 hours and then cooled to room temperature. The reaction is quenched by the addition of water, and then the methanol is removed in vacuo. The product is extracted with dichloromethane (3×10 mL) and the combined organic layers are dried and evaporated in vacuo. The resulting yellow oil is purified (preparative TLC, 5% saturated NH₃ in MeOH/dichloromethane), to provide the desired product (0.024 g, 42.0%). LCMS: 383.34 (M+H⁺).

### EXAMPLE 78

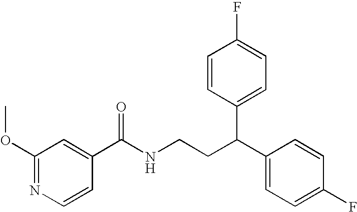

### N-[3,3-Bis-(4-fluoro-phenyl)-propyl]-2-methoxy-isonicotinamide

### Step A: N-[3,3-Bis-(4-fluoro-phenyl)-propyl]-2-fluoro-isonicotinamide

The coupled product is prepared and purified (preparative TLC, 10% 7N NH₃ in MeOH in dichloromethane) using the procedure from Example 67, starting from 1-fluoro isonicotinic acid (0.571 g, 4.050 mmol) and 3,3-bis-(4-fluoro-phenyl)-propylamine (1.000 g, 4.044 mmol) to provide the desired compound (1.110 g, 74.1%).

### Step B: N-[3,3-Bis-(4-fluoro-phenyl)-propyl]-2-methoxy-isonicotinamide

The title compound is prepared and purified (preparative TLC, 5% 7N NH₃ in MeOH/dichloromethane) using the same procedure in Example 77, Step B, starting from the product of Step A (0.038 g, 0.202 mmol), to provide the desired product (0.041 g, 73.0%). LCMS: found: 383.34 (M+H⁺).

### EXAMPLE 79

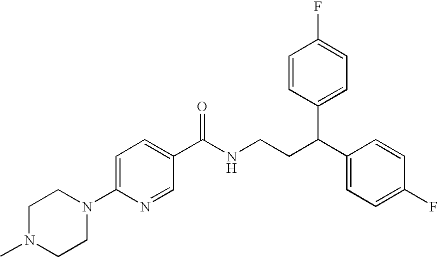

### N-[3,3-Bis-(4-fluoro-phenyl)-propyl]-6-(4-methyl-piperazin-1-yl)-nicotinamide

To the solution of the product from Example 77, Step A (0.060 g, 0.162 mmol) in THF (2.5 mL), is added 1-methyl piperazine (0.045 g, 0.450 mmol) and the mixture is heated in microwave at 140° C. for 3 hours.

The solvent is removed in vacuo and the crude oil is purified (preparative TLC, 5% 7N NH₃ in MeOH in dichloromethane) to provide the desired product (0.017 g, 23.3%). LCMS: 451.40 (M+H⁺).

### EXAMPLE 80

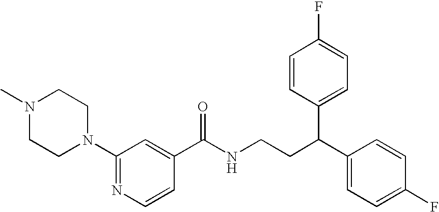

### N-[3,3-Bis-(4-fluoro-phenyl)-propyl]-2-(4-methyl-piperazin-1-yl)-isonicotinamide

The title compound is prepared and purified (preparative TLC, 5% 7N NH₃ in MeOH in dichloromethane) using the procedure from Example 79, starting from the product of Example 78, Step A (0.065 g, 0.175 mmol) and 1-methyl piperazine (0.050 g, 0.500 mmol, to provide the desired product (0.018 g, 22.8%). LCMS: 451.42 (M+H⁺).

### EXAMPLE 81

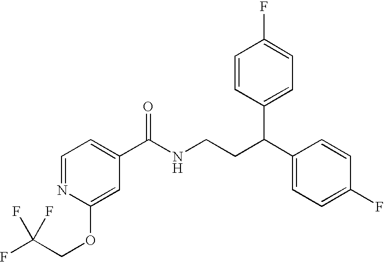

### N-[3,3-Bis-(4-fluoro-phenyl)-propyl]-2-(2,2,2-trifluoro-ethoxy)-isonicotinamide

The title compound is prepared and purified (preparative TLC, 5% saturated NH₃ in MeOH in dichloromethane) using the procedure from Example 79, starting from the product of Example 78, Step A (0.150 g, 0.405 mmol) and 2,2,2-trifluoroethanol (0.300 mL, 0.500 mmol) and NaH (0.016 g, 0.405 mmol), to provide the desired product (0.061 g, 33.4%). LCMS: 492.31 (M+H⁺).

### EXAMPLE 82

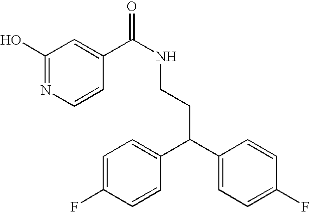

### N-[3,3-Bis-(4-fluoro-phenyl)-propyl]-2-hydroxy-isonicotinamide

The title compound is prepared and purified (flash chromatography, 5% MeOH in dichloromethane) using the procedure from Example 32, starting from 2-hydroxy isonicotinic acid (0.024 g, 0.178 mmol) and 3,3-bis-(4-fluoro-phenyl)-propylamine (0.044 g, 0.178 mmol), to provide the desired product (0.012 g, 18.3%). LCMS: 369.37 (M+H⁺).

### EXAMPLE 83

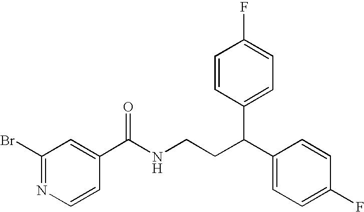

### N-[3,3-Bis-(4-fluoro-phenyl)-propyl]-2-bromo-isonicotinamide

The title compound is prepared and purified (flash chromatography, MeOH in dichloromethane, then crystallization from ether and hexanes) using the procedure from Example 67, starting from 2-bromo isonicotinic acid (0.081 g, 0.404 mmol) and 3,3-bis-(4-fluoro-phenyl)-propylamine (0.100 g, 0.404 mmol), to provide the desired product (0.078 g, 44.7%). LCMS: 474.29 (M+H⁺).

### EXAMPLE 84

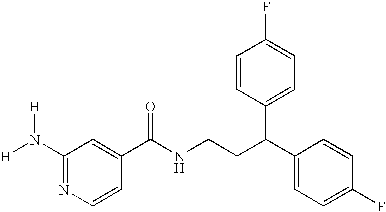

### 2-Amino-N-[3,3-bis-(4-fluoro-phenyl)-propyl]-isonicotinamide

The title compound is prepared and purified (preparative TLC 10%, saturated NH₃ in MeOH in dichloromethane) using the procedure from Example 67, starting from 2-amino isonicotinic acid (0.028 g, 0.202 mmol) and 3,3-bis-(4-fluoro-phenyl)-propylamine (0.050 g, 0.202 mmol), to provide the desired product (0.018 g, 24.2%). LCMS: 368.37 (M+H⁺).

### EXAMPLE 85

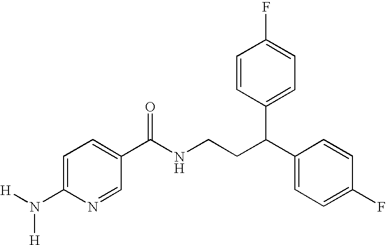

### 6-Amino-N-[3,3-bis-(4-fluoro-phenyl)-propyl]-nicotinamide

The title compound is prepared and purified (preparative TLC, 10% saturated NH₃ in MeOH in dichloromethane) using the procedure from Example 67, starting from 6-amino nicotinic acid (0.028 g, 0.202 mmol) and 3,3-bis-(4-fluoro-phenyl)-propylamine (0.050 g, 0.202 mmol), to provide the desired product (0.013 g, 17.5%). LCMS: 368.39 (M+H⁺).

### EXAMPLE 86

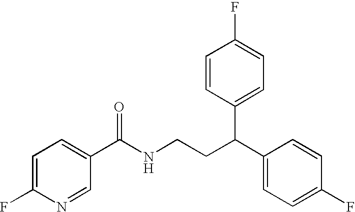

### N-[3,3-Bis-(4-fluoro-phenyl)-propyl]-6-fluoro-nicotinamide

The title compound is made in Example 77, Step A. LCMS: found: (M+H⁺), 371.372.

### EXAMPLE 87

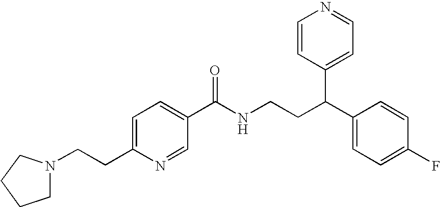

### N-[3-(4-Fluoro-phenyl)-3-pyridin-4-yl-propyl]-6-(2-pyrrolidin-1-yl-ethyl)-nicotinamide

### Step A: (E)-3-(4-Fluoro-phenyl)-3-pyridin-4-yl-acrylonitrile

To acetonitrile is added crushed potassium hydroxide (4.179 g, 74.64 mmol) and the mixture is stirred for 5 minutes. (4-(4-Fluorobenzoyl)pyridine is added to this, and the mixture turns red immediately. The mixture is stirred overnight at room temperature. To the mixture is added water and ether and the organic phase is condensed in vacuo to provide the desired, crude product (0.450 g, 71.8%).

### Step B: (Z)-3-(4-Fluoro-phenyl)-3-pyridin-4-yl-allylamine

To a nitrogen filled flask is added 10% palladium on carbon (wet) followed by the addition of ethanol. The product from Step A is added to that followed by the addition of more ethanol. To the flask is attached a balloon of H₂ and the reaction is stirred at room temperature overnight until completion of the reaction. The crude reaction product was immediately subjected to the reaction conditions described below in step C.

### Step C: 3-(4-Fluoro-phenyl)-3-pyridin-4-yl-propylamine

To the product from Step B which contains palladium on carbon is added 50 mL ethanol and the mixture is hydrogenated at room temperature, at 50 psi, overnight. The reaction mixture is filtered through diatomaceous earth and the resulting filtrate is evaporated in vacuo and purified (flash chromatography, MeOH in dichloromethane) to provide the desired product (87% combined yiled for two steps, B and C).

### Step D: N-[3-(4-Fluoro-phenyl)-3-pyridin-4-yl-propyl]-6-(2-pyrrolidin-1-yl-ethyl)-nicotinamide

The title compound is prepared and purified (preparative TLC, 10% 7N NH₃ in MeOH in dichloromethane) using the procedure from Example 67, starting from 6-(pyrrolidin-1-yl-ethyl)-nicotinic acid (0.050 g, 0.227 mmol) and product from Step C (0.052 g, 0.227 mmol) to provide the desired product (0.031 g, 31.6%). LCMS: 433.55 (M+H⁺).

### EXAMPLE 88

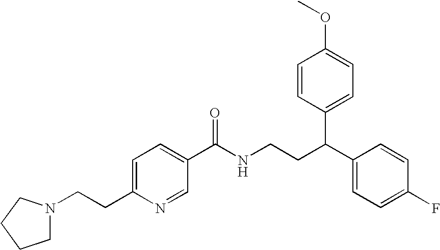

### N-[3-(4-Fluoro-phenyl)-3-(4-methoxy-phenyl)-propyl]-6-(2-pyrrolidin-1-yl-ethyl)-nicotinamide

### Step A: (Z)-3-(4-Fluoro-phenyl)-3-(4-methoxy-phenyl)-acrylonitrile

Sodium hydride (0.088 g, 2.200 mmol) is washed three times with hexanes and dried under vacuum. It is then suspended in THF (15 mL) and cooled to 0° C. in an ice bath. (2-Cyano-ethyl)-phosphonic acid diethyl ester (0.389 g, 2.200 mmol) in 10 mL THF, is added dropwise, to the reaction over 5 min. The reaction is warmed to room temperature and stirred for 20 minutes and cooled again in an ice bath to 0° C. (4-Fluoro-phenyl)-(4-methoxy-phenyl)-methanone (0.5 g, 2.172 mmol) is added (in 10 mL of THF) dropwise to the reaction mixture over the course of 10 minutes and the reaction is stirred, at room temperature, for 3 days. It is then cooled to 0° C. and quenched by the dropwise addition of water. The reaction mixture is neutralized by the addition of saturated NH₄Cl and extracted twice with 50% ether in petroleum ether. The organic layers are dried over magnesium sulfate, filtered, evaporated in vacuo and purified (flash chromatography, MeOH in dichloromethane) to give the desired product (0.175 g, 31.8%).

### Step B: 3-(4-Fluoro-phenyl)-3-pyridin-4-yl-propylamine

The product of step A and palladium on carbon in ethanol (50 mL) are placed into a Parr bomb and hydrogenated at room temperature, at 50 psi, overnight. The reaction is filtered through diatomaceous earth and the solvents are evaporated in vacuo and purified (flash chromatography, MeOH in dichloromethane) to give the desired product (260 mg, 63%)

### Step C: N-[3-(4-Fluoro-phenyl)-3-(4-methoxy-phenyl)-propyl]-6-(2-pyrrolidin-1-yl-ethyl)-nicotinamide

The title compound is prepared and purified (preparative TLC, 10% 7N NH₃ in MeOH in dichloromethane) using the procedure from Example 67, starting from 6-(pyrrolidin-1-yl-ethyl)-nicotinic acid (0.050 g, 0.227 mmol) and the product from Step B (0.058 g, 0.227 mmol), to provide the desired product (0.043 g, 41.0%). LCMS: 462.46 (M+H⁺).

### EXAMPLE 89

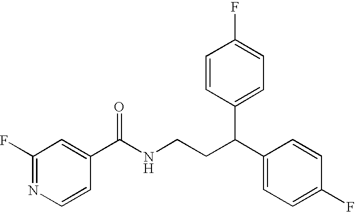

### N-[3,3-Bis-(4-fluoro-phenyl)-propyl]-2-fluoro-isonicotinamide

The title compound is made in Example 78, Step A. LCMS: 371.37 (M+H⁺).

### EXAMPLE 90

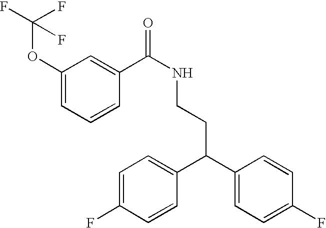

### N-[3,3-Bis-(4-fluoro-phenyl)-propyl]-3-trifluoromethoxy-benzamide

The title compound is prepared and purified (preparative TLC, 30% EtOAc in hexanes) using the procedure from Example 32, starting from 3-trifluoromethoxy-benzoic acid (0.100 g, 0.485 mmol) and 3,3-bis-(4-fluoro-phenyl)-propylamine (0.120 g, 0.485 mmol), to provide the desired product (0.062 g, 29.4%). LCMS: 436.31 (M+H⁺).

### EXAMPLE 91

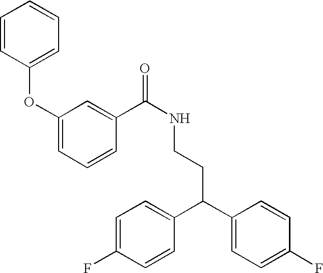

### N-[3,3-Bis-(4-fluoro-phenyl)-propyl]-3-phenoxy-benzamide

The title compound is prepared and purified (flash chromatography, 30% EtOAc in hexanes) using the

procedure from Example 32, starting from 3-phenoxy-benzoic acid (0.104 g, 0.485 mmol) and 3,3-bis-(4-fluoro-phenyl)-propylamine (0.120 g, 0.485 mmol), to provide the desired product (0.071 g, 33.3%). LCMS: 444.35 (M+H⁺).

### EXAMPLE 92

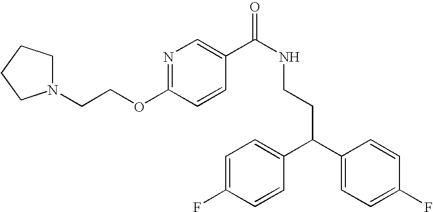

### N-[3,3-Bis-(4-fluoro-phenyl)-propyl]-6-(2-pyrrolidin-1-yl-ethoxy)-nicotinamide

To a solution of product from Example 77, Step A, in DMF (0.050 g, 0.135 mmol) is added 2-pyrrolidine-1-yl-ethanol (0.016 g, 0.135 mmol) followed by the addition of sodium hydride (60% emulsion, 0.006 g, 0.162 mmol). The mixture is placed in a microwave for 20 minutes at 120° C. To the mixture is added ether and the ether layer is wahed with water, and evaporated in vacuo, to provide the desired product (0.056 g, 89.1%). LCMS: 466.41 (M+H⁺).

### EXAMPLE 93

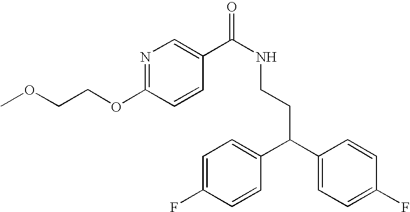

### N-[3,3-Bis-(4-fluoro-phenyl)-propyl]-6-(2-methoxy-ethoxy)-nicotinamide

The title compound is prepared and purified using the procedure from Example 92, using 2-methoxy-ethanol (0.051 mL, 0.675 mmol) and the product of Example 77, Step A (0.050 g, 0.135 mmol) to provide the desired product (0.057 g, 99.0%). LCMS: 427.35 (M+H⁺).

### EXAMPLE 94

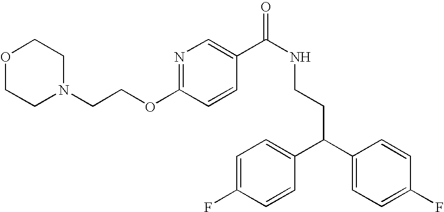

### N-[3,3-Bis-(4-fluoro-phenyl)-propyl]-6-(2-morpholin-4-yl-ethoxy)-nicotinamide

The title compound is prepared and purified using the procedure from Example 92, using 2-morpholin-4-yl-ethanol (0.088 g, 0.0.675 mmol) and the product of Example 77, Step A (0.050 g, 0.135 mmol), to provide the desired product (0.061 g, 93.8%). LCMS: 482.36 (M+H⁺).

### EXAMPLE 95

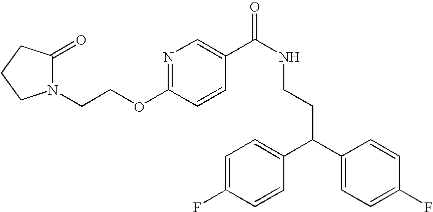

### N-[3,3-Bis-(4-fluoro-phenyl)-propyl]-6-[2-(2-oxo-pyrrolidin-1-yl)-ethoxy]-nicotinamide

The title compound is prepared and purified using the procedure from Example 92, using 1-(2-hydroxy-ethyl)-pyrrolidine-2-one (0.088 g, 0.0.675 mmol) and the product of Example 77, Step A (0.050 g, 0.135 mmol) to provide desired product (0.061 g, 93.8%). LCMS: 480.39 (M+H⁺).

### EXAMPLE 96

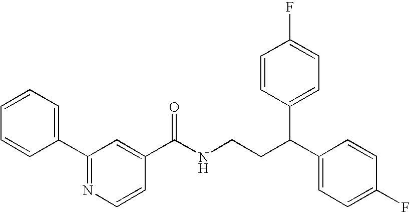

### N-[3,3-Bis-(4-fluoro-phenyl)-propyl]-2-phenyl-isonicotinamide

The product of Example 83 (0.043 g, 0.093 mmol) is dissolved in THF and water (1:0.3 mL) followed by the addition of phenyl boronic acid (0.0182, 0.150 mmol), palladium tetrakistriphenylphosphine (0.008 g, 0.007 mmol) and cesium carbonate (0.090 g, 0.227 mmol). The reaction vessel is sealed, filled with nitrogen and heated to 60° C. for 4 hours. The reaction mixture is then poured into water, extracted with ethyl acetate, evaporated in vacuo and purified (preparative TLC, MeOH in dichloromethane) to provide the desired product (0.018 g, 25.3%). LCMS: 429.33 (M+H⁺).

### EXAMPLE 97

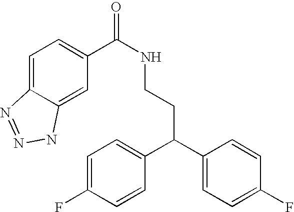

### 3H-Benzotriazole-5-carboxylic acid [3,3-bis-(4-fluoro-phenyl)-propyl]-amide

The title compound is prepared using the procedure from Example 1, starting from benzotriazole-5-carboxylic acid (0.100 g, 0.613 mmol) and the product of Example 58, Step A (0.189, 0.613 mmol). The resulting compound is purified (preparative TLC, 5% MeOH in dichloromethane), to provide the desired product (0.040 g, 16.6%). LCMS: 393.29 (M+H⁺).

### EXAMPLE 98

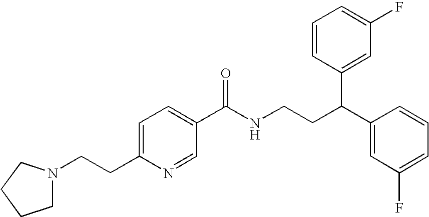

### N-[3,3-Bis-(3-fluoro-phenyl)-propyl]-6-(2-pyrrolidin-1-yl-ethyl)-nicotinamide

### Step A: 3,3-Bis-(3-fluoro-phenyl)-acrylonitrile

This compounds is prepared according to the procedure in Example 88, Step A.

### Step B: 3,3-Bis-(3-fluoro-phenyl)-propylamine

This compound is prepared (with addition of 3 mL of acetic acid into the reaction mixture before hydrogenation) and purified (preparative TLC, MeOH in dichloromethane) using the method from Example 88, Step B, starting from the product of Step A (0.600 g, 2.487 mmol) to give the desired product (0.245 g, 39.8%).

### Step C: N-[3,3-Bis-(3-fluoro-phenyl)-propyl]-6-(2-pyrrolidin-1-yl-ethyl)-nicotinamide

The title compound is prepared and purified (preparative TLC, 10% saturated NH₃ in MeOH in dichloromethane) using the procedure from Example 67, starting from 6-(pyrrolidin-1-yl-ethyl)-nicotinic acid (0.046 g, 0.209 mmol) and the product from Step B (0.050 g, 0.202 mmol), to provide the desired product (0.044 g, 48.4%). LCMS: 450.37 (M+H⁺).

### EXAMPLE 99

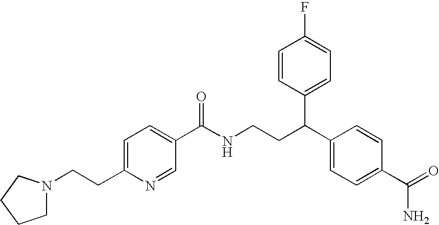

### N-[3-(4-Carbamoyl-phenyl)-3-(4-fluoro-phenyl)-propyl]-6-(2-pyrrolidin-1-yl-ethyl)-nicotinamide

### Step A: 4-[(E)-2-Cyano-1-(4-fluoro-phenyl)-vinyl]-benzamide

(Z)-3-Chloro-3-(4-fluoro-phenyl)-acrylonitrile (0.0170, 0.936 mmol) (0.164 g, 1.516 mmol) is dissolved in THF and water (2:1 mL) followed by the addition of 4-acetamido boronic acid, palladium acetate (0.055 g, 0.047 mmol), tetrabutylammonium bromide (0.222 g, 0.689 mmol) and potassium carbonate (0.276 g, 2.000 mmol). The reaction vessel is sealed, filled with nitrogen and heated to 60° C. for 4 hours. The reaction is then poured into water, extracted with ethyl acetate, and purified (flash chromatography, MeOH in dichloromethane) to give the desired product (0.088 g, 35.3%).

### Step B: 4-[3-Amino-1-(4-fluoro-phenyl)-propyl]-benzamide

This compound is prepared (with addition of 3 mL of acetic acid to the reaction mixture before hydrogenation) and purified (flash chromatography, MeOH in dichloromethane) using the method from Example 88 Step B, starting from the product of Step A (0.320 g, 1.202 mmol), to provide the desired product (0.057 g, 17.4%).

### Step C: N-[3-(4-Carbamoyl-phenyl)-3-(4-fluoro-phenyl)-propyl]-6-(2-pyrrolidin-1-yl-ethyl)-nicotinamide

The title compound is prepared and purified (preparative TLC, 10% saturated NH₃ in MeOH in dichloromethane) using the procedure from Example 67, starting from 6-(pyrrolidin-1-yl-ethyl)-nicotinic acid (0.020 g, 0.091 mmol) and product from step B (0.021 g, 0.077 mmol), to provide the desired product (0.030 g, 82.0%). LCMS: 475.38 (M+H⁺).

### EXAMPLE 100

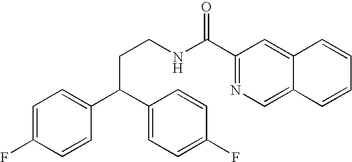

### Isoguinoline-3-carboxylic acid [3,3-bis-(4-fluoro-phenyl)-propyl]-amide

To a solution of isoquinoline-3-carboxylic acid (0.200, g, 1.155 mmol) in anhydrous toluene (3 mL) cooled to 0° C., is added 253 mL of thionyl chloride, and the mixture is stirred for 20 minutes. The temperature is increased to 50° C. and the mixture is stirred for additional 30 minutes until the formation of white solid in the mixture. It is then cooled and the solvents are removed in vacuo, followed by the addition of toluene (2×3 mL) and their removal in vacuo. Fresh toluene (3 mL) is then added to the mixture and it is cooled to 0° C. To the mixture is added Hünig's base (502 μL, 2.89 mmol) followed by the addition of amine (0.343 g, 1.39 mmol) and stirred for half an hour. The reaction mixture is allowed to warm to room temperature and is stirred for another 2 hours. To the reaction is added 10 mL of water followed by the addition of HCl (4 mL, 1 N). The layers are separated and the aqueous layer is washed with toluene and the combined organic fractions are washed with water (10 mL), brine (10 mL), and dried over sodium sulfate. Toluene is decanted and filtered through silica gel using toluene (40 mL). The resulting solution is evaporated and dissolved in (dichloromethane, 8 mL), washed with saturated sodium carbonate (15 mL), dried by passing through silica plug, and eluting with dichloromethane. The solvents are removed in vacuo, to provide desired product (0.290 g, 62.5%). LCMS: 403.00 (M+H⁺).

### EXAMPLE 101

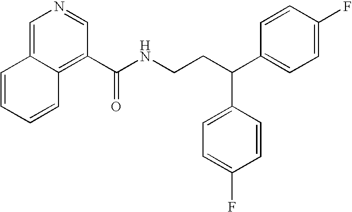

### Isoguinoline-4-carboxylic acid [3,3-bis-(4-fluoro-phenyl)-propyl]-amide

### Step A: Isoguinoline-4-carbonitrile

To 4-bromo isoquinoline (2 g, 9.613 mmol) is added copper (I) cyanide (1.29 g, 14.42 mmol), and the mixture is heated to 250° C. for 45 minutes, where the pressure is taken down to 5-10 torr. The mixture which turns black at this point begins to distil over, giving crystals in the condenser. The condenser is cleaned with dichloromethane and the volatiles in the solution are removed in vacuo to give the desired product (0.66 g, 44.6%) as colorless crystals.

### Step B: Isoquinoline-4-carboxylic acid hydrochloride

The product from step A (0.66 g, 4.2 mmol) is dissolved in concentrated HCl (6 ml) and heated in a sealed tube for 7 hours. It is then cooled and water is removed in vacuo to the desired product as a white powder.

### Step C: Isoguinoline-4-carboxylic acid [3,3-bis-(4-fluoro-phenyl)-propyl]-amide

To a solution of the product from Step B (0.2 g, 1.155 mmol) in toluene (6 mL) under argon, is added DMF (1 mL), followed by thionyl chloride (146 μL). Reaction is allowed to stand for 30 minutes at room temperature, heated to 80° C. for one hour and then cooled. The volatiles are removed in vacuo and fresh toluene (2×6 mL) is added to the mixture and the solvents are removed in vacuo. Toluene (6 ml) is added to the mixture and cooled to −10° C., followed by the addition of Hünig's base (502 μL) and 3,3-bis-(4-fluoro-phenyl)-propyl]-amine (0.343 g, 1.386 mmol). The mixture is stirred at −10° C. for 30 minutes and then allowed to warm to room temperature and stirred overnight. The volatiles are removed in vacuo and the resulting compound is purified on silica gel (EtOAc/heptane). The fraction containing the product is collected and evaporated in vacuo to give the desired product (0.083, 18.2%). LCMS: 403.00 (M+H⁺).

### EXAMPLE 102

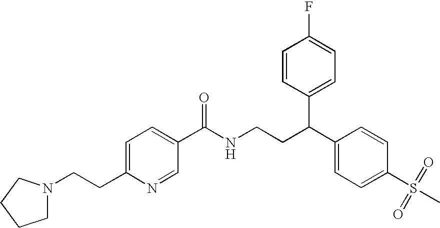

### N-[3-(4-Fluoro-phenyl)-3-(4-methanesulfonyl-phenyl)-propyl]-6-(2-pyrrolidin-1-yl-ethyl)-nicotinamide

### Step A: (E)-3-(4-Fluoro-phenyl)-3-(4-methanesulfonyl-phenyl)-acrylonitrile

(Z)-3-Chloro-3-(4-fluoro-phenyl)-acrylonitrile (0.125 g, 0.688 mmol) is dissolved in THF (mL) followed by the addition of 4-methylsulfamido boronic acid (0.150 g, 0.750 mmol), palladium acetate (0.015 g, 0.067 mmol) and potassium carbonate (0.290 g, 0.892 mmol). The reaction vessel is sealed, filled with nitrogen and heated to 85° C. for 14 hours to give the desired product (100 mg, 50.0%)

### Step B: 3-(4-Fluoro-phenyl)-3-(4-methanesulfonyl-phenyl)-propylamine

This compound is prepared (with addition of 3 mL of acetic acid to the reaction mixture before hydrogenation) and purified (flash chromatography, MeOH in dichloromethane) using the method from Example 88 Step B, starting from the product of Step A (0.150 g, 0.498 mmol) to provide the desired product (0.067 g, 43.79%).

### Step C: N-[3-(4-Fluoro-phenyl)-3-(4-methanesulfonyl-phenyl)-propyl]-6-(2-pyrrolidin-1-yl-ethyl)-nicotinamide

The title compound is prepared and purified (preparative TLC, 10% sat NH₃ in MeOH in dichloromethane) using the procedure from Example 67, starting from 6-(pyrrolidin-1-yl-ethyl)-nicotinic acid (0.048 g, 0.218 mmol) and the product of Example 102, Step B (0.070 g, 0.228 mmol) to provide the desired product (0.024 g, 20.7%). LCMS: 510.28 (M+H⁺).

### EXAMPLE 103

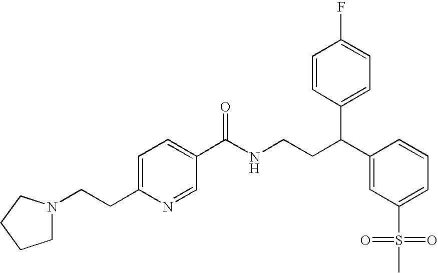

### N-[3-(4-Fluoro-phenyl)-3-(3-methanesulfonyl-phenyl)-propyl]-6-(2-pyrrolidin-1-yl-ethyl)-nicotinamide

### Step A: (E)-3-(4-Fluoro-phenyl)-3-(3-methanesulfonyl-phenyl)-acrylonitrile.

(Z)-3-Chloro-3-(4-fluoro-phenyl)-acrylonitrile (0.400 g, 2.203 mmol) is dissolved in THF (3.6 mL) followed by the addition of 3-methylsulfamido boronic acid (0.460 g, 2.300 mmol), palladium tris dibenzylidine acetone (0.020 g, 0.022 mmol), potassium tri tert-butyl phosphine tetrafluoroborate (0.013 g, 0.045 mmol) and potassium fluoride (0.415 g, 7.143 mmol). The reaction vessel is sealed, filled with nitrogen and heated to 40° C. overnight to provide the desired product. (736 mg, 90%)

### Step B: 3-(4-Fluoro-phenyl)-3-(3-methanesulfonyl-phenyl)-propylamine

This compounds is prepared (with addition of 3 mL of acetic acid to the reaction mixture before hydrogenation) and purified (flash chromatography, MeOH in dichloromethane) using the method from Example 88 Step B, starting from the product of Step A (0.450 g, 1.493 mmol) to provide the desired product (0.321 g, 69.9%).

### Step C: N-[3-(4-Fluoro-phenyl)-3-(3-methanesulfonyl-phenyl)-propyl]-6-(2-pyrrolidin-1-yl-ethyl)-nicotinamide

The title compound is prepared and purified (preparative TLC, 10% sat NH₃ in MeOH in dichloromethane) using the procedure from Example 67, starting from 6-(pyrrolidin-1-yl-ethyl)-nicotinic acid (0.048 g, 0.218 mmol) and the product of Step B (0.070 g, 0.228 mmol) to provide the desired product (0.024 g, 20.7%). LCMS: 510.28 (M+H⁺).

### EXAMPLE 104

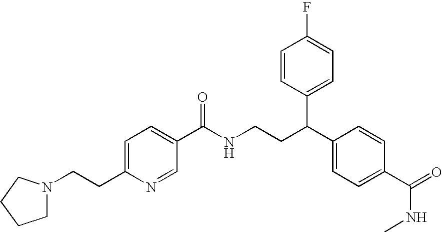

### N-[3-(4-Fluoro-phenyl)-3-(4-methylcarbamoyl-phenyl)-propyl]-6-(2-pyrrolidin-1-yl-ethyl)-nicotinamide

### Step A: 4-[(Z)-2-Cyano-1-(4-fluoro-phenyl)-vinyl]-N-methyl-benzamide

(Z)-3-Chloro-3-(4-fluoro-phenyl)-acrylonitrile (3.700 g, 20.38 mmol) is dissolved in THF (35 mL) followed by the addition of 4-methyl amido boronic acid (4.09 g, 22.85 mmol), palladium tris dibenzylidine acetone (0.940 g, 1.027 mmol), potassium tri t-butyl phosphine tetrafluoro borate (0.608 g, 2.10 mmol) and potassium fluoride (3.700 g, 63.68 mmol). The reaction vessel is sealed, filled with nitrogen and heated to 45° C. for 18 hours. It is then cooled to room temperature and passed through a pad of silica gel, and the resulting solution is evaporated in vacuo to provide the desired product. (4.800 g, 86.0%).

### Step B: 3-(4-Fluoro-phenyl)-3-(3-methanesulfonyl-phenyl)-propylamine

This compound is prepared (with addition of 3 mL of acetic acid to the reaction mixture before hydrogenation) and purified (flash chromatography, MeOH in dichloromethane) using the method from Example 88 Step B, starting from the product of Step A (0.266 g, 0.949 mmol) to provide the desired product (0.040 g, 14.7%).

### Step C: N-[3-(4-Fluoro-phenyl)-3-(4-methylcarbamoyl-phenyl)-propyl]-6-(2-pyrrolidin-1-yl-ethyl)-nicotinamide

The title compound is prepared and purified (preparative TLC, 10% sat NH₃ in MeOH in dichloromethane) using the procedure from Example 67, starting from 6-(pyrrolidin-1-yl-ethyl)-nicotinic acid (0.028 g, 0.127 mmol) and the product of Example 168, Step B (0.035 g, 0.122 mmol), to provide the desired product (0.012 g, 20.1%). LCMS: 489.33 (M+H⁺).

### EXAMPLE 105

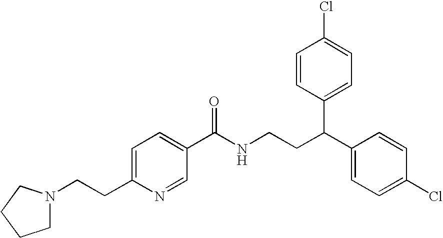

### N-[3,3-Bis-(4-chloro-phenyl)-propyl]-6-(2-pyrrolidin-1-yl-ethyl)-nicotinamide

### Step A: 3,3-Bis-(4-chloro-phenyl)-2-cyano-acrylic acid ethyl ester

To a solution of (Z)-3-(4-chloro-phenyl)-2-cyano-acrylic acid ethyl ester (36.00 g, 152.8 mmol) in anhydrous toluene (550 mL) is slowly added 4-chlorophenyl magnesium bromide (1 M in ether, 199 mL, 198.6 mmol), and the reaction mixture is stirred vigorously. The mixture is heated at reflux for 1 hour. The reaction is monitored by TLC (3:1 heptane: ethyl acetate). When the reaction is complete, the solution is poured into a mixture of 400 g of ice and HCl (4 N, 55 mL). The water layer is separated and washed with ethyl acetate (1×200 mL). The combined organic fraction is washed with sodium bicarbonate (1×200 mL), brine and dried over sodium sulfate. The resulting solution is dried in vacuo to give the desired product (56.00 g, 100%).

### Step B: 2-Carbamoyl-3,3-bis-(4-chloro-phenyl)-acrylic acid

The product from step A (150 mmol) is taken up in a flask and heated at reflux with NaOH (25 g, 600 mmol) in water (500 mL) for two hours. It is then cooled to room temperature and washed with MTBE (2×200 mL), acidified with HCl (80 mL), and washed with ethyl acetate (3×200 mL). The ethyl acetate layer is dried over sodium sulfate and stripped in vacuo to desired compound along with two other impurities.

### Step C: 3,3-Bis-(4-chloro-phenyl)-propionamide and 3,3-bis-(4-chloro-phenyl)-propionitrile

To the product from step B (21 g, 62.09 mmol) in DMSO (200 mL), is added lithium chloride (5.27 g, 124.18 mmol) and the mixture is heated to 130° C. for one hour. The solution is heated for an additional hour and cooled to room temperature. Water (250 mL) and ethyl acetate (10 mL) are added to the mixture and it is shaken vigorously. The ethyl acetate layer is drained and the water layer is washed one more time with ethyl acetate (100 mL). The combined ethyl acetate layer is, washed with water (2×100 mL), saturated sodium bicarbonate (1×100 mL), brine (1×100 mL), and dried over sodium sulfate. The volatiles are removed in vacuo, to give 3,3-bis-(4-chloro-phenyl)-propionamide and 3,3-bis-(4-chloro-phenyl)-propionitrile (2:1).

### Step D: 3,3-Bis-(4-chloro-phenyl)-propylamine

To the product from Step C (15.95 g) in THF (150 mL), is slowly added LiAlH₄ (1M, 110 mL) at −78° C., and the mixture is stirred for 8 hours and it is allowed to reach room temperature. It is stirred overnight and then cooled to 0° C. To this water is added dropwise (4 mL) followed by the addition of sodium sulfate decahydrate, until the mixture is completely quenched. The resulting salts are filtered and washed with THF (5×50 mL). The THF layer is evaporated in vacuo to give amber-colored oil (16 g). The oil is dissolved in MTBE (250 mL), washed with HCl (1×200 mL), brine (1×200 mL) and the solvents are removed in vacuo to provide a foamy yellow solid. To the solid is added ethyl acetate (40 mL) and heated to 40° C. to give a suspension. Heptane is added to the mixture and heated for 30 minutes. The mixture is then cooled to 0° C. and filtered to leave a white to off white solid. The mother liquor is evaporated and ethyl acetate (20 mL) is added to that to provide cloudy slurry which is added dropwise to heptane (250 mL). The off white precipitate formed is filtered off. The combined precipitates are ground together into a very fine powder and then re-triturated in heptane: ethyl acetate (5:1 mL) and filtered to provide off white powder. The powder is dissolved in heptane: acetone (500:200 mL), heated, cooled and filtered to provide a yellow solid (9.2 g). The solid is boiled in chloroform (100 mL), chilled to −10° C., and filtered to provide a white solid. The filtrate is concentrated, re-triturated in chloroform, and filtered to provide white solid which is combined with the other solid (7.89 g).

### Step E: N-[3,3-Bis-(4-chloro-phenyl)-propyl]-6-(2-pyrrolidin-1-yl-ethyl)-nicotinamide

The title compound is prepared and purified (flash chromatography, 10% sat NH₃ in MeOH in dichloromethane) using the procedure from Example 67, starting from 6-(pyrrolidin-1-yl-ethyl)-nicotinic acid (0.084 g, 0.381 mmol) and the product of Example 105, step A (0.150 g, 0.535 mmol), to provide the desired product (0.124 g, 48.0%). LCMS: 483.45, (M+H⁺).

### EXAMPLE 106

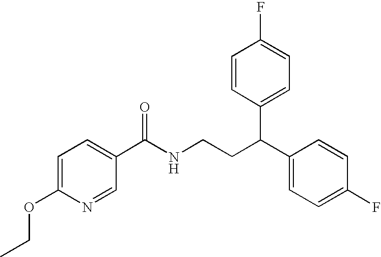

### N-[3,3-Bis-(4-fluoro-phenyl)-propyl]-6-ethoxy-nicotinamide

To ethanol (2 mol) is added NaH (0.017 g, 0.440 mmol) followed by the addition of the product of Example 77, Step A (0.148 g, 0.400 mmol), and the mixture is stirred for 10 minutes. The mixture is heated to reflux overnight. The volatiles are removed in vacuo and the resulting oil is purified using (flash chromatography, ethyl acetate), to provide the desired product (0.135 g, 85.1%). LCMS: 397.31 (M+H⁺).

### EXAMPLE 107

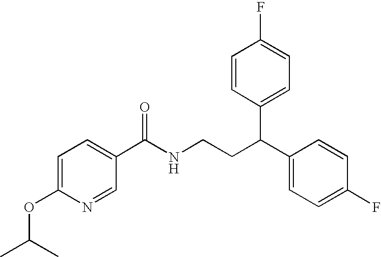

### N-[3,3-Bis-(4-fluoro-phenyl)-propyl]-6-isopropoxy-nicotinamide

The title compound is prepared and purified (flash chromatography, ethyl acetate) using the procedure from Example 106, using isopropyl alcohol (2 mL) to provide the desired product (0.079 g, 48.1%). LCMS: 411.09 (M+H⁺).

### EXAMPLE 108

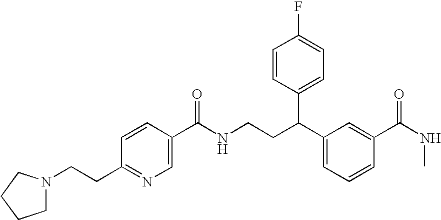

### N-[3-(4-Fluoro-phenyl)-3-(3-methylcarbamoyl-phenyl)-propyl]-6-(2-pyrrolidin-1-yl-ethyl)-nicotinamide

### Step A: 3-[(E)-2-Cyano-1-(4-fluoro-phenyl)-vinyl]-N-methyl-benzamide

(Z)-3-Chloro-3-(4-fluoro-phenyl)-acrylonitrile (0.5 g, 2.753 mmol) is dissolved in dioxane (8 mL) followed by the addition of 3-methyl amido phenyl-boronic acid (0.690 g, 3.855 mmol), palladium acetate (0.061 g, 0.272 mmol), potassium carbonate (0.728 g, 5.275 mmol). The reaction vessel is sealed, filled with nitrogen and heated to 85° C. for 14 hours. It is then cooled to room temperature and purified (flash chromatography, MeOH in dichloromethane) to provide the desired compound (0.241 g, 22.3%).

### Step B; 3-[2-Cyano-1-(4-fluoro-phenyl)-ethyl]-N-methyl-benzamide

This compound is prepared (with addition of 3 mL of acetic acid to the reaction mixture before hydrogenation) and purified MeOH in dichloromethane using the method from Example 88 Step B, starting from the product of Step A (0.241 g, 0.860 mmol) to provide the product (0.102 g, 42.0%).

### Step C: 3-[3-Amino-1-(4-fluoro-phenyl)-propyl]-N-methyl-benzamide

The product from Step B is dissolved in THF (2 mL) and cooled to 0° C. LiAlH₄ is added dropwise to the reaction mixture over the course of 5 minutes and stirred for 2 hours. The reaction is quenched by the slow addition of solid sodium sulfate decahydrate over 10 min at 0° C. The resulting slurry is stirred at 0° C., warmed to room temperature and allowed to stir for another 3 hours. The reaction is filtered through diatomaceous earth and the solid washed with THF. The resulting solutions are combined, evaporated in vacuo, and purified (flash chromatography, MeOH in dichloromethane to provide the desired product (0.055 g, 54.2%).

### Step D: N-[3-(4-Fluoro-phenyl)-3-(3-methylcarbamoyl-phenyl)-propyl]-6-(2-pyrrolidin-1-yl-ethyl)-nicotinamide

The title compound is prepared and purified (preparative TLC, MeOH in dichloromethane) using the procedure from Example 67, starting from 6-pyrrolidin-1-yl-ethyl)-nicotinic acid (0.065 g, 0.295 mmol) and the product of Example 108, Step C (0.064 g, 0.224 mmol), EDC (0.110 g, 0.577 mmol), HOBT (0.076 g, 0.562 mmol) and Hünig's base (200 μL) to provide the desired product (0.007 g, 4.900%) LCMS: 489.32 (M+H⁺).

### EXAMPLE 109

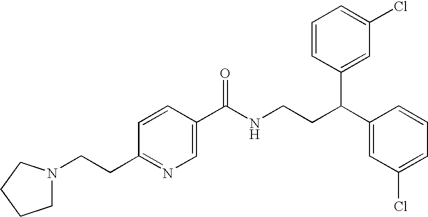

### N-[3,3-Bis-(3-chloro-phenyl)-propyl]-6-(2-pyrrolidin-1-yl-ethyl)-nicotinamide

### Step A: 3,3-Bis-(3-chloro-phenyl)-2-cyano-acrylic acid ethyl ester

A solution of 3-chlorophenylmagnesium bromide in THF (0.5 M, 44.2 mL, 22.1 mmol) is added with vigorous stirring into a solution of (Z)-3-(3-chloro-phenyl)-2-cyano-acrylic acid ethyl ester (4.00 g, 17.000 mmol) in anhydrous toluene (500 mL). The mixture is stirred for 4 hours under reflux and it is monitored by TLC until the starting material is all consumed. The mixture is then poured into crushed ice and the precipitate formed is dissolved by the addition of HCl (5%). The organic layer is then separated and the aqueous layer is extracted with ether (2×50 mL). The organic layer is then washed with aqueous sodium bicarbonate, water and dried over anhydrous sodium sulfate. Evaporation in vacuo gives the desired product (6.00 g, 100% crude).

### Step B: 2-Carbamoyl-3,3-bis-(3-chloro-phenyl)-acrylic acid

The product from Step A (17.00 mmol) is heated at reflux with aqueous NaOH (5%, 500 mmol) for two hours. It is then cooled to room temperature and washed with diethylether, acidified with HCl (5%) and extracted with ethyl acetate. The ethyl acetate layer is dried over anhydrous sodium sulfate and evaporated in vacuo to give the desired product as a pale yellow solid (4.40 g, 76.0%).

### Step C: 3,3-Bis-(3-chloro-phenyl)-propionamide

To the product from Step B (4.400 g, 13.0 mmol) in DMSO (60 mL), is added lithium chloride (1.100 g, 26.00 mmol) and the mixture is heated at reflux until the completion of the reaction (TLC monitoring). The reaction is cooled to room temperature and water (80 mL) and ethyl acetate (80 mL) are added to the mixture and shaken vigorously. The ethyl acetate layer is drained and the water layer is washed with ethyl acetate (40 mL). The combined organic layers are dried over sodium sulfate. The volatiles are removed in vacuo and purified (flash chromatography) to give the desired product (2.95 g, 76.0%).

### Step D: 33-Bis-(3-chloro-phenyl)-propylamine

To a solution of LiAlH₄ in ether (1M, 20 mL) cooled to 0° C., is added dropwise, the cold solution of the product from Step C (2.9 g, 10 mmol) in THF (20 mol, 0° C.). The mixture is stirred for 5 hours and allowed to reach the room temperature. It is then heated at reflux on a water bath. Purification of the crude mixture (HPLC) provides the desired amine.

### Step E: N-[3,3-Bis-(3-chloro-phenyl)-propyl]-6-(2-pyrrolidin-1-yl-ethyl)-nicotinamide

The title compound is prepared and purified (preparative TLC, MeOH in dichloromethane) using the procedure from Example 67, starting with 6-(pyrrolidin-yl-ethyl)-nicotinic acid (0.065 g, 0.295 mmol), the product of Step D (0.087 g, 0.311 mmol), EDC (0.110 g, 0.577 mmol), HOBT (0.076 g, 0.562 mmol) and Hünig's base (200 μL), to provide the desired product (0.071 g, 49.9%). LCMS: 483.452 (M+H⁺)

### EXAMPLE 110

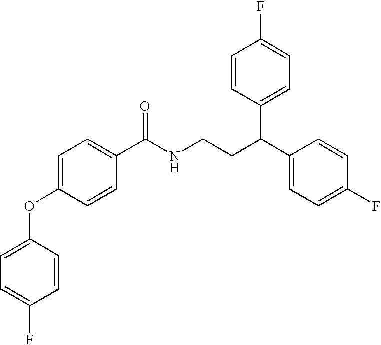

### N-[3,3-Bis-(4-fluoro-phenyl)-propyl]-4-(4-fluoro-phenoxy)-benzamide

The title compound is prepared and purified (flash chromatography, MeOH in dichloromethane) using the procedure from Example 106, starting with 4-4-fluoro-phenoxy-benzoic acid (0.104 g, 0.450 mmol), 3,3-bis-(4-fluoro-phenyl)-propyl amine (0.111 g, 0.450 mmol), EDC (1.392 g, 7.3 mmol), HOBT (0.986 g, 7.3 mmol) and Hünig's base (1.292 g, 10 mmol), to provide the desired product (0.160 g, 77.0%). LCMS: 462.26(M+H⁺).

### EXAMPLE 111

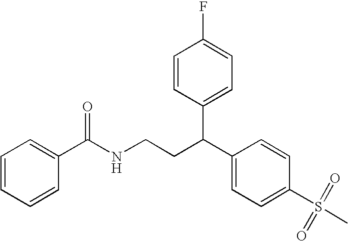

### N-[3-(4-Fluoro-phenyl)-3-(4-methanesulfonyl-phenyl)-propyl]-benzamide

To the solution of product of Example 102, Step B (0.170, 0.553 mmol), and triethylamine (240 μL) in dichloromethane, is added benzoyl chloride (0.096 μL, 0.683 mmol) dropwise and the mixture is stirred for 1.5 hours at room temperature. The mixture is then poured into water and ether and the layers separated. The ether layer is washed with sodium bicarbonate solution and brine, dried over magnesium sulfate and evaporated in vacuo. The product is then purified (flash chromatography, MeOH in dichloromethane) to provide the desired product (110 mg 50%). LCMS: 412.23 (M+H⁺).

### EXAMPLE 112

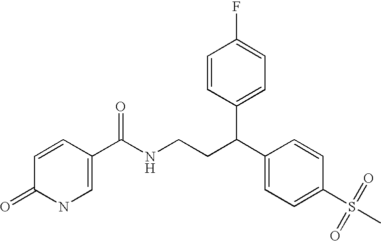

### 6-Oxo-1,6-dihydro-pyridine-3-carboxylic acid [3-(4-fluoro-phenyl)-3-(4-methanesulfonyl-phenyl)-propyl]-amide

To a solution of 6-hydroxy nicotinic acid (0.139 g, 0.999 mmol) in 2.5 mL dichloromethane is added thionyl chloride solution (1.8 mL) and the mixture is heated at reflux for 2 hours. Thionyl chloride is then removed in vacuo and the resulting oil is dissolved in dichloromethane and cooled to 0° C. Triethylamine (240 μL) and the product of Example 102, Step B, (0.170 g, 0.553 mmol) are added to the mixture and the reaction is warmed to room temperature and allowed to stir for 2 hours. The reaction mixture is poured into water and the organic layer is extracted several times with ether. The ether layers are combined and washed with brine and sodium bicarbonate, dried over magnesium sulfate and evaporated to in vacuo. The crude product is purified (flash chromatography, MeOH in dichloromethane), to provide the desired product (0.107 g, 25%). LCMS: 429.22 (M+H⁺).

### EXAMPLE 113

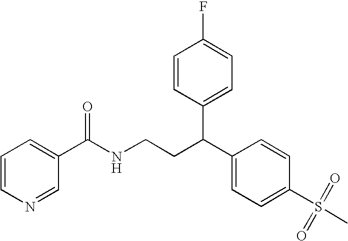

### N-[3-(4-Fluoro-phenyl)-3-(4-methanesulfonyl-phenyl)-propyl]-nicotinamide

The title compound is prepared and purified (flash chromatography, MeOH in dichloromethane) using the procedure from Example 106, starting with nicotinic acid (0.071 g, 0.577 mmol), the product of Example 102, step B, (0.170 g, 0.553 mmol), EDC (0.210 g, 1.101 mmol), HOBT (0.150 g, 1.110 mmol) and Hünig's base (280 μL, 2.166 mmol), to provide the desired product (0.140 g, 58.9%). LCMS: 413.27 (M+H⁺).

### EXAMPLE 114

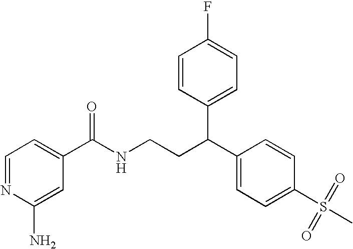

### 2-Amino-N-[3-(4-fluoro-phenyl)-3-(4-methanesulfonyl-phenyl)-propyl]-isonicotinamide

The title compound is prepared and purified (flash chromatography, MeOH in dichloromethane) using the procedure from Example 106, starting with 2-amino-isonicotinic acid (0.076 g, 0.553 mmol), the product of Example 102, Step B, (0.170 g, 0.553 mmol), EDC (0.210 g, 1.101 mmol), HOBT (0.150 g, 1.110 mmol) and Hünig's base (280 μL, 2.166 mmol), to provide the desired product (0.063 g, 26.6%). LCMS: 428.27 (M+H⁺)

### EXAMPLE 115

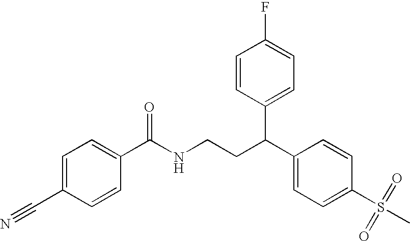

### 4-Cyano-N-[3-(4-fluoro-phenyl)-3-(4-methanesulfonyl-phenyl)-propyl]-benzamide

The title compound is prepared and purified (flash chromatography, MeOH in dichloromethane) using the procedure from Example 106, starting with 4-cyano, benzoic acid (0.081 g, 0.553 mmol), the product of Example 102, Step B, (0.170 g, 0.553 mmol), EDC (0.190 g, 1.000 mmol), HOBT (0.135 g, 1.000 mmol) and Hünig's base (516 μL, 4.000 mmol), to provide the desired product (0.041 g, 17.0%). LCMS: 437.04.

### EXAMPLE 116

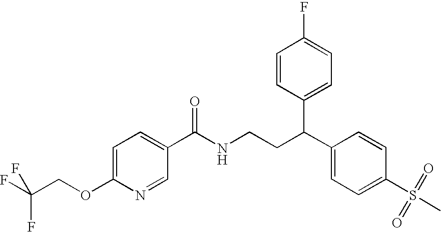

### N-[3-(4-Fluoro-phenyl)-3-(4-methanesulfonyl-phenyl)-propyl]-4-(2,2,2-trifluoro-ethoxy)-benzamide

The title compound is prepared and purified (flash chromatography, MeOH in dichloromethane) using the procedure from Example 106, starting with 6-(2,2,2-trifluoro-ethoxy)-nicotinic acid (0.122 g, 0.553 mmol), the product of Example 102, Step B, (0.170 g, 0.553 mmol), EDC (0.210 g, 1.101 mmol), HOBT (0.150 g, 1.110 mmol) and Hünig's base (280 μL, 2.166 mmol), to provide the desired product (0.089 g, 31.5%). LCMS: 511.06 (M+H⁺).

### EXAMPLE 117

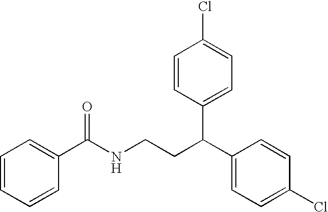

### N-[3,3-Bis-(4-chloro-phenyl)-propyl]-benzamide

The title compound is prepared and purified (flash chromatography, MeOH in dichloromethane) using the procedure from Example 106, starting with benzoic acid (0.096 g, 0.786 mmol), product of Example 105, Step A, (0.250 g, 0.790 mmol), EDC (0.303 g, 1.589 mmol), HOBT (0.220 g, 1.628 mmol) and Hünig's base (500 μL, 3.869 mmol), to provide the desired product (0.234 g, 77.5%). LCMS: 384.02 (M+H⁺).

### EXAMPLE 118

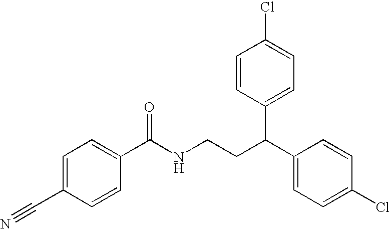

### N-[3,3-Bis-(4-chloro-phenyl)-propyl]-4-cyano-benzamide

The title compound is prepared and purified (flash chromatography, MeOH in dichloromethane) using the procedure from Example 106, starting with 4-cyano benzoic acid (0.117 g, 0.795 mmol), the product of Example 105, Step A, (0.250 g, 0.790 mmol), EDC (0.303 g, 1.589 mmol), HOBT (0.220 g, 1.628 mmol) and Hünig's base (500 μL, 3.869 mmol), to provide the desired product (0.034 g, 10.4%). LCMS: 409.04 (M+H).

### EXAMPLE 119

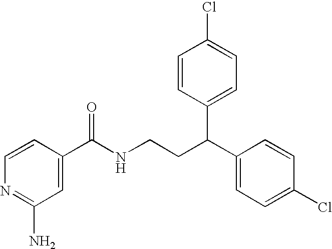

### 2-Amino-N-[3,3-bis-(4-chloro-phenyl)-propyl]-isonicotinamide

The title compound is prepared and purified (flash chromatography, MeOH in dichloromethane) using the procedure from Example 106, starting from 2-amino-isonicotinic acid (0.110 g, 0.796 mmol), the product of Example 105, Step A, (0.250 g, 0.790 mmol), EDC (0.303 g, 1.589 mmol), HOBT (0.220 g, 1.628 mmol) and Hünig's base (500 μL, 3.869 mmol), to provide the desired product (0.152 g, 47.7%). LCMS: 400.04 (M+H⁺).

### EXAMPLE 120

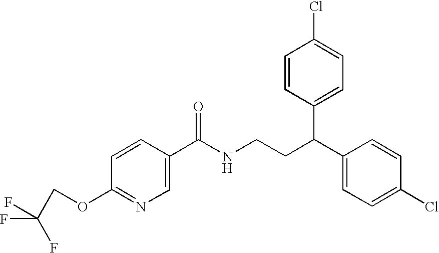

### 6-(2,2,2-Trifluoro-ethoxy)-1,6-dihydro-pyridine-3-carboxylic acid [3,3-bis-(4-chloro-phenyl)-propyl]-amide

The title compound is prepared and purified (flash chromatography, MeOH in dichloromethane) using the procedure from Example 106, starting from 6-(2,2,2-trifluoro-ethoxy)-nicotinic acid (0.175 g, 0.791 mmol), the product of Example 105, Step A, (0.250 g, 0.790 mmol), EDC (0.303 g, 1.589 mmol), HOBT (0.220 g, 1.628 mmol) and Hünig's base (500 μL, 3.869 mmol), to provide the desired product (0.250 g, 65.4%). LCMS: 484.315, (M+H⁺).

### EXAMPLE 121

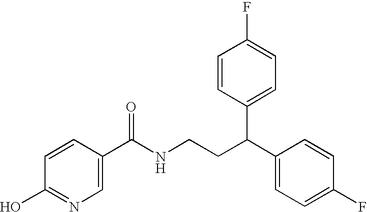

### 6-Oxo-1,6-dihydro-pyridine-3-carboxylic acid [3,3-bis-(4-fluoro-phenyl)-propyl]-amide

The title compound is prepared and purified (preparative TLC, 5% MeOH in dichloromethane) using the procedure from Example 1, starting from 3,3-bis-(4-fluoro-phenyl)-propylamine (0.04 g, 0.170 mmol) and 6-hydroxy nicotinic acid (0.0236, 0.170 mmol), to give the desired product (0.003, 4.8%). LCMS: 369.37 (M+H⁺).

### EXAMPLE 122

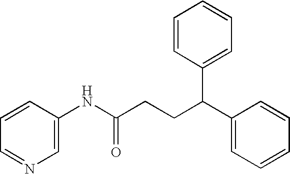

### 4,4-Diphenyl-N-pyridin-3-yl-butyramide

### Step A: 3,3-Diphenyl-1-propionic acid

To a solution of phenyl-butyrolactone (1.620 g, 9.988 mmol) in 50 mL dry benzene is added aluminum trichloride (1.460 g, 10.940 mmol). The reaction is stirred at room temperature for 5 hours followed by the addition of 2 M aqueous HCl. The organic layer is extracted twice with ether and washed twice with water, dried over sodium sulfate and evaporated in vacuo which provides the desired product (2.30 g, 95.8%).

### Step B: 4,4-Diphenyl-butyryl chloride

The product from Step A (0.730 g, 3.038 mmol) is dissolved in 5 mL dichloromethane followed by the addition of one drop of DMF and oxalyl chloride (0.530 mL, 6.075 mmol). The mixture is stirred at room temperature for one hour and evaporated in vacuo to give the desired product. (0.77 g, 95.0%)

### Step C: 4,4-Diphenyl-N-pyridin-3-yl-butyramide

To a solution of 3-amino-pyridine (0.100 g, 1.063 mmol) in dichloromethane is added the acid chloride from Step B (0.900 mmol), and triethylamine (0.250 mL) in 2.500 mL dichloromethane. Reaction is stirred at room temperature for 16 hours. It is then evaporated in vacuo and purified using flash chromatography (0-4% methanol/dichloromethane, then 10% methanol/dichloromethane) to give the desired product (0.186 g, 55.3%). LCMS: 317.47 (M+H⁺).

### EXAMPLE 123

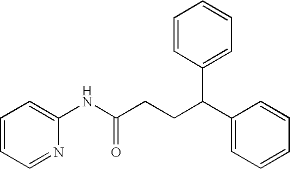

### 4,4-Diphenyl-N-pyridin-2-yl-butyramide

### Step A: 4,4-Diphenyl-butyric acid

A solution of phenyl butyrolactone (1.62 g, 9.99 mmol) in dry benzene (50 mL) is added dropwise to aluminum chloride (1.46 g, 10.95 mmol), and the mixture is stirred at room temperature for 5 hours. Aqueous HCl (2 M) is added to the mixture and the organic layer is extracted twice, washed twice with water, dried over sodium sulfate and evaporated in vacuo to give desired product (2.20 g, 91.7%).

### Step B: 4,4-Diphenyl-N-pyridin-2-yl-butyramide

To the solution of the product from Step A (0.100 mg, 0.416 mmol) in dichloromethane (2.5 mL) is added EDC (0.160 mg, 0.822 mmol), and HOBT (111 mg, 0.822 mmol). The reaction is stirred at room temperature for 15 minutes and 2-aminopyridine (0.050 mg, 0.531 mmol), and triethylamine (72 μL) are added to the mixture. The reaction is stirred at room temperature for 16 hours. The mixture is then diluted with dichloromethane, washed twice with water dried over sodium sulfate and evaporated in vacuo. The resulting oil is purified (flash chromatography, 0-25% EtOAC: hexanes, followed by preparative TLC 4:6 EtOAc: hexanes) to give the desired product (0.011 g, 8.4%). LCMS: 317.46 (M+H⁺).

### EXAMPLE 124

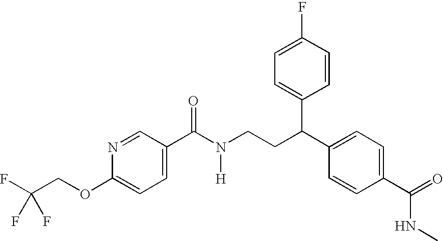

### N-[3-(4-Fluoro-phenyl)-3-(4-methylcarbamoyl-phenyl)-propyl]-6-(2,2,2-trifluoro-ethoxy)-nicotinamide

To a solution of 6-(2,2,2-trifluoro-ethoxy)-nicotinic acid (1 mmol) in DMF (4 mL) is added 4-[3-amino-1-(4-fluoro-phenyl)-propyl]-N-methyl-benzamide (1 mmol) followed by the addition of HOBT (2 mmol), EDC (2 mmol) and diisopropylethylamine (4 mmol). The reaction is stirred overnight. The mixture is diluted with water and the product is extracted with EtOAc. The organic extract is dried over magnesium sulfate and evaporated in vacuo. The resulting oil is purified (flash chromatography, MeOH/dichloromethane) to give the desired product (0.173 g, 45.7%). LCMS: 489.99 (M+H⁺).

### EXAMPLE 125

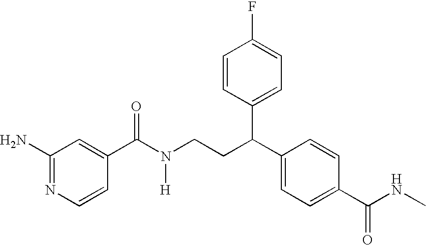

### 2-Amino-N-[3-(4-fluoro-phenyl)-3-(4-methylcarbamoyl-phenyl)-propyl]-isonicotinamide

To a solution of 2-amino-isonicotinic acid (1 mmol) in DMF (4 mL) is added 4-[3-amino-1-(4-fluoro-phenyl)-propyl]-N-methyl-benzamide (1 mmol) followed by the addition of HOBT (2 mmol), EDC (2 mmol) and diisopropylethylamine (4 mmol). The reaction is stirred overnight. The mixture is diluted with water and the product is extracted with EtOAc. The organic extract is dried over magnesium sulfate and evaporated in vacuo. The resulting oil is purified (flash chromatography, MeOH/DCM) to give the desired product (0.139 g, 44.2%). LCMS: 407.37 (M+H⁺).

### EXAMPLE 126

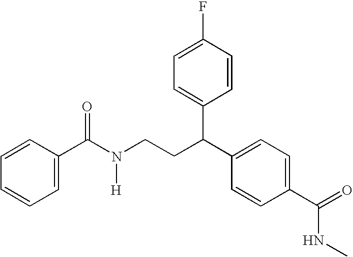

### 4-[3-Benzoylamino-1-(4-fluoro-phenyl)-propyl]-N-methyl-benzamide

To a solution of benzoic acid (1 mmol) in DMF (4 mL) is added 4-[3-amino-1-(4-fluoro-phenyl)-propyl]-N-methyl-benzamide (1 mmol) followed by the addition of HOBT (2 mmol), EDC (2 mmol) and diisopropylethylamine (4 mmol). The reaction is stirred overnight. The mixture is diluted with water and the product is extracted with EtOAc. The organic extract is dried over magnesium sulfate and evaporated in vacuo. The resulting oil is purified (flash chromatography, MeOH/dichloromethane) to give the desired product (0.179 g, 59.2%). LCMS: 435.11 (M+H⁺).

### EXAMPLE 127

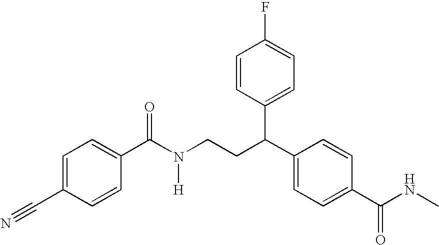

### N-[3-(4-Fluoro-phenyl)-3-(4-methylcarbamoyl-phenyl)-propyl]-6-cyanobenzamide

To a solution of 4-cyano-benzoic acid (1 mmol) in DMF (4 mL) is added 4-[3-amino-1-(4-fluoro-phenyl)-propyl]-N-methyl-benzamide, (1 mmol) followed by the addition of HOBT (2 mmol), EDC (2 mmol) and diisopropylethylamine (4 mmol). The reaction is stirred overnight. The mixture is diluted with water and the product is extracted with EtOAc. The organic extract is dried over magnesium sulfate and evaporated in vacuo. The resulting oil is purified (flash chromatography, MeOH: dichloromethane) to give the desired product (0.204 g, 63.4%). LCMS: 415.97 (M+H⁺).

### EXAMPLE 128

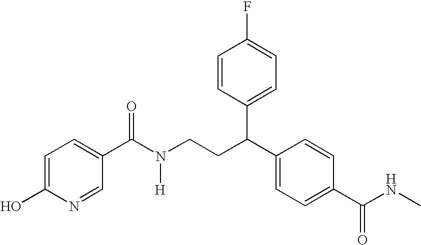

### N-[3-(4-Fluoro-phenyl)-3-(4-methylcarbamoyl-phenyl)-propyl]-6-hydroxy-nicotinamide

To a solution of 6-hydroxy-nicotinic acid (1 mmol) in DMF (4 mL), is added 4-[3-amino-1-(4-fluoro-phenyl)-propyl]-N-methyl-benzamide (1 mmol), followed by the addition of HOBT (2 mmol), EDC (2 mmol) and diisopropylethylamine (4 mmol). The reaction is stirred overnight. The mixture is diluted with water and the product is extracted using EtOAc. The organic extract is dried over magnesium sulfate and evaporated in vacuo. The resulting oil is purified (flash chromatography, MeOH: dichloromethane) to give the desired product (0.055 g, 17.4%). LCMS: 408.03 (M+H⁺).

In addition, the following compounds may be prepared by any of the above procedures:

| | |
|---|---|
| 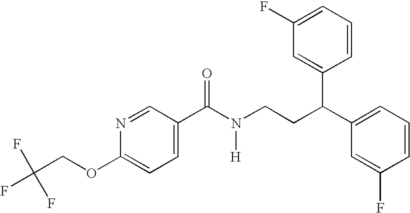 | N-[3,3-Bis-(3-fluoro-phenyl)-propyl]-6- (2,2,2-trifluoro-ethoxy)-nicotinamide |
| 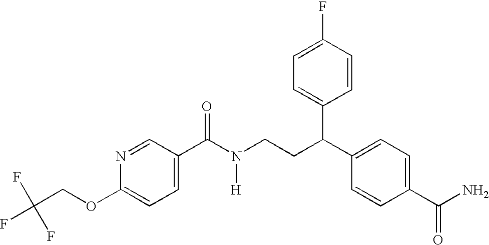 | N-[3-(4-Carbamoyl-phenyl)-3-(4-fluoro- phenyl)-propyl]-6-(2,2,2-trifluoro- ethoxy)-nicotinamide |
| 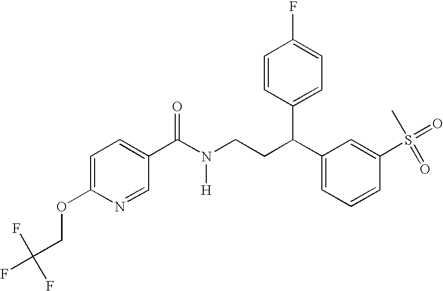 | N-[3-(4-Fluoro-phenyl)-3-(3- methanesulfonyl-phenyl)-propyl]-6- (2,2,2-trifluoro-ethoxy)-nicotinamide |
| 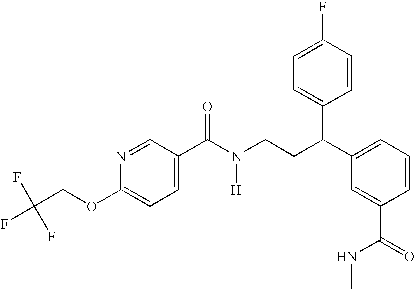 | N-[3-(4-Fluoro-phenyl)-3-(3- methylcarbamoyl-phenyl)-propyl]-6- (2,2,2-trifluoro-ethoxy)-nicotinamide |
| 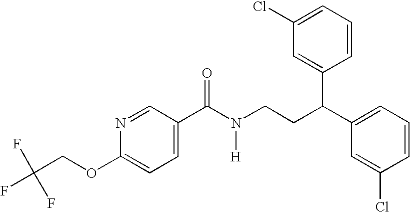 | N-[3,3-Bis-(3-chloro-phenyl)-propyl]-6- (2,2,2-trifluoro-ethoxy)-nicotinamide |
| 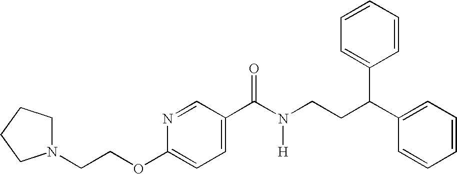 | N-(3,3-Diphenyl-propyl)-6-(2-pyrrolidin- 1-yl-ethoxy)-nicotinamide |
| 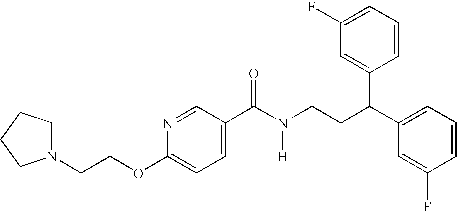 | N-[3,3-Bis-(3-fluoro-phenyl)-propyl]-6- (2-pyrrolidin-1-yl-ethoxy)-nicotinamide |
| 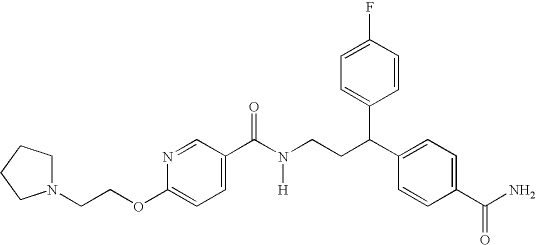 | N-[3-(4-Carbamoyl-phenyl)-3-(4-fluoro- phenyl)-propyl]-6-(2-pyrrolidin-1-yl- ethoxy)-nicotinamide |
| 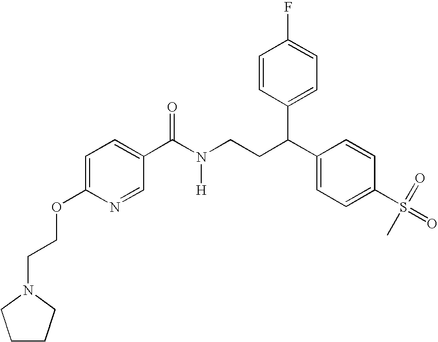 | N-[3-(4-Fluoro-phenyl)-3-(4- methanesulfonyl-phenyl)-propyl]-6-(2- pyrrolidin-1-yl-ethoxy)-nicotinamide |
| 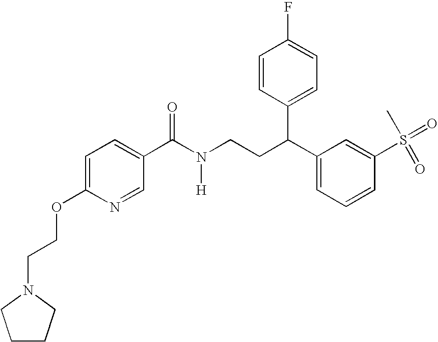 | N-[3-(4-Fluoro-phenyl)-3-(3- methanesulfonyl-phenyl)-propyl]-6-(2- pyrrolidin-1-yl-ethoxy)-nicotinamide |
| 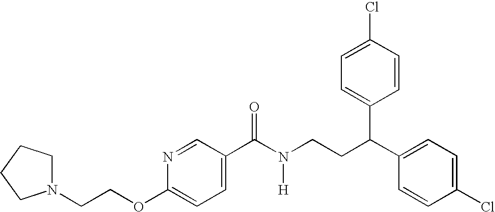 | N-[3,3-Bis-(4-chloro-phenyl)-propyl]-6- (2-pyrrolidin-1-yl-ethoxy)-nicotinamide |
| 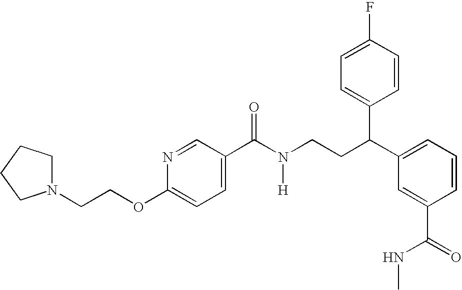 | N-[3-(4-Fluoro-phenyl)-3-(3- methylcarbamoyl-phenyl)-propyl]-6-(2- pyrrolidin-1-yl-ethoxy)-nicotinamide |
| 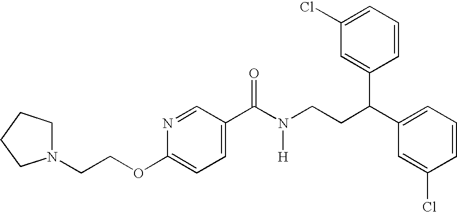 | N-[3,3-Bis-(3-chloro-phenyl)-propyl]-6- (2-pyrrolidin-1-yl-ethoxy)-nicotinamide |
| 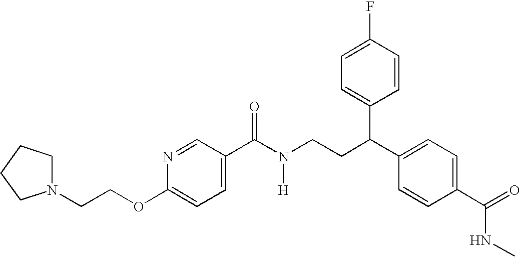 | N-[3-(4-Fluoro-phenyl)-3-(4- methylcarbamoyl-phenyl)-propyl]-6-(2- pyrrolidin-1-yl-ethoxy)-nicotinamide |
| 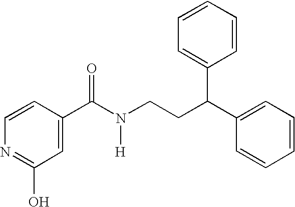 | N-(3,3-Diphenyl-propyl)-2-hydroxy- isonicotinamide |
| 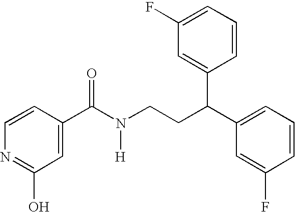 | N-[3,3-Bis-(3-fluoro-phenyl)-propyl]-2- hydroxy-isonicotinamide |
| 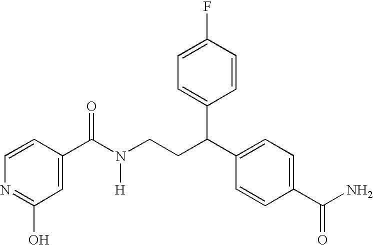 | N-[3-(4-Carbamoyl-phenyl)-3-(4-fluoro- phenyl)-propyl]-2-hydroxy- isonicotinamide |
| 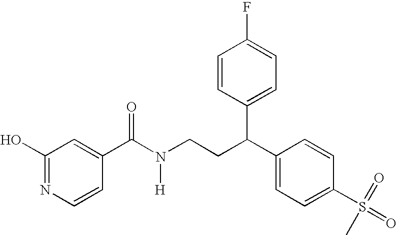 | N-[3-(4-Fluoro-phenyl)-3-(4- methanesulfonyl-phenyl)-propyl]-2- hydroxy-isonicotinamide |
| 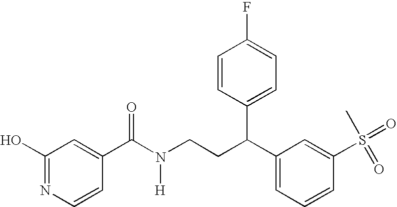 | N-[3-(4-Fluoro-phenyl)-3-(3- methanesulfonyl-phenyl)-propyl]-2- hydroxy-isonicotinamide |
| 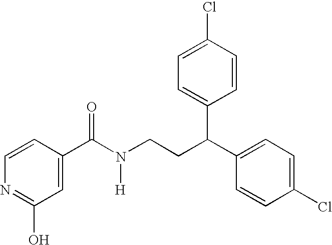 | N-[3,3-Bis-(4-chloro-phenyl)-propyl]-2- hydroxy-isonicotinamide |
| 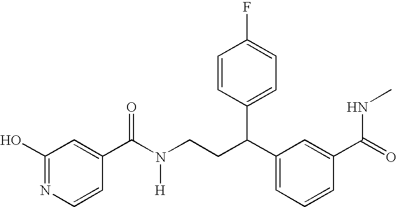 | N-[3-(4-Fluoro-phenyl)-3-(3- methylcarbamoyl-phenyl)-propyl]-2- hydroxy-isonicotinamide |
| 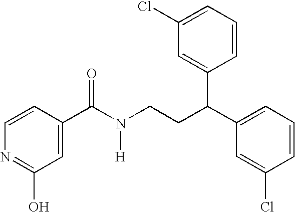 | N-[3,3-Bis-(3-chloro-phenyl)-propyl]-2- hydroxy-isonicotinamide |
| 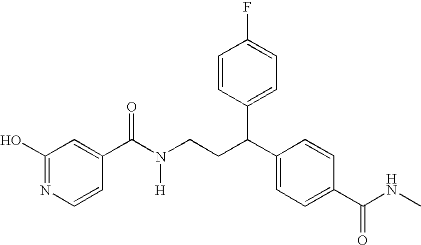 | N-[3-(4-Fluoro-phenyl)-3-(4- methylcarbamoyl-phenyl)-propyl]-2- hydroxy-isonicotinamide |
| 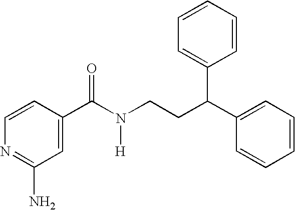 | 2-Amino-N-(3,3-diphenyl-propyl)- isonicotinamide |
| 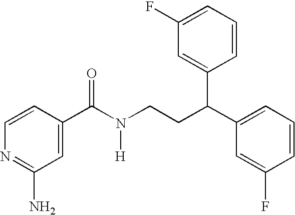 | 2-Amino-N-[3,3-bis-(3-fluoro-phenyl)- propyl]-isonicotinamide |
| 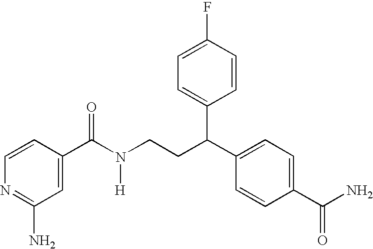 | 2-Amino-N-[3-(4-carbamoyl-phenyl)-3- (4-fluoro-phenyl)-propyl]- isonicotinamide |
| 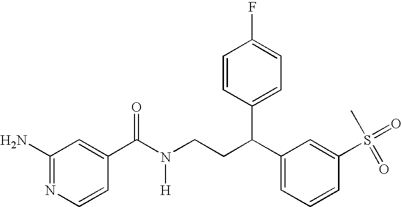 | 2-Amino-N-[3-(4-fluoro-phenyl)-3-(3- methanesulfonyl-phenyl)-propyl]- isonicotinamide |
| 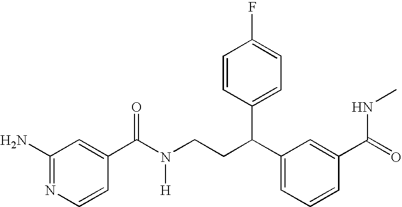 | 2-Amino-N-[3-(4-fluoro-phenyl)-3-(3- methylcarbamoyl-phenyl)-propyl]- isonicotinamide |
| 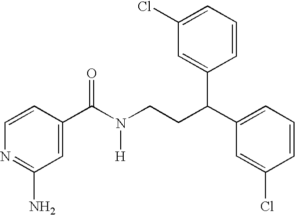 | 2-Amino-N-[3,3-bis-(3-chloro-phenyl)- propyl]-isonicotinamide |
| 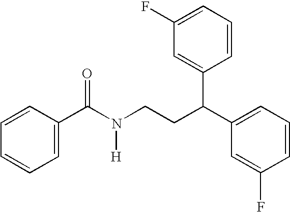 | N-[3,3-Bis-(3-fluoro-phenyl)-propyl]- benzamide |
| 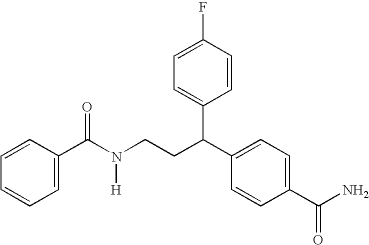 | 4-[3-Benzoylamino-1-(4-fluoro-phenyl)- propyl]-benzamide |
| 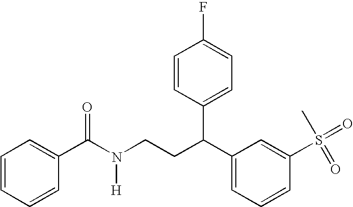 | N-[3-(4-Fluoro-phenyl)-3-(3- methanesulfonyl-phenyl)-propyl]- benzamide |
| 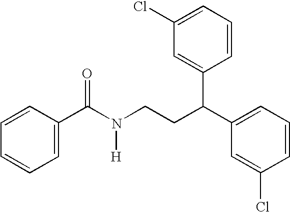 | N-[3,3-Bis-(3-chloro-phenyl)-propyl]- benzamide |
| 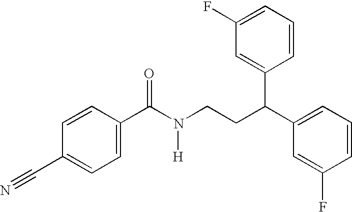 | N-[3,3-Bis-(3-fluoro-phenyl)-propyl]-4- cyano-benzamide |
| 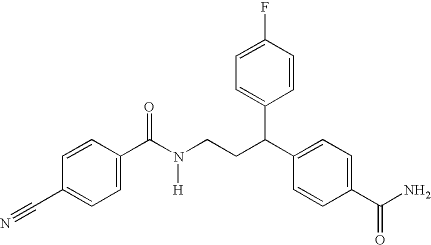 | 4-[3-Benzoylamino-1-(4-fluoro-phenyl)- propyl]-4-cyano-benzamide |
| 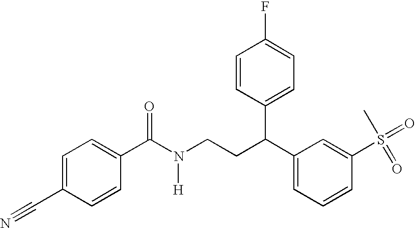 | 4-Cyano-N-[3-(4-fluoro-phenyl)-3-(3- methanesulfonyl-phenyl)-propyl]- benzamide |
| 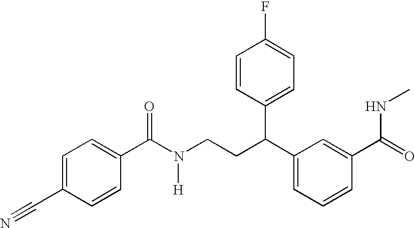 | 3-[3-Benzoylamino-1-(4-fluoro-phenyl)- propyl]-N-methyl-4-cyano-benzamide |
| 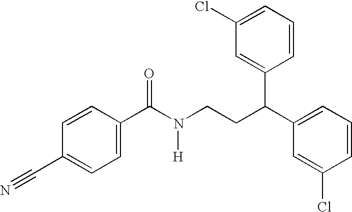 | N-[3,3-Bis-(3-chloro-phenyl)-propyl]-4- cyano-benzamide |
| 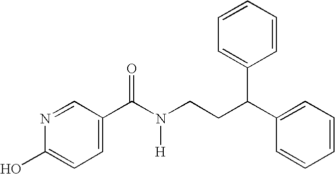 | N-(3,3-Diphenyl-propyl)-6-hydroxy- nicotinamide |
| 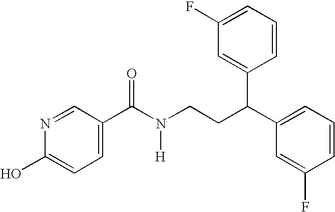 | N-[3,3-Bis-(3-fluoro-phenyl)-propyl]-6- hydroxy-nicotinamide |
| 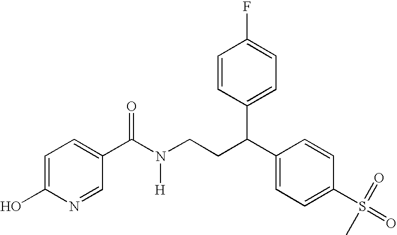 | N-[3-(4-Fluoro-phenyl)-3-(4- methanesulfonyl-phenyl)-propyl]-6- hydroxy-nicotinamide |
| 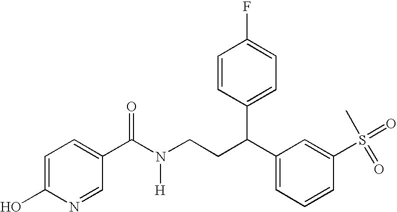 | N-[3-(4-Fluoro-phenyl)-3-(3- methanesulfonyl-phenyl)-propyl]-6- hydroxy-nicotinamide |
| 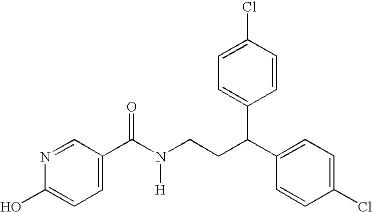 | N-[3,3-Bis-(4-chloro-phenyl)-propyl]-6- hydroxy-nicotinamide |
| 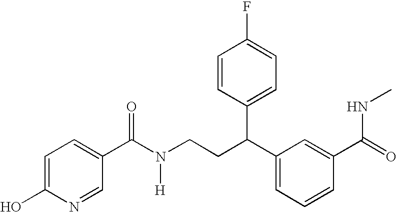 | N-[3-(4-Fluoro-phenyl)-3-(3- methylcarbamoyl-phenyl)-propyl]-6- hydroxy-nicotinamide |
| 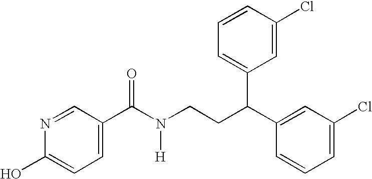 | N-[3,3-Bis-(3-chloro-phenyl)-propyl]-6- hydroxy-nicotinamide |

### Methods of Use

In accordance with the invention, there are provided methods of using the compounds as desrcribed herein and their pharmaceutically acceptable derivatives. The compounds used in the invention prevent the degradation of sEH substrates that have beneficial effects or prevent the formation of metabolites that have adverse effects. The inhibition of sEH is an attractive means for preventing and treating a variety of cardiovascular diseases or conditions e.g., endothelial dysfunction. Thus, the methods of the invention are useful for the treatment of such conditions. These encompass diseases including, but not limited to, type 1 and type 2 diabetes, insulin resistance syndrome, hypertension, atherosclerosis, coronary artery disease, angina, ischemia, ischemic stroke, Raynaud's disease and renal disease.

For therapeutic use, the compounds may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds described herein may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the above-described compounds include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

The term “patient” includes both human and non-human mammals.

The term “effective amount” means an amount of a compound according to the invention which, in the context of which it is administered or used, is sufficient to achieve the desired effect or result. Depending on the context, the term effective amount may include or be synonymous with a pharmaceutically effective amount or a diagnostically effective amount.

The terms “pharmaceutically effective amount” or “therapeutically effective amount” means an amount of a compound according to the invention which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the invention which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the invention, and the age, body weight, general health, sex, and diet of the patient. Such a therapeutically effective amount can be determined routinely by one of ordinary skill in the art having regard to their own knowledge, the prior art, and this disclosure.

The term “diagnostically effective amount” means an amount of a compound according to the invention which, when used in a diagnostic method, apparatus, or assay, is sufficient to achieve the desired diagnostic effect or the desired biological activity necessary for the diagnostic method, apparatus, or assay. Such an amount would be sufficient to elicit the biological or medical response in a diagnostic method, apparatus, or assay, which may include a biological or medical response in a patient or in a in vitro or in vivo tissue or system, that is sought by a researcher or clinician. The amount of a compound according to the invention which constitutes a diagnostically effective amount will vary depending on such factors as the compound and its biological activity, the diagnostic method, apparatus, or assay used, the composition used for administration, the time of administration, the route of administration, the rate of excretion of the compound, the duration of administration, drugs and other compounds used in combination with or coincidentally with the compounds of the invention, and, if a patient is the subject of the diagnostic administration, the age, body weight, general health, sex, and diet of the patient. Such a diagnostically effective amount can be determined routinely by one of ordinary skill in the art having regard to their own knowledge, the prior art, and this disclosure.

The terms “treating” or “treatment” mean the treatment of a disease-state in a patient, and include:
(i) preventing the disease-state from occurring in a patient, in particular, when such patient is genetically or otherwise predisposed to the disease-state but has not yet been diagnosed as having it;
(ii) inhibiting or ameliorating the disease-state in a patient, i.e., arresting or slowing its development; or
(iii) relieving the disease-state in a patient, i.e., causing regression or cure of the disease-state.

### In Vitro Assay for Inhibition of hsEH

This high throughput screen identifies compounds that inhibit the interaction of human soluble epoxide hydrolase (sEH) with a tetramethyl rhodamine (TAMRA)-labeled probe. The UHTS employs the Zymark Allegro modular robotic system to dispense reagents, buffers, and test compounds into either 96-well or 384-well black microtiter plates (from Costar). The assay buffer is: 20 mM TES, 200 mM NaCl, 0.05% w/v CHAPS, 1 mM TCEP, pH=7.0. Test compounds dissolved in neat DMSO at 5 mg/mL are diluted to 0.5 mg/mL in neat DMSO. The 0.5 mg/mL solutions are further diluted to 30 μg/mL in assay buffer containing DMSO such that the final concentration of DMSO is 30%. For 384-well format, a mixture of 10.35 nM human sEH and 2.59 nM probe is prepared in assay buffer and 60 μL is added to each well for a final sEH concentration of 10 nM and a final probe concentration of 2.5 nM. 2.1 μL of diluted test compound is then added to each well, where the final assay concentration will be 1 μg/mL test compound and 1% DMSO. The final volume in each well is 62.1 μL. Positive controls are reaction mixtures containing no test compound; negative controls (blanks) are reaction mixtures containing 3 μM B100611349XX. For 96-well format, the final concentration of all reaction components remains the same. 135 μL sEH/probe mixture is added to wells containing 15 μL test compound so that the final well volume is 150 mL. After incubating the reaction for 30 minutes at room temperature, the plates are read for fluorescence polarization in the LJL Analyst set to 530 nm excitation, 580 nm emission, using the Rh 561 dichroic mirror.

### In Vitro Assay for Inhibition of rsEH

This screen identifies compounds that inhibit the interaction of rat soluble epoxide hydrolase (sEH) with a tetramethyl rhodamine (TAMRA)-labeled probe. The assay employs a Multimek, a Multidrop, and manual multi-channel pipettors to dispense reagents, buffers, and test compounds into 96-well black microtiter plates (Costar 3792). The assay buffer is: 20 mM TES, 200 mM NaCl, 0.05% w/v CHAPS, 1 mM TCEP, pH=7.0. Test compounds dissolved in neat DMSO at 10 mM are diluted to 1.5 mM in neat DMSO. The 1.5 mM solutions are serially diluted using 3-fold dilutions in neat DMSO in polypropylene plates. Assay buffer is added to the wells such that the compounds are diluted 10-fold and the DMSO concentration is 10%. A mixture of 11.1 nM rat sEH and 2.78 nM probe is prepared in assay buffer. 15 μL of diluted test compound is added to each well, where the final maximum assay concentration will be 3 uM test compound and 1% DMSO. 135 uL of sEH/probe mixture is added to each well for a final sEH concentration of 10 nM and a final probe concentration of 2.5 nM. The final volume in each well is 150 uL. Positive controls are reaction mixtures containing no test compound; negative controls (blanks) are reaction mixtures containing 3 uM BI00611349XX. After incubating the reaction for 30 minutes at room temperature, the plates are read for fluorescence polarization in the LJL Analyst set to 530 nm excitation, 580 nm emission, using the Rh 561 dichroic mirror.

What is claimed:

1. A method of treating hypertension comprising administering to a patient an effective amount of a compound of the formula (I): 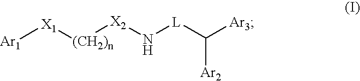
wherein:
n is 0 or 1;
X₁ is bond or a heteroatom chosen from O, S or a bond;
X₂ is —C(O)—;
L is an ethylene linking group optionally substituted by hydoxy, amino, lower alkoxy, lower alkylamino, lower alkylthio or 1-3 fluorine atoms;
Ar₁ is carbocycle, heteroaryl or heterocyclyl optionally substituted by Y;
Ar₂ and Ar₃ are carbocycle, heteroaryl or heterocyclyl each optionally substituted by one or more halogen, lower alkylS(O)ₘ, NR₂R₃—C(O)—, lower alkoxy or carboxamide;
R₁ is hydrogen or lower alkyl;
wherein the group —(CH₂)ₙ— in the formula (I) is optionally substituted by lower alkyl;
Y is chosen from
lower alkyl, lower alkoxy, lower alkenyl, lower acyl, lower alkyl(OH), —NR₂R₃;
or Y is a cyclic group chosen from heterocycle, heteroaryl and carbocycle;
each Y where possible is optionally substituted by one to three oxo, lower acyl, halogen, nitrile, lower alkylS(O)ₘ—, lower alkoxycarbonyl, NR₂R₃—C(O)—, —NR₂R₃, lower alkyl, C₃₋₆ cycloalkylC₀₋₂alkyl, hydroxy, lower alkoxy, aryloxy, arylC₀₋₄ alkyl, heteroaryl C₀₋₄ alkyl and heterocycle C₀₋₄alkyl, each above-listed heterocycle, heteroaryl and aryl group is optionally substituted by one to three hydroxy, oxo, lower alkyl, lower alkoxy, lower alkoxycarbonyl, NR₂R₃—C(O)— or lower acyl;
each R₂ and R₃ are independently hydrogen, arylC₀₋₄ alkyl, heteroaryl C₀₋₄ alkyl, heterocycle C₀₋₄alkyl, C₁₋₂ acyl, aroyl and lower alkyl optionally substituted by lower alkylS(O)ₘ—, lower alkoxy, hydroxy or mono or diC₁₋₃ alkyl amino;
or R₂ and R₃ optionally combine with the nitrogen atom to which they are attached to form a heterocyclic ring;
m is 0, 1 or 2;
or the pharmaceutically acceptable salts thereof.

2. The method according to claim 1 and wherein:
Ar₁ is cyclohexyl, phenyl; ademantyl, norbonyl,
or
heteroaryl chosen from pyridinyl, pyridinyl N-oxide, isoquinolinyl, quinolinyl, pyridazinyl and pyrimidinyl,
or
heterocyclyl chosen from piperidinyl, tetrahydropyranyl, morpholinyl, pyrrolidinyl, tetrahydrofuranyl, pyrrolidinonyl and benztriazolyl;
each Ar₁ is optionally substituted by Y;
Ar₂ and Ar₃ are each phenyl or pyridinyl optionally substituted by one or more lower alkoxy, F, Cl, lower alkylS(O)₂, lower alkyl-NH—C(O)— or carboxamide;
L is an ethylene linking group.

3. The method according to claim 2 and wherein
Ar₂ and Ar₃ are each phenyl or pyridinyl substituted by one or more lower alkoxy, F, Cl, CH₃—S(O)₂—, CH₃—NH—C(O)— or carboxamide.

4. A method of treating hypertension comprising administering to a patient an effective amount of a compound of the formula (II): 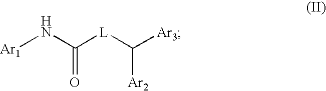
wherein:
Ar₁ is carbocycle, heteroaryl or heterocyclyl optionally substituted by Y;
Ar₂ and Ar₃ are each carbocycle optionally substituted by halogen, lower alkoxy, lower alkylS(O)ₘ, NR₂R₃—C(O)— or carboxamide;
L is an ethylene linking group optionally substituted by hydoxy, amino, lower alkoxy, lower alkylamino, lower alkylthio or 1-3 fluorine atoms;
Y is chosen from
lower alkyl, lower alkoxy, lower alkenyl, lower acyl, lower alkyl(OH), —NR₂R₃;
or Y is a cyclic group chosen from heterocycle, heteroaryl and carbocycle;
each Y where possible is optionally substituted by one to three oxo, lower acyl, halogen, nitrile, lower alkylS(O)ₘ—, lower alkoxycarbonyl, NR₂R₃—C(O)—, —NR₂R₃, lower alkyl, C₃₋₆ cycloalkylC₀₋₂alkyl, hydroxy, lower alkoxy, aryloxy, arylC₀₋₄ alkyl, heteroaryl C₀₋₄ alkyl and heterocycle C₀₋₄alkyl, each above-listed heterocycle, heteroaryl and aryl group is optionally substituted by one to three hydroxy, oxo, lower alkyl, lower alkoxy, lower alkoxycarbonyl, NR₂R₃—C(O)— or lower acyl;
each R₂ and R₃ are independently hydrogen, arylC₀₋₄ alkyl, heteroaryl C₀₋₄ alkyl, heterocycle C₀₋₄alkyl, C₁₋₂ acyl, aroyl and lower alkyl optionally substituted by lower alkylS(O)ₘ—, lower alkoxy, hydroxy or mono or diC₁₋₃ alkyl amino;
or R₂ and R₃ optionally combine with the nitrogen atom to which they are attached to form a heterocyclic ring;
m is 0, 1 or 2;
or the pharmaceutically acceptable salts thereof.

5. The method according to claim 4 and wherein:
Ar₁ is cyclohexyl, phenyl, adamantyl, norbornyl,
or
heteroaryl chosen from pyridinyl, pyridinyl N-oxide, isoquinolinyl, quinolinyl, pyridazinyl and pyrimidinyl,
or
heterocyclyl chosen from piperidinyl, tetrahydropyranyl, morpholinyl, pyrrolidinyl, tetrahydrofuranyl, pyrrolidinonyl and benztriazolyl;
each optionally substituted by Y;
Ar₂ and Ar₃ are each phenyl or pyridinyl optionally substituted by one or more lower alkoxy, F, Cl, lower alkylS(O)₂, lower alkyl-NH—C(O)— or carboxamide;
L is an ethylene linking group.

6. The method according to claim 5 and wherein:
Ar₂ and Ar₃ are each phenyl or pyridinyl substituted by one or more lower alkoxy, F, Cl, CH₃—S(O)₂—, CH₃—NH—C(O)— or carboxamide.

7. A compound of the formula (III): 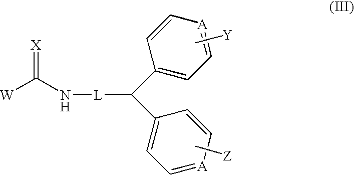
wherein:
each A is independently nitrogen or C—H such that each of the ring of which A is a member may be pyridinyl or phenyl, said pyridinyl or phenyl are optionally substituted by Y or Z;
Y and Z on their respective rings are in the meta or para position, and are independently F, Cl, Br, CN, OR, R, —S(O)₂R, —C(O)NRR or —S(O)₂NRR, wherein R is independently hydrogen or lower alkyl unsubstituted or substituted with hydroxy, amino, C₁₋₄ alkoxy, C₁₋₄ alkylamino, C₁₋₄ alkylthio, or one to three fluorine atoms;
L is an ethylene linker optionally substituted with hydroxy, amino, C₁₋₄ alkoxy C₁₋₄ alkylamino, C₁₋₄ alkylthio, or one to three fluorine atoms;
X is O or S;
W is chosen from phenyl, 2-pyridinyl, 3-pyridinyl, 4-pyridinyl, pyrazinyl, 3-pyridazinyl, 4-pyridazinyl, naphthyl, quinolinyl and isoquinolinyl each optionally with one to three substituents independently chosen from: halogen, hydroxy, amino, cyano, carboxy, carboxamido, C₁₋₄ alkyl unsubstitued or substituted with one to three halogen atoms, C₃₋₆ cycloalkyl unsubstitued or substituted with one to three halogen atoms, C₂₋₄ alkynyl, C₁₋₄ alkyloxycarbonyl, C₁₋₄ alkylamidocarbonyl, C₁₋₄ dialkylamidocarbonyl, C₁₋₄ alkylamino, C₁₋₄ dialkylamino, C₃₋₆ cycloalkylamino, di(C₃₋₆ cycloalkyl)amino, C₁₋₄ alkylsulfonyl, C₁₋₄ alkylheterocylyl, phenyl, or heterocylyl;
with the proviso that if the phenyl or pyridinyl rings possessing the aforementioned A are either unsubstituted or both substituted by halogen, then W must be substituted by any of the above-listed substituents for W;
or the pharmaceutically acceptable salts thereof.

8. A compound of the formula (IV): 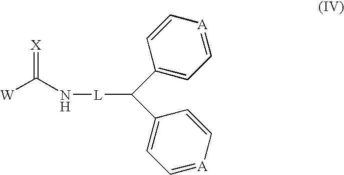
wherein for the Formula (IV), the component 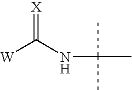
is chosen from A1-A8 in the table I below; in combination with any component 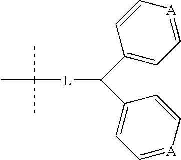
chosen from B1-B10 in the table I below;

| | 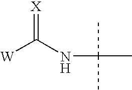 |
|---|---|
| A1 | 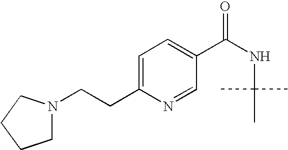 |
| A2 | 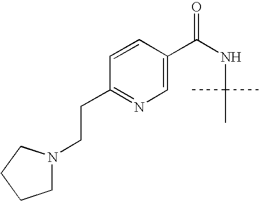 |
| A3 | 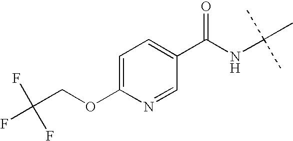 |
| A4 | 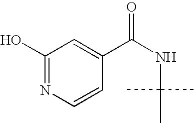 |
| A5 | 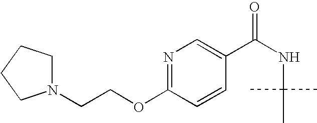 |
| A6 | 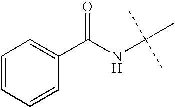 |
| A7 | 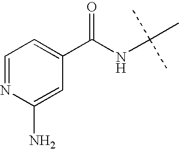 |
| A8 | 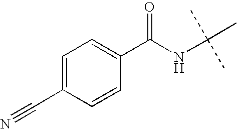 |
| A9 | |
| A10 | |
| | 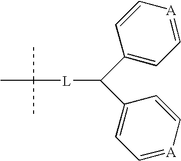 |
| B1 | 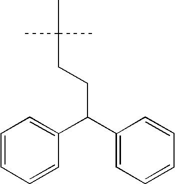 |
| B2 | 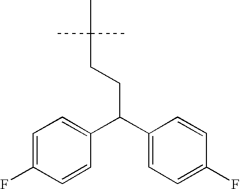 |
| B3 | 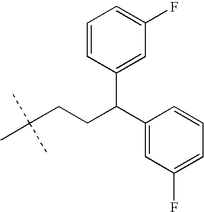 |
| B4 | 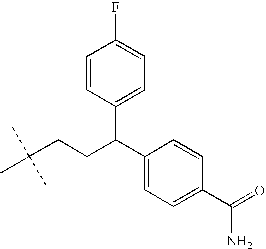 |
| B5 | 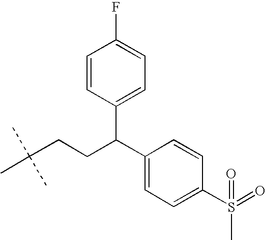 |
| B6 | 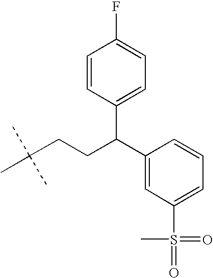 |
| B7 | 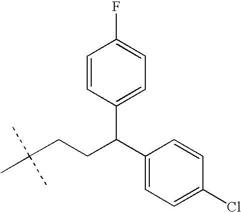 |
| B8 | 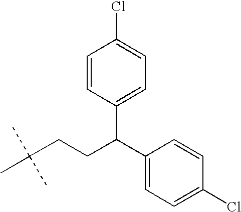 |
| B9 | 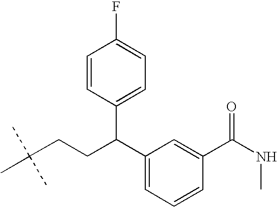 |
| B10 | 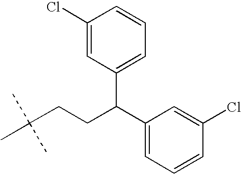 |

or the pharmaceutically acceptable salts thereof, with the proviso that if 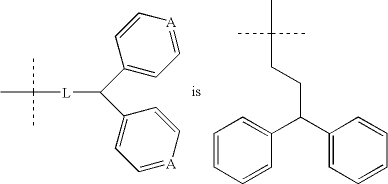
then 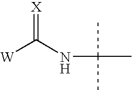
cannot be

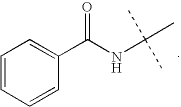

9. A compound chosen from 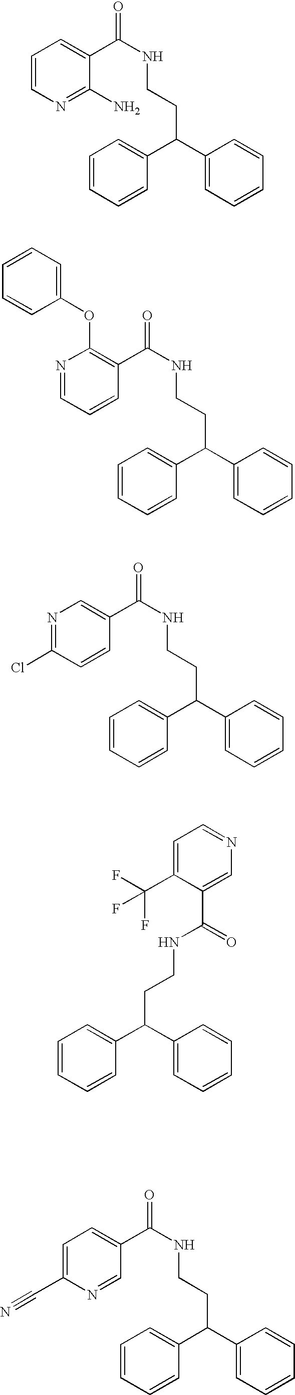 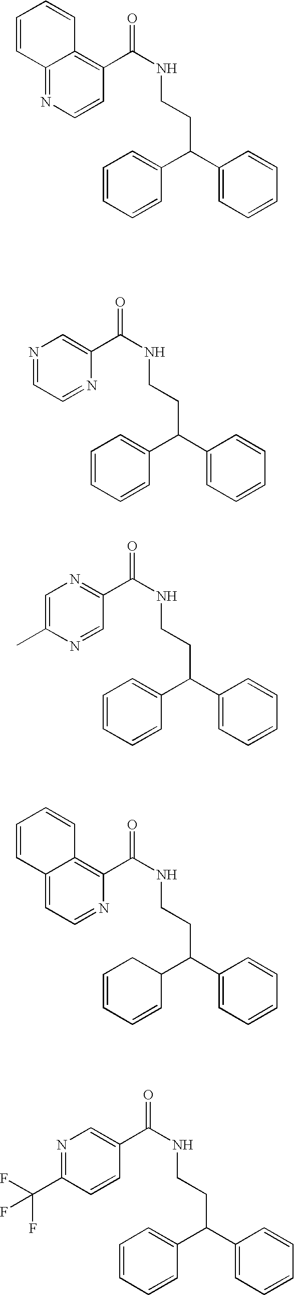 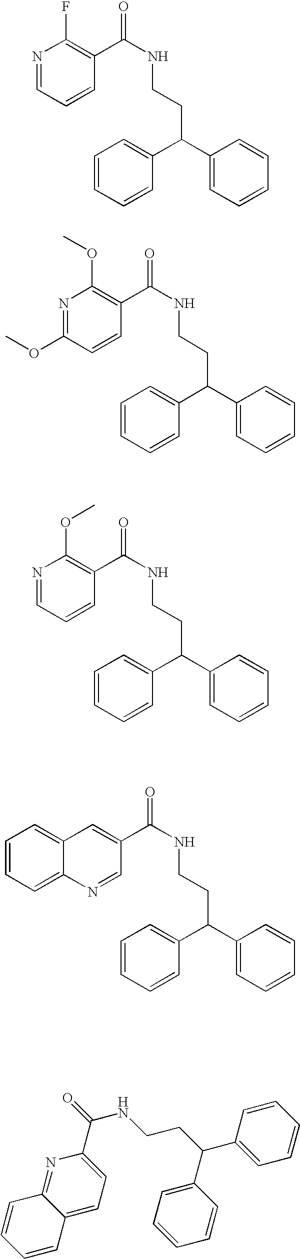 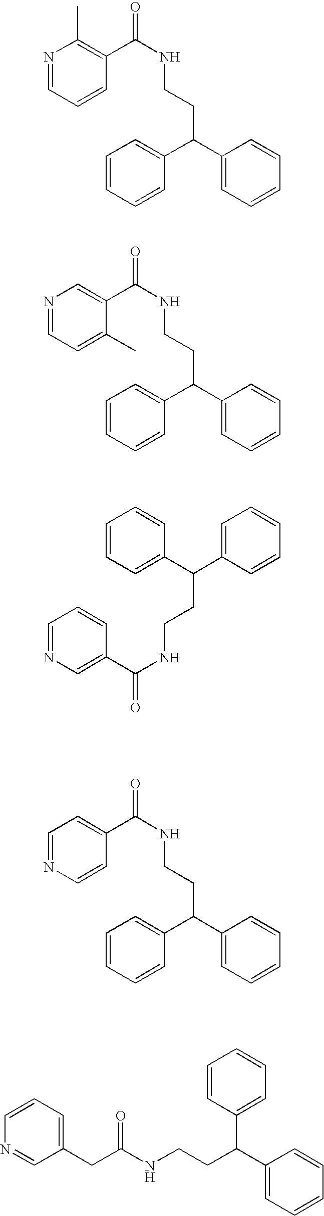 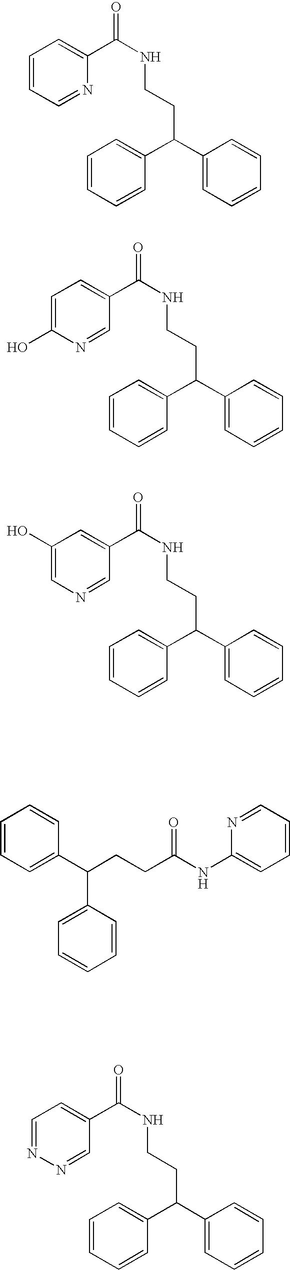 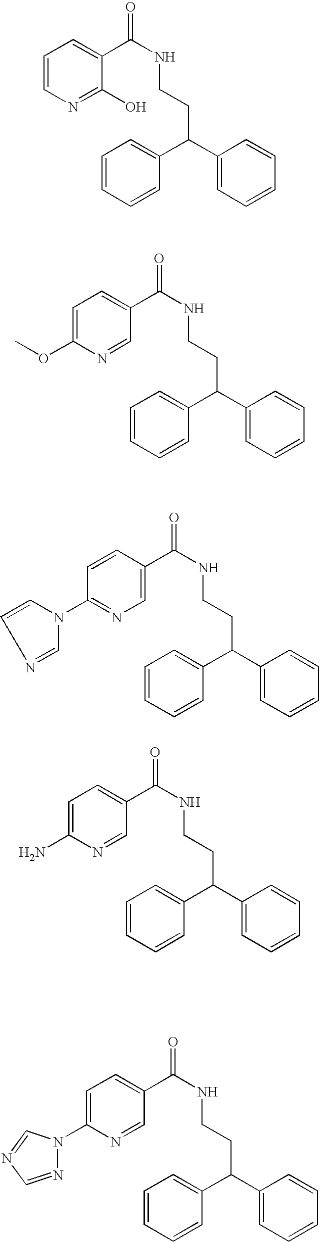 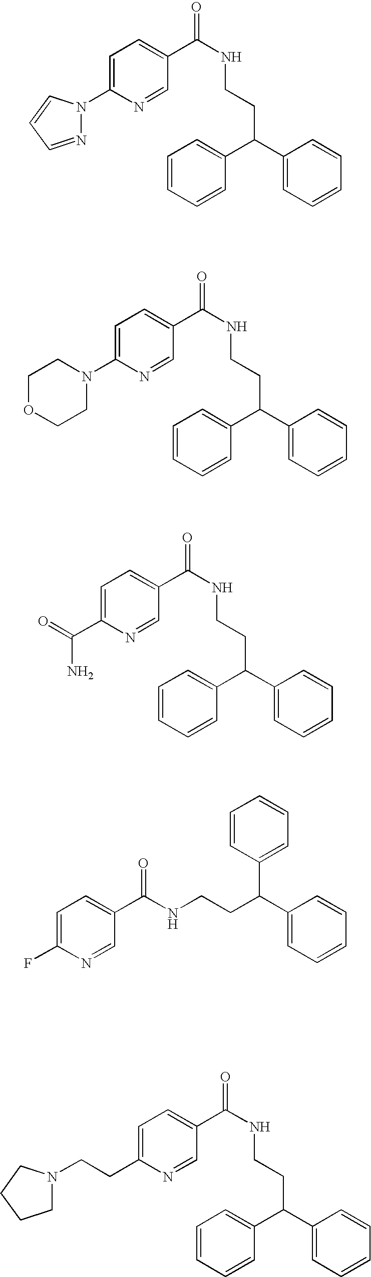 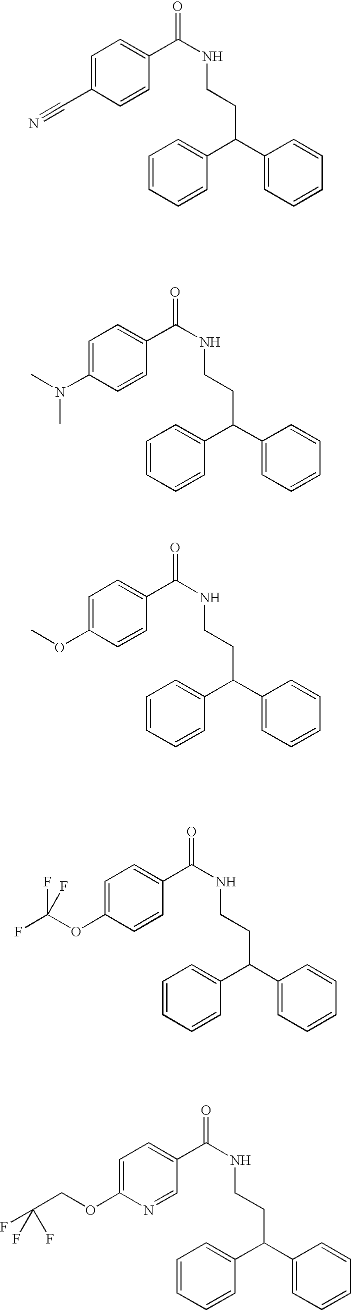 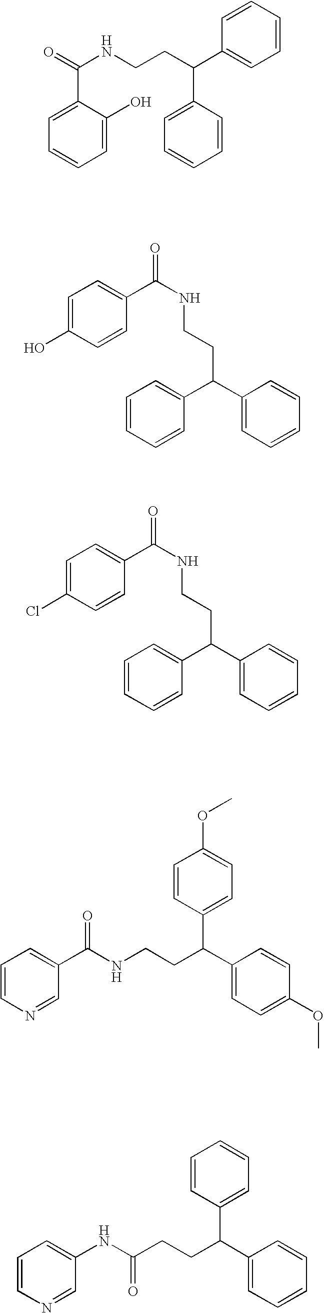 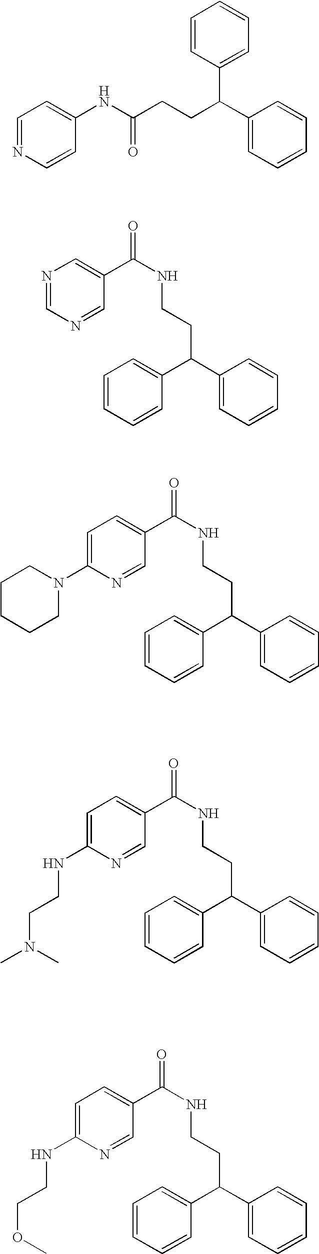 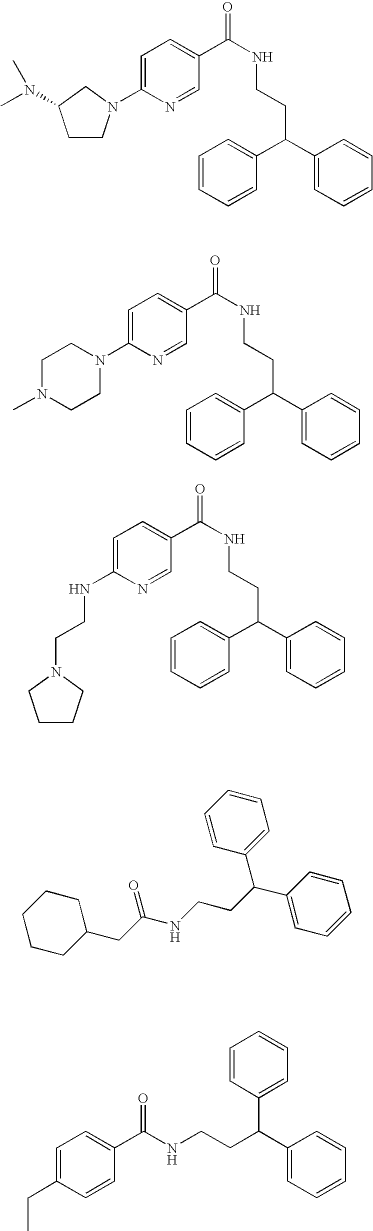 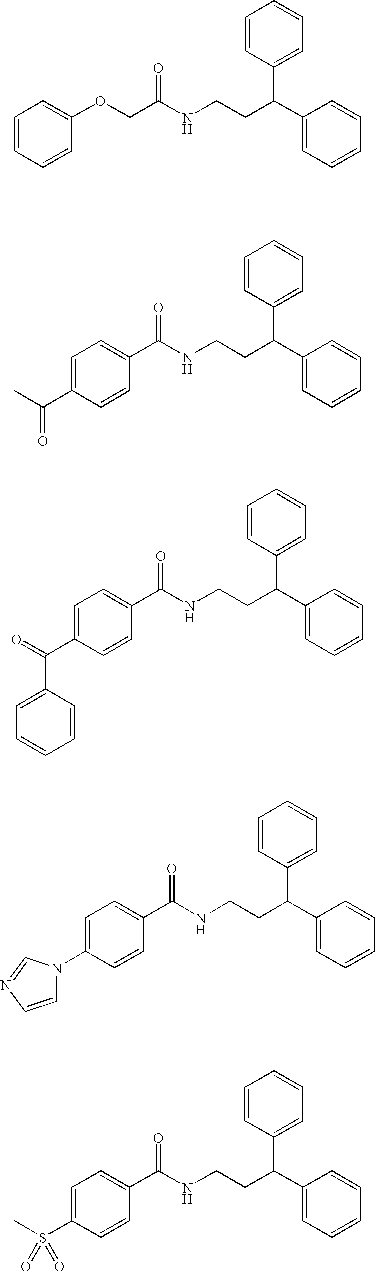 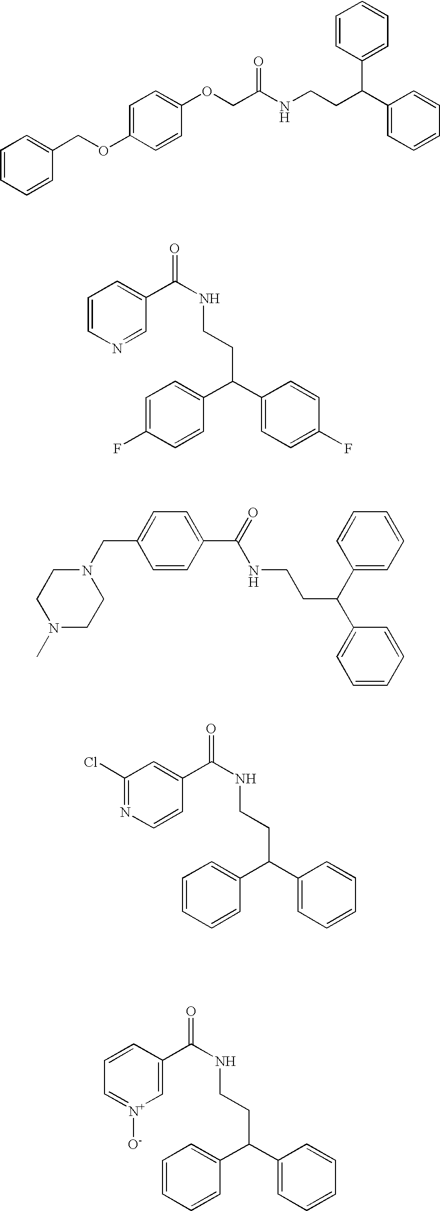 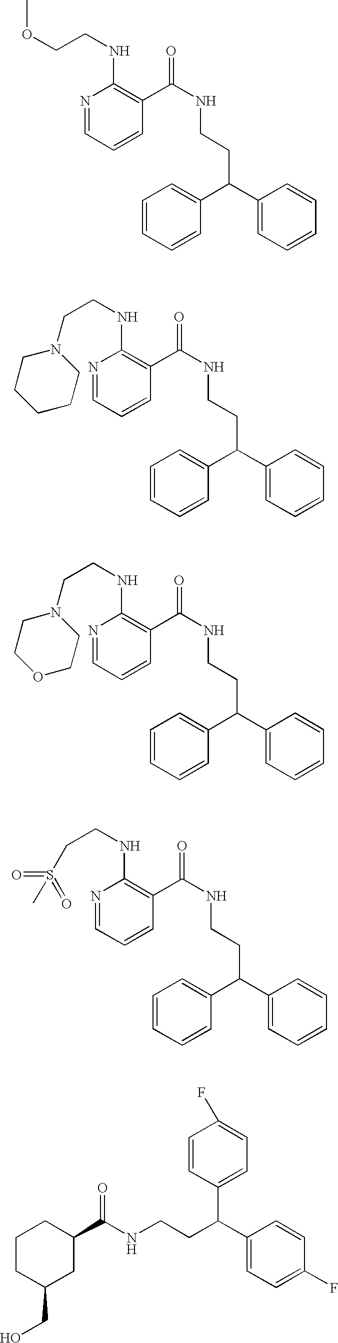 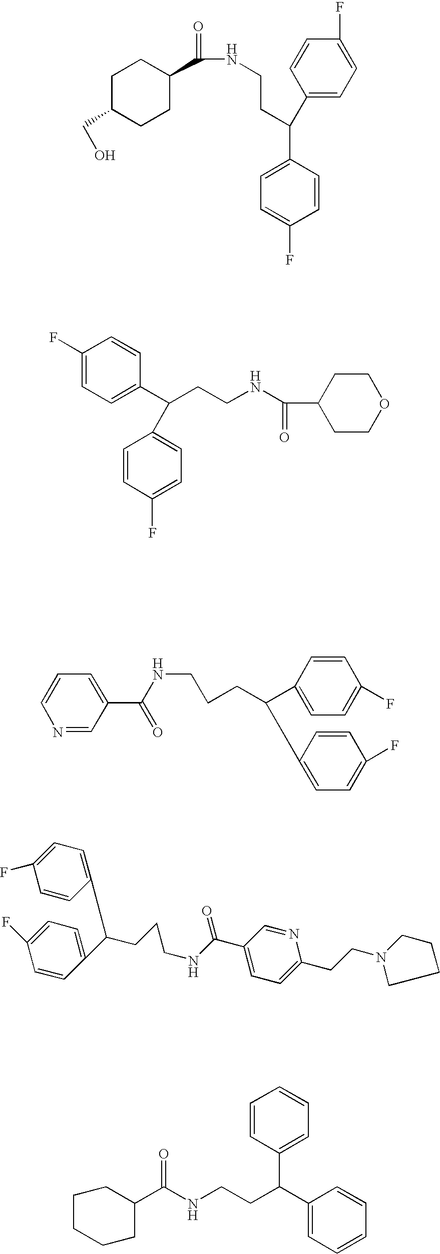 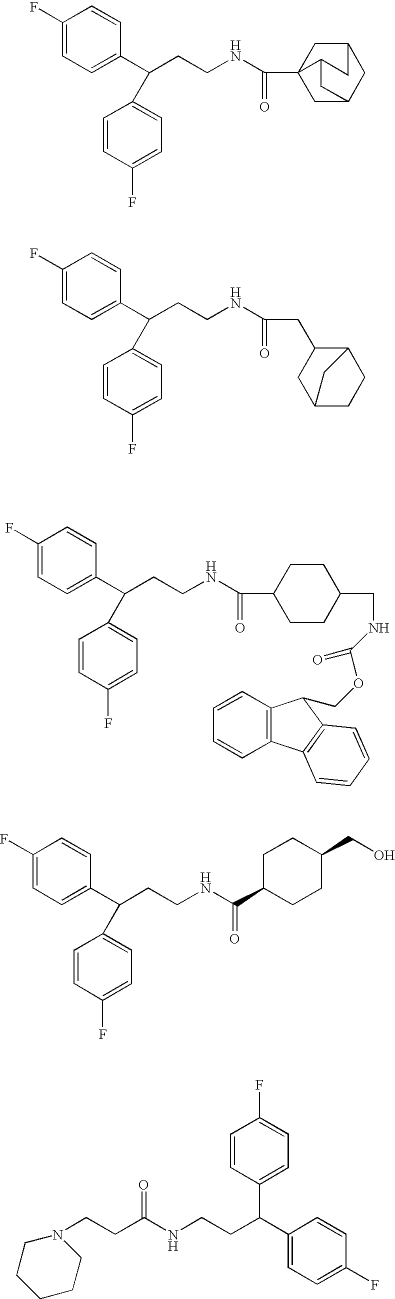 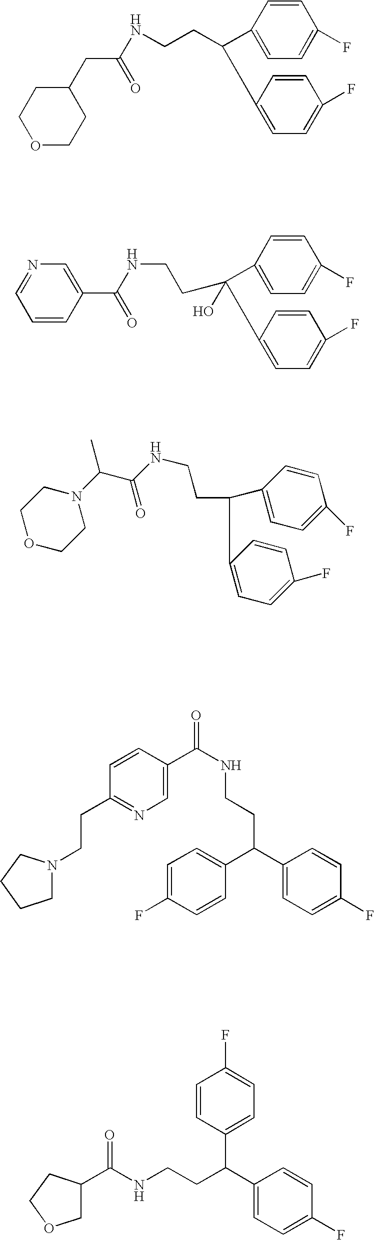 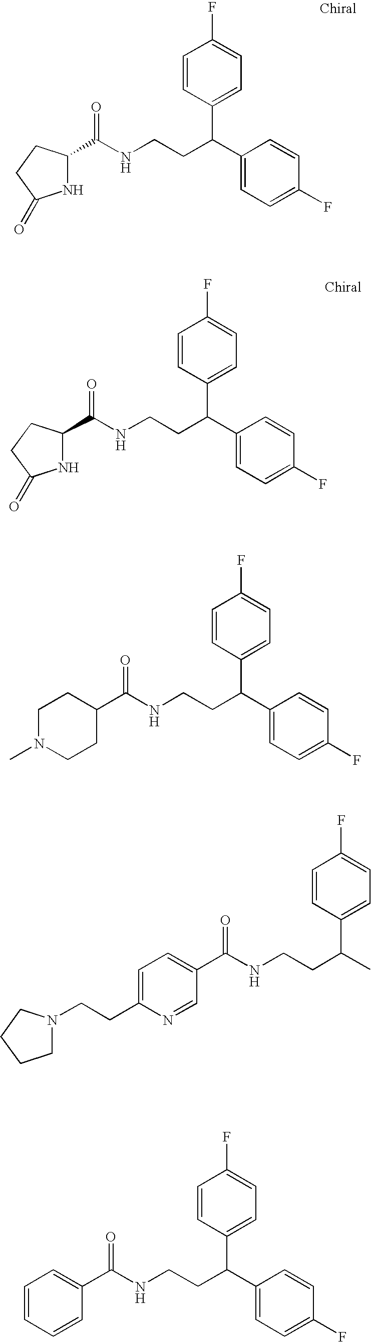 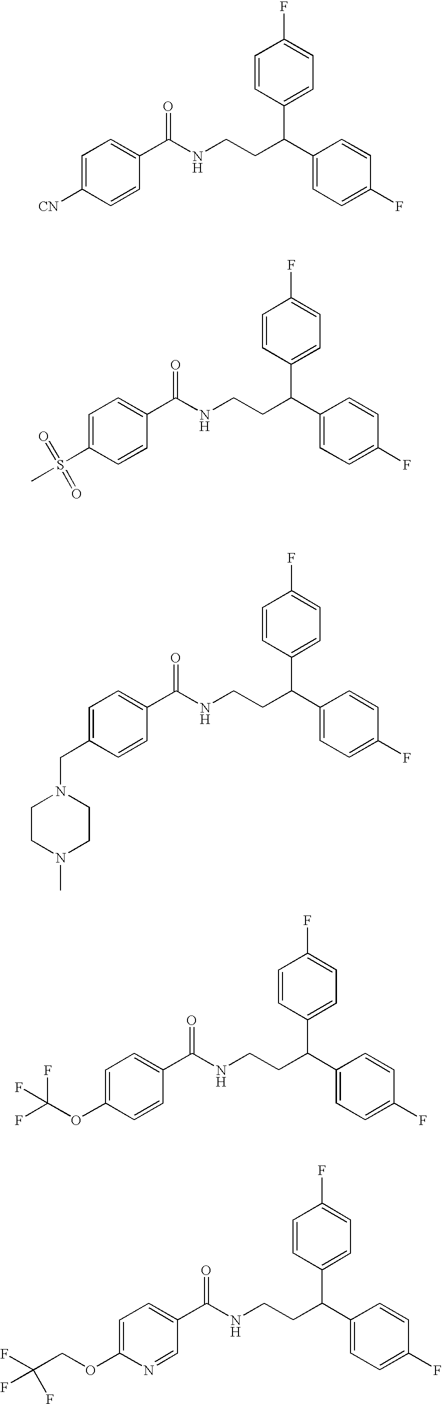 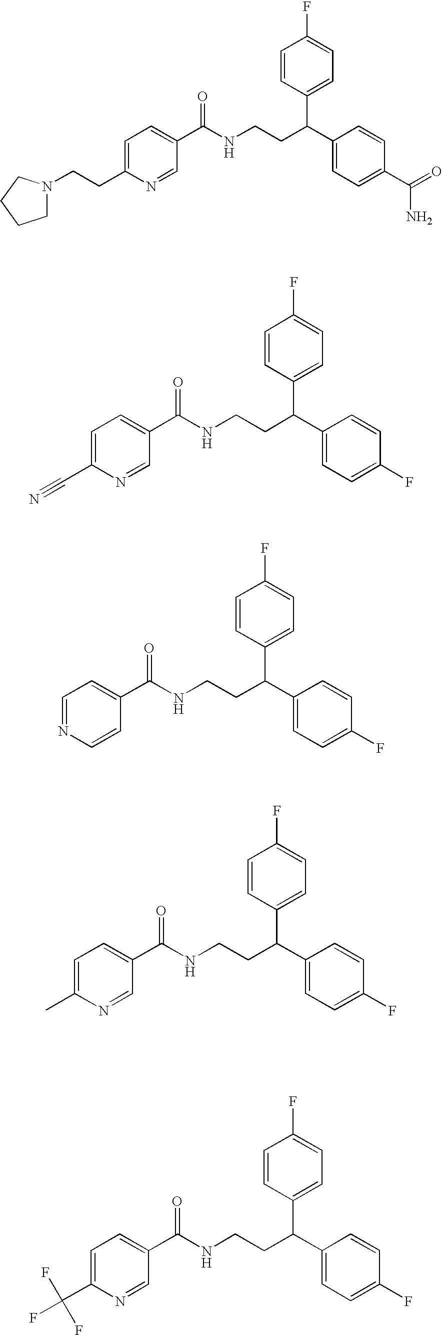 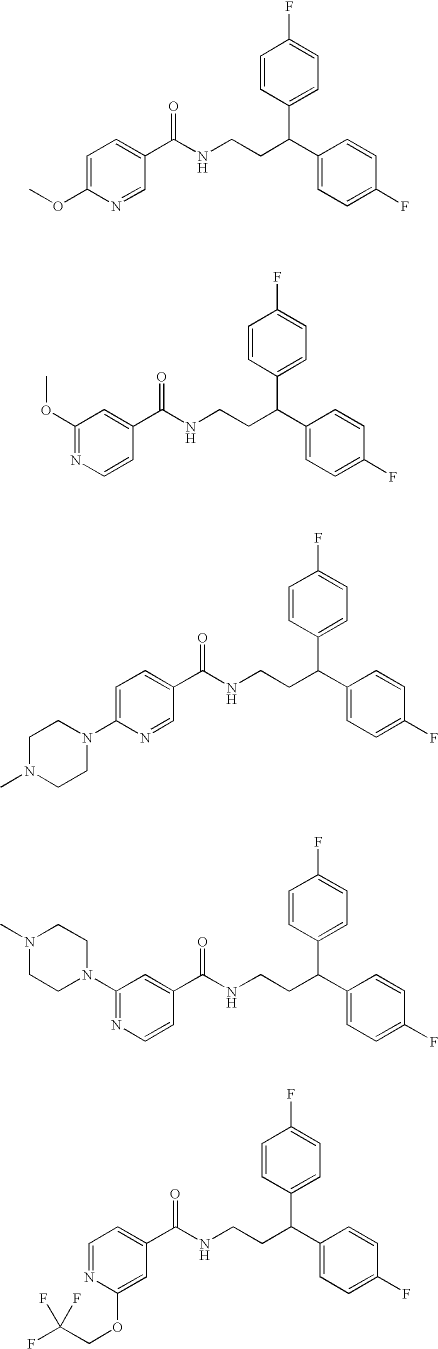 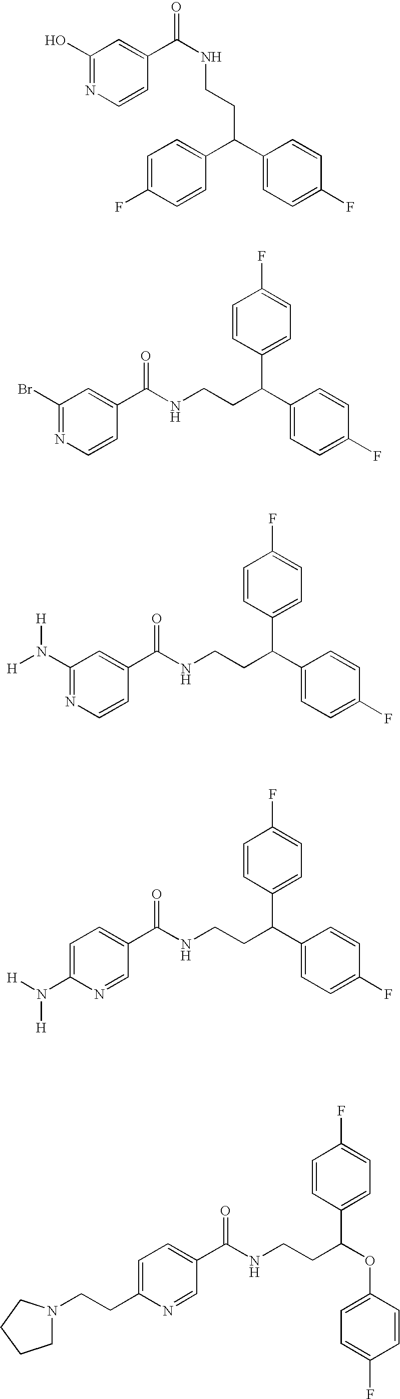 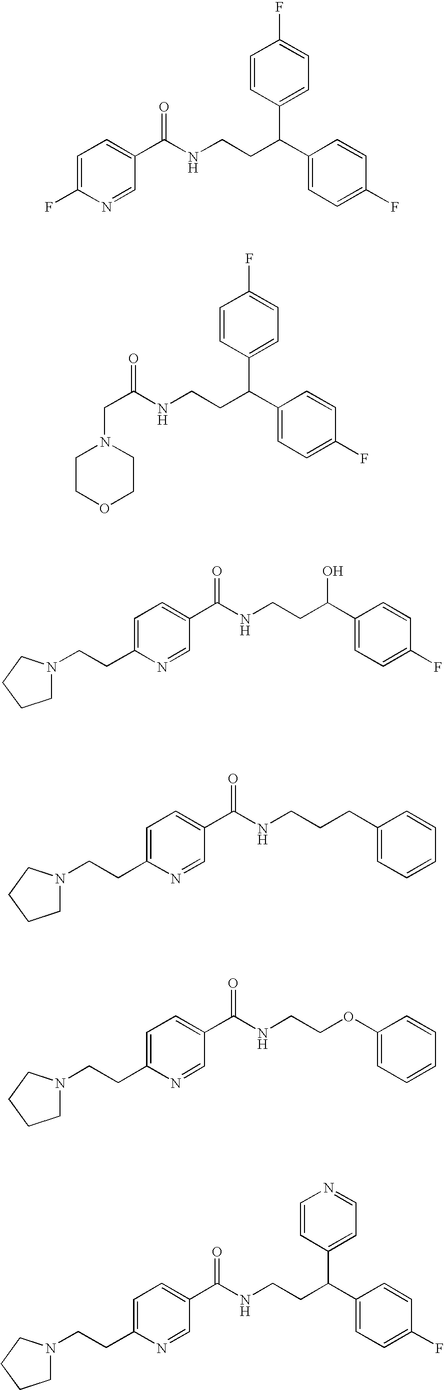 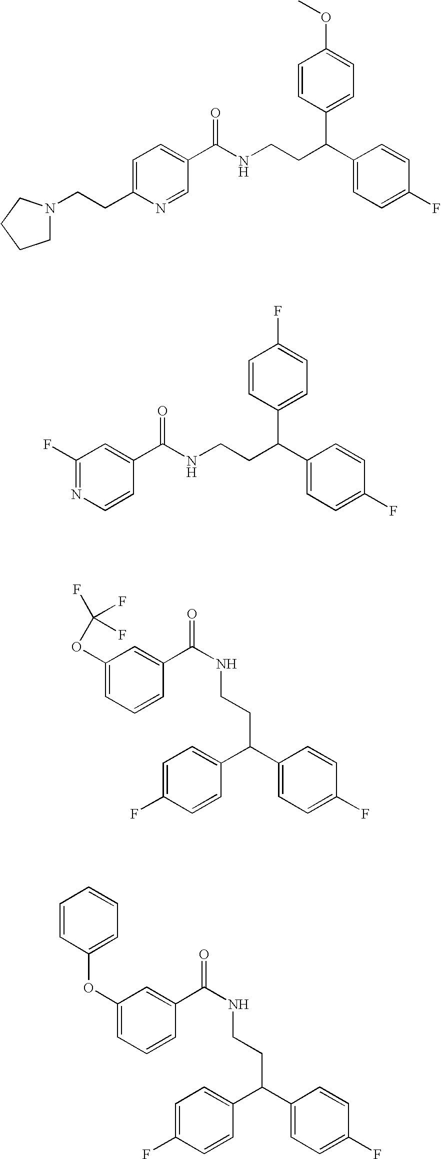 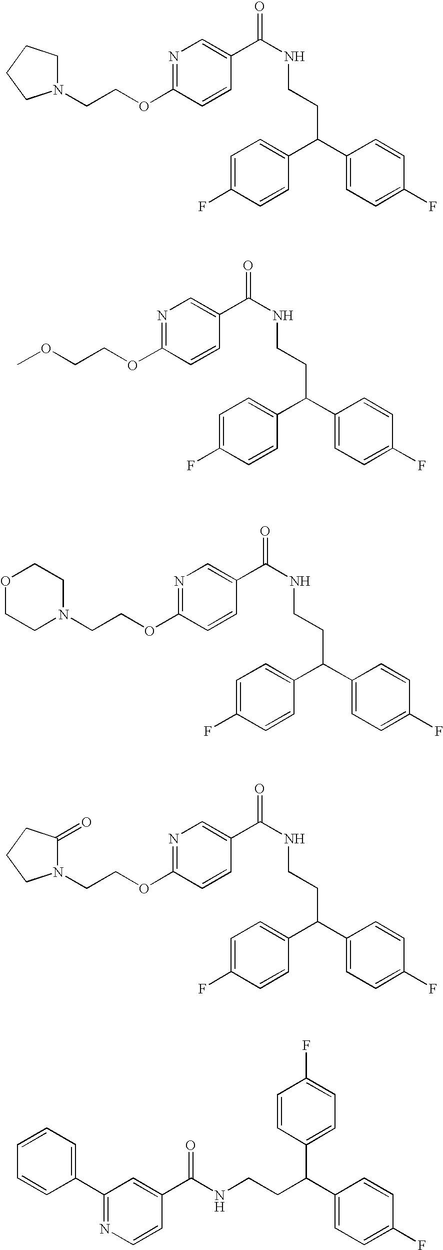 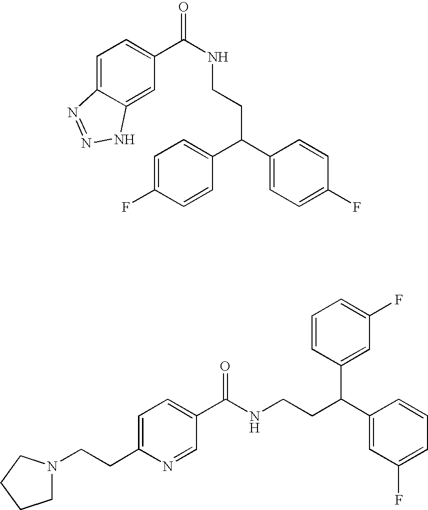
or the pharmaceutically acceptable salts thereof.

10. A method of treating a disease or condition chosen from type 1 and type 2 diabetes, insulin resistance syndrome, hypertension, atherosclerosis, coronary artery disease, angina, ischemia, ischemic stroke, Raynaud's disease and renal disease, said method comprising administering to a patient a pharmaceutically effective amount of a compound according to claim 7, 8 or 9.

11. A pharmaceutical composition comprising a pharmaceutically effective amount of a compound according to claim 7, 8 or 9 and one or more pharmaceutically acceptable carriers.
